(19) United States
(12) Patent Application Publication
(10) Pub. No.: US 2014/0030792 A1
(43) Pub. Date: Jan. 30, 2014

(54) Therapeutic Anti-Virus VLPS

(71) Applicant: Radhakrishnan Rathnachalam, Carmel, IN (US)

(72) Inventor: Radhakrishnan Rathnachalam, Carmel, IN (US)

(21) Appl. No.: 13/940,122

(22) Filed: Jul. 11, 2013

Related U.S. Application Data
(60) Provisional application No. 61/674,439, filed on Jul. 23, 2012.

(51) Int. Cl. C12N 7/00

(52) U.S. Cl. CPC C12N 7/00
USPC 435/236; 435/235.1

(57) ABSTRACT

This invention provides therapeutic viruses (TV) and methods to inhibit the propagation of a target virus. TVs can be rendered noninfectious by inactivating mutations, but also include sequences providing miRNA to inactivate essential mRNAs of the target virus. Methods can include provision of the TV and contact with a host cell harboring the target virus. The target virus providing the essential enzymes necessary to the replication of the TV and the TV disabling the target virus with the miRNA.

### CROSS-REFERENCE TO RELATED APPLICATIONS

This application claims priority to, and benefit of, U.S. provisional patent application U.S. Ser. No. 61/674,439, by Radhakrishnan Rathnachalam, filed Jul. 23, 2012.

### FIELD OF THE INVENTION

The present inventions are in the field of methods and constructs useful in targeting viruses with disabling virus like particles. The particles are directed to include disabled essential enzymes so they can only replicate in the presence of rescuing enzymes of a target virus. The particles can include sequences encoding an miRNA against a highly conserved sequence in an essential gene, and include a copy of the essential gene modified to avoid binding by the miRNA. The particles can be used in methods to inhibit an infections target virus from replicating.

### BACKGROUND OF THE INVENTION

The wikipedia (the free Encyclopedia http://en.wikipedia.org/wiki/) gives a brief description of “Discovery and development of HIV-protease inhibitors”. Presently, there are several treatment options for the HIV, such as nucleoside/nucleotide-based reverse transcriptase inhibitors (Freeman et al., (2004)), non-nucleoside reverse transcriptase inhibitors (Hopkins et al., (2004)), protease inhibitors (Craig, et al, (1991), Kempf et al, (1995)), fusion inhibitors (Wyatt, et al, (1994)), integrase inhibitors, maturation inhibitors, uncoating inhibitors, transcription inhibitors, translation inhibitors, and also combination therapies. These options include mainly small molecular enzyme inhibitors. They may require continuous intake of the medication for as long as the infection stays. Entry or fusion inhibitors disrupt the fusion of the virus and the target cell.

The compound enfuvirtide (Lalezari, et al. (2003)), a polypeptide and not a small molecule, has to be administered by injection. There are efforts using gene therapy targeting knock out of the CCR5 (Hütter, et al, (2009)) receptor. A curative approach will likely involve removing and treating stem cells with knockout genes for CCR5 and possibly CXCR4, administering high-dose chemotherapy to wipe out the existing HIV-susceptible immune system, and finally transplanting the modified stem cells to rebuild an immune system that is resistant to the virus. It has been observed that the HIV replication persists from anatomical sites where drug penetration is limited.

Antisense oligonucleotides were first used by Stephenson and Zamecnik (1978) in cell cultures, to inhibit RSV replication. Antisense mediated gene suppression against HIV-1 envelope gene has been reported in the literature Lu, et al, (2004). RNA interference (RNAi) provides post-transcriptional gene silencing (PTGS) and has been well described. It has been shown (Naito, et al, (2004), Zeng, et al, (2005)) that short (less than ˜22-26-mer) double stranded RNAs (dsRNA) are degraded without invoking an interferon response. RNAi binds to a protein to form a complex (RISC/RNA-induced silencing complex), which binds to mRNA transcripts with complementary nucleotide sequences (antisense) to the RNAi and degrades before the mRNA gets translated. Thus, RNAi-mediated post-transcriptional silencing offers a potentially powerful tool to inhibit viral replication. Guo and Kemphues (1995) and others (Zamore (2001), Novina, et al, (2002), Tuschl (2002), Zeng and Cullen (2002), Hemann, et al, (2003), Das, et al, (2004), Ge, et al, (2010), Saayman, et al, (2010), Liu, et al, (2011)) have described attempts to silence viral genes using small interfering RNAs (siRNA). Similar to the function of the synthetic siRNAs many groups (Lagos-Quintana, et al, (2001), Lau, et al, (2001), Zeng and Cullen (2002), Zeng, et al, (2002), Zeng and Cullen (2003), Lim et al, (2003), Zeng, et al, (2003), Zeng and Cullen (2004), Lim, et al, (2005), Creighton, et al, (2010)) have identified and worked with naturally occurring micro RNA (miRNAs), which have a different pathway compared to siRNA (Thompson (2002), Schwarz, et al, (2003)).

Many have identified and worked with small hairpin RNA (shRNA) (Paddison (2002), Boden, et al, (2004), Li, et al, (2007), Liu, et al, (2011)) to induce sequence-specific silencing in mammalian cells. The shRNA has basically a stem(up)-loop-stem(down) construct. The stem(up) and stem(down) are primarily reverse complementary to each other and will be paired. The antisense could be either the stem(up) or the stem(down) sequence. The difference between siRNAs and miRNAs is in their structures, where the siRNAs are double stranded, synthetic molecules needing perfect complementarity to function and miRNAs are single stranded, natural molecules that function even against partially complementary sequences. As a further development, the shRNAs have been reported to be part of a larger precursor sequence called pre-miRNAs from where the miRNAs are released (Lee, et al, (2003), Boden, et al, (2004), and Zeng et al, (2005²)).

There are two distinct steps involved in the culmination of the mature miRNA from the pre-miRNA. First the enzyme “drosha” cuts the paired “stem” and loop of the stem(up)-loop-stem(down) from the pre-miRNA. Approximately 22 nts are cut from the start of the loop. Then, the enzyme “dicer” cuts the loop and liberate the mature RNA. Also, “drosha” cuts the RNA sequence to create antisense sequences with an efficiency that depends on the length of flanking sequences on either side of the shRNA; the length of the sequences are to be greater than ˜60 nucleotide sequences (Lee, et al, (2003), Zeng, et al, (2005^{1,2}), Zeng and Cullen (2005), Feng, et al, (2011), Boden, et al, (2004)). Either the stem(up) or stem(down) or both could be mature RNAs. The stem(up) mature RNA has been designated as shorter stem hairpin RNA (L-sshRNA) (Khvorova, et al, (2003), Ge, et al, (2010)) where the antisense is placed at the 5′ end of the loop. The antisense placed at the 5′ side of the loop tend to be more potent, than those placed at the 3′ end of the loop.

In the quest to understand RNA and the protein partially involved in packaging, Aldovini and Young (1990) found that virus particles packaged with mutant sequences were unable to productively infect the cells even though the protein contents were similar to that of the infectious virus. There are many articles describing the achievement of making virus like particles (VLP) (Karacostas, et al, (1989), Haffar, et al, (1990), Carroll, et al, (1994), Haddrick, et al, (1996), Haselhorst, et al, (1998), Beckett and Miller (2007), Pal, et al, (2007), V Peremyslov and V Dolja (2007), Cornetta, et al (2008)). Georgens, et al, (2005) have proposed to use VLP as drug delivery systems by the inserting single chain Fv's or immunoglobulin binding domains or by covalently linking the active agents to VP2.

In light of these advances, there remain problems in treatment of viral diseases. For example, small molecule drugs are prone to resistance and may fail to penetrate all sanctuaries. Live virus vaccines may become pathogenic or cause mutations in normal cells as they incorporate into a genome. It would be desirable to have a focused and self limiting construct that destroys a virus's ability to replicate and spread, without the dangers of control loss or hazards to normal cells. The present invention provides these and other features that will be apparent upon review of the following.

### SUMMARY OF THE INVENTION

The present inventions include therapeutic viruses (TVs) and methods of their use in inhibiting infectious viruses in their host cells. The TVs can be incapable of infection due to inactivating mutations in essential genes, but rendered viable in the presence of the target infectious virus. The TVs can further encode a pre-miRNA, e.g., capable of antisense targeting of one or more essential target virus gene, e.g., while harboring a modified form of the gene not subject to inhibition by the miRNA. The methods include provision of the TVs and contacting them with cells infected by the target virus.

A TV capable of inhibiting propagation of a target virus can include an inactive essential gene for propagation of the TV in the host of the target virus (or have the gene essential for the propagation of TV absent or deleted), so that the TV can not propagate alone in the normal host of the target virus, but the TV can propagate in the presence the target virus providing of an active form of the essential gene. The TV can also include a sequence encoding a pre-miRNA, wherein an miRNA product from the pre-miRNA is has a first affinity for a highly conserved sequence of a gene in the infectious target virus, e.g., so that the miRNA inhibits translation of the highly conserved sequence. Further, the TV can have a modified version of the highly conserved target sequence, configured to transcribe an mRNA with a second affinity lower that the first affinity for the miRNA sequence, and which modified version of the highly conserved target sequence encodes an active form of the target sequence peptide product. Such a TV will fail to propagate in host cells without the presence of the target virus, yet also inhibit propagation of the target virus in the host cell when the TV is present. In this way, an infected cell will accept and replicate the TV, but after replication, the TV will prevent propagation of the infections virus.

In many embodiments, the TV is a modified version of the infectious target virus. For example, the TV can reflect the target virus, but be modified to inactivate a first essential gene product essential for propagation, and be modified at a second essential gene to increase mismatches to the miRNA, while retaining function of the second essential gene translation product peptide. The wherein the first essential gene and second essential gene can optionally be the same gene or other than the same gene. In many cases, the first essential gene is a gene associated with replication of the virus, e.g., a reverse transcriptase (RT), integrase, and/or an RNA dependent RNA polymerase (RDRP). In an embodiment, the target host cell encodes a functional reverse transcriptase, RNA dependent RNA polymerase (RDRP), and/or integrase enzyme, and the TV does not. Optionally the second essential gene can include genes associated with replication of the virus and/or structural genes.

In certain preferred embodiments, the TV is deficient in at least two enzymes necessary for replication in a host cell for the target virus. In this way, a mutation reactivating one enzyme gene will fail to rescue independent viability of the TV.

In many embodiments, the pre-miRNA is adapted to provide the miRNA product by the presence of Dicer or Drosha cutting sites in the pre-miRNA.

In certain embodiments, the miRNA, or pre-miRNA has at least 85% identity to UAUUGCUGGUGAUCCUUUCCA (SEQ ID NO: 1), CUGUCCAUUUAUCAGGAUGGAG (SEQ ID NO: 2), CCAAUCCCCCCUUUUCUUUUAAA (SEQ ID NO: 3), AUACUGCCAUUUGUACUGCUGU (SEQ ID NO: 4), and/or a complementary sequence thereof.

In certain particular embodiments for TVs against human immunodeficiency virus (HIV) the TV can have at least 85% identity to the sequence of FIG. 30, while retaining at least 95% identity to sequences (e.g., underlined sequences) we have identified as useful in, e.g., rendering the TV non-infectious and/or avoiding antisense inhibition of the miRNA. These sequences can be configured to retain protein structures and provide appropriate nucleic acid filling of capsid during virus processing. In this way, TV can be adapted to inhibit replication of HIV when both the TV and HIV are present in the same cell, and to provide production and release of replicate TV to inhibit HIV in additional infected host cells.

In another particular embodiment, the TV has at least 85% identity to the sequence of FIG. 36 or of FIG. 37, while retaining at least 95% identity to sequences (e.g., capitalized sequences) we have identified as useful in, e.g., rendering the TV non-infectious and/or avoiding antisense inhibition of the miRNA. These sequences can be configured to retain protein structures and provide appropriate nucleic acid filling of capsid during virus processing. In this way, TV can be adapted to inhibit replication of hepatitis type C (HCV) when both the TV and HCV are present in the same cell, and to provide production and release of replicate TV to inhibit HCV in additional infected host cells.

Also included in the present invention are methods of inhibiting propagation of infectious viruses. A method of inhibiting replication of a target infectious virus can include providing a TV of the invention and contacting the TV with a cell infected with the target infectious virus. For example, the method can include providing a therapeutic virus (TV) comprising an inactive essential gene for propagation in the host of the target virus (or having an inactivated or missing version of essential gene) in the normal host of the target virus. In this way, the TV can not replicate alone in the normal host of the target virus but the TV can propagate in the presence the target virus, which provides a copy of an active form of the essential gene. The TV further includes a sequence encoding a pre-miRNA, e.g., cleavable to provide an miRNA product with an antisense affinity for a highly conserved sequence of the target virus in a gene of interest (typically an gene essential for replication or propagation of the target virus) so that the miRNA can inhibit translation of the highly conserved sequence. In addition, the TV can include a modified version of the highly conserved target sequence adapted to transcribe into a mRNA with less affinity (e.g., antisense binding) for the miRNA sequence. This feature can provide for continued TV propagation with an active form of the target sequence peptide product while the target virus is previously disabled by the TV. On contact with the provided TV, a target virus infected cell can be inhibited from taking part in further progression of the target virus infectious progression.

In certain aspects of the methods, the TV is adapted to be deficient in at least two enzymes necessary for replication in a host cell for the target virus. The TV can be a modified version of the target virus, e.g., with an inactivated or deleted first essential gene product, and a second essential gene modified to increase antisense mismatches to the miRNA while retaining function of the second essential gene translation product peptide. In many cases, the first essential gene and second essential gene are other than the same gene. The first essential gene and second essential gene can be the same gene or different genes. For example, in the methods, first essential genes are directed to initial gene replication and/or integration functions, e.g., reverse transcriptases (RT), an integrases, and/or an RNA dependent RNA polymerases (RDRP).

In the methods, the pre-miRNA can be converted into the miRNA by cutting with Cutter or Drosha.

The methods can include inhibiting translation of the highly conserved sequence by hybridization of the miRNA to an mRNA of the target virus, which mRNA encodes the highly conserved sequence. The methods can further comprise modifying the highly conserved target sequence to provide the modified version in the TV by changing codon triplet codes to alternate codons encoding the same amino acid. The methods can further comprise adapting the miRNA to not bind to the modified TV highly conserved target sequence under intracellular host cell conditions.

The present inventions include identification of highly conserved stretches of nucleotides using the multiple sequence alignments to select sites for creating antisense sequences by calculating the highest percentage nucleotide frequency at each location of the entire length of the genome of interest. A moving window of desired length is used to find the maximum scoring stretches.

In designing shRNA to produce the designed (antisense) miRNA, the orientation of the miRNA can include, e.g., stem(up), at least 3 nucleotides (nts) at the 3′ end of the designed miRNA, the loop sequences, and at least 3 sequences following the loop sequence are identical from with the corresponding site in the designed shRNA. In designing shRNA to produce the designed (antisense) miRNA, the orientation of the miRNA can be, e.g., stem(down), at least 3 nts at the 5′ end of the designed miRNA, the loop sequences, and at least 3 sequences before the 5′ end of the loop sequence are identical from with the corresponding sites from an observed shRNA including the ones in a public database. For example, see FIG. 1.

In designing pre-miRNAs, the flanking sequences can be, e.g., of length at least 6 nts adjoining 5′ end of the miRNA of the designed pre-miRNA, the orientation of the designed miRNA being stem(up), at least 3 nucleotides at the 5′ end of the designed miRNA, at least 3 nucleotide sequence opposing this 5′ end sequences, and at least 6 nts following the opposing sequence are identical to the corresponding sites from an observed pre-miRNA including a pre-miRNA, e.g., from a public database.

In designing pre-miRNAs, the flanking sequences can be, e.g., of length at least 6 nts adjoining 3′ end of the miRNA of the designed pre-miRNA, the orientation of the designed miRNA being stem (down), at least 3 nucleotides at the 3′ end of the designed miRNA, at least 3 nucleotide sequence opposing this 3′ end sequences, and at least 6 nts followed by the opposing sequence are identical to the corresponding sites from an observed pre-miRNA including a pre-miRNA, e.g., from a public database.

The present inventions include a pharmaceutical composition comprising, or containing within it, a therapeutically effective amount of a product described above.

The inventive method can include design for a mutant non-replicative virus or pathogen to deliver a therapeutic composition comprising of at least one product of claims 2-8 to infected cells. The catalytic site or substrate binding sites of at least one of the enzymes either maintained or coded for by the therapeutic virus or pathogen, are sufficiently modified with mutations, insertions, or deletions either by site specific changes or by natural selection, so that the enzyme is inactive and the chances of regaining activity is very low.

Another aspect of this invention is to protect the therapeutic non-replicative virus or pathogen. The nucleotides corresponding to the selected conserved sites identified above can be modified by insertions, deletions, and/or mutations so that there is a high dissimilarity at nucleic acid level and high similarity at the amino acid level between the modified site of the therapeutic virus or pathogen and the antisense miRNA designed for that site.

Another aspect of this invention is to completely or partly remove the nucleotides corresponding to the gene of the enzyme from the therapeutic non-replicative virus or pathogen that the antisense miRNAs were developed against.

One or more of the miRNA or shRNA, or pre-miRNA of described above can be introduced into the untranslated region of the virus' or pathogen's genome.

The inventions contemplate using a modified form of a noninfectious virus or pathogen, such as in a combination of embodiments described above, to deliver the pre-miRNAs and in turn miRNA to a host cells.

The modified forms of a virus or pathogen can be allowed to produce the designed miRNAs and propagate the designed miRNA automatically from within the cell either by integration into the host genome or by forming a separate plasmid.

Products above can optionally be in the form of RNA or DNA, as appropriate, e.g., to the particular infectious virus target.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a surface” includes a combination of two or more surfaces; reference to “bacteria” includes mixtures of bacteria, and the like.

Keywords used in this description of the inventions, and well known to those skilled in the art include: antisense, dicer, Drosha, dsRNA, mirbase, miRNA, MSA, pre-miRNA, PTGS, RNAi, shRNA, TV, UTR, and VLP.

### ABBREVIATIONS

CCR5—chemokine receptor 5
CXCR4—CXC chemokine receptor 4
dsRNA—double stranded RNA
HIV-1—human immuodeficiency virus type-1
miRNA—micro RNA

### MSA—Multiple Sequence Alignment

PTGS—post-transcriptional gene silencing
RISC—RNA-induced silencing complex
RNAi—RNA interference

### RSV—Rous Sarcoma Virus

### RT—Reverse Transcriptase

shRNA—short hairpin RNA
sshRNA—shorter stem hairpin RNA

### TV—Therapeutic Virus

UTR—Untranslated region

### VLP—Virus Like Particles

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill), or by visual inspection.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores for nucleotide sequences are calculated using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff (1989) Proc. Natl. Acad. Sci. USA 89:10915).

In addition to calculating percent sequence identity, the BLAST algorithm can also perform a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. Nucleic acids are considered similar to, and within the purview of the present invention, if they are similar to unique nucleic acids of the invention with smallest sum probability of than about 0.1, preferably less than about 0.01, and more preferably less than about 0.001.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

### BRIEF DESCRIPTION OF THE DRAWINGS

FIG. 1 is a schematic representation of a model pre-miRNA containing shRNA and miRNAs.

FIG. 2 is a schematic representation of an HIV genome.

FIG. 3 is a schematic representation of a modified HIV genome designed to act as a therapeutic virus against HIV.

FIG. 4 provides the genomic sequence for HIV-1 (LOCUS HIVU69584) (gb: U69584.1).

FIG. 5 provides a model of the active site of RT in stereo with mutated residues to inactivate the enzyme based on 2zd1.pdb.

FIG. 6 provides a model of the active site of RT in stereo with mutated residues to inactivate the enzyme based on structure 1rtd.pdb.

FIG. 7 presents a suggested maximally conserved first pre-miRNA segment selected in reverse transcriptase (2zd1.pdb).

FIG. 8 shows the predicted secondary structures for: (a) the hsa-mir-4309 which has a matching sequence at the 3′ end to the designed antisense (TATTGCTGGT GATCCTTTCCA (SEQ ID NO: 5)) for the first conserved segment in RT; (b) hsa-miR-32 having matching nucleotides at the 5′ end of our antisense; and, (c) the shRNA of the designed antisense. The black dots are the dicer cleaving site, and the stars indicate the drosha cleaving site. These sites are circled to show that they are maintained in the design to resemble the natural miRNA sites.

FIG. 9 shows the RT segment 1 pre-miRNA sequence with loop sequences from hsa-mir-4309 and flanking sequences derived from hsa-mir-32.

FIG. 10 shows a sequence for the hiv-1 Reverse Transcriptase (RT1) of the TV. Mutations to inactivate are underlined. Mutations to prevent binding of antisense developed for the first conserved site are double underlined.

FIG. 11 shows a suggested maximally conserved second segment in reverse transcriptase, close to the active site. The substrate is shown in dots.

FIG. 12 shows suggested mutations to the second segment in RT to avoid self destruction by the designed antisense sequences.

FIG. 13 shows the predicted structure of the second segment of RT, loop based on hsa-mir-4660 and flanking sequences based on hsa-mir-2054. CTGTCCATTT ATCAGGATGGAG (SEQ ID NO: 6). (a) hsa-mir-4660 (for loop); (b) hsa-mir-2054 (for flanking); and, (c) designed miRNA/shRNA.

FIG. 14 shows a suggested second site for RT-miRNA hit with flanking sequences and the antisense sequence for the second segment of RT with its sequences.

FIG. 15 shows suggested sequences for the hiv-1 Reverse Transcriptase (RT2) of the TV. Mutations to inactivate are underlined. Mutations to prevent binding of antisense developed for the second conserved site are double underlined.

FIG. 16 shows a suggested sequence for the hiv-1 Reverse Transcriptase (RT3) of the TV. Mutations to inactivate are underlined. Mutations to prevent binding of antisense developed for both the first and the second conserved sites are double underlined.

FIG. 17 shows a structure diagram presenting the active site of HIV-1 integrase from 1qs4.pdb.

FIG. 18 shows a suggested first segment based on highly conserved Hiv-1 integrase sequences.

FIG. 19 shows conserved site for the first Segment of Integrase adjusted to get good matches for the 5′ and 3′ ends. (CCAATCCCCCCTTTTCTTTTAAA (SEQ ID NO: 7))

FIG. 20 presents the predicted secondary structures of: (a) hsa-mir-1245b-5p for the design of loop for the first segment of antisense (CCAATCCCCCCTTTTCT TTTAAA (SEQ ID NO: 8)) for the integrase; (b) hsa-mir-4275 to model the flanking sequences; and, (c) for the shRNA of the antisense for the first conserved segment for integrase. Black dots denote the scissile bonds for the dicer, and the dark stars the scissile bonds for drosha.

FIG. 21 presents steps involved in the design of a suggested pre-miRNA containing antisense for the first conserved segment for integrase.

FIG. 22 provides a suggested sequence for the hiv-1 Integrase (IN1) of a TV. Mutations to inactivate are underlined. Mutations to prevent binding of antisense developed for the first conserved site are double underlined.

FIG. 23 presents a suggested Hiv-1 integrase second segment in a relatively highly conserved region.

FIG. 24 presents a Hiv-1 integrase second segment in a relatively highly conserved as seen in the structure 3nf6.pdb.

FIG. 25 shows the predicted secondary structure of selected miRNA (a) hsa-mir-4705 for integrase segment 2 loop sequences; (b) hsa-mir-4693-5p for flanking sequences; and, (c) for the shRNA of the designed miRNA. The parts that are to be conserved for loop design and the flanking residues design are enclosed in dotted circles. The black filled circle is the scissile bond for drosha, and the black star is the scissile bond for the dicer. Antisense for second segment for integrase is ATACTGCCATTTGTACT GCTGT (SEQ ID NO: 9).

FIG. 26 presents a procedure to select a pre-miRNA sequence for the antisense of the integrase second site. Loop sequences were combined from hsa-mir-4705 and the flanking sequences from hsa-mir-4693.

FIG. 27 presents a suggested sequence for the hiv-1 Integrase (IN2) of the TV. Mutations to inactivate are underlined. Mutations to prevent binding of antisense developed for the first conserved site are double underlined.

FIG. 28 presents a consensus sequence for integrase (IN3) with modifications to inactivate the enzyme as well as modifications made to both the conserved sites to avoid self binding of the antisense sequences.

FIG. 29 shows constructs for suggested antisense sequences. Two sequences are provided for the reverse transcriptase and two for the integrase, for insertion into the genomic sequence for the therapeutic virus.

FIG. 30 presents a full sequence for a suggested version attenuated therapeutic virus against HIV. The Reverse Transcriptase starts at 2096; RT has a length shorter by 6 nts compared to the wild type. The Integrase starts at 3776 instead of 3770; the integrase domain ends at 4642. The Nef domain starts at 8378; residue R35 cga changed to A gcc; D36 gac to S tca; Residue D174 gac to R aga. Introduce the antisense sequences (not numbered) for RT1 RT2 IN1 IN2 at 8998. The 3′ end LTR starting at 9193 is underlined with double-waves.

FIG. 31 is a schematic diagram showing the organization of a Hepatitis C virus genome.

FIG. 32 is a schematic diagram presenting a suggested conserved Hep C site in ns5b rdrp (4dru.pdb). The inhibitor is shown in dark black stick model.

FIG. 33 shows a suggested conserved stretch for the ns5b an rdrp for modification in an anti-HepC TV.

FIG. 34 shows the predicted secondary structure for the first conserved stretch for NS5B, and the design of pre-miRNA for its antisense. (a) hsa-mir-376a; (b) hsa-mir-32; and, (c) designed shRNA for the antisense TATTGATTTCACCTGGAGAGTA (SEQ ID NO: 10).

FIG. 35 outlines steps involved in the design of the pre-miRNA for the conserved site within RDRP of HCV.

FIG. 36 presents a suggested final sequence for the therapeutic HCV virus. The pre-miRNA are in shown in capital letters starting at 7608.

FIG. 37 presents a suggested final sequence for the therapeutic HCV virus. The pre-miRNA are shown in capital letters starting at 9378.

### DETAILED DESCRIPTION

Per our design, the therapeutic virus (TV) has to be non-infectious to normal cells not infected by the pathogenic virus. This condition is achieved by selectively mutating, deleting, and/or inserting residues lining the active site of one or more of the enzymes essential for the propagation of the infectious virus. The TV should be able to permeate (access) all normal host cells as does the infectious virus to maintain selectivity and specificity. Therefore, any modifications to the enzymes of the virus should not impart significant changes to the three dimensional structure of the enzymes and in turn packing within the capsid. Because of this, the shape and characteristics of the surface of the TV should closely resemble that of the infectious virus. As the TV is missing enzymes essential for the propagation, e.g., no functional reverse transcriptase (RT) and/or integrase enzymes, it depends on existence of RT and integrase in that cell, possible only in those cells that have already been infected by a target virus, e.g., such as HIV-1. Once the essential enzymes are available in a cell, the genome of the TV can be propagated, e.g., by incorporation into the host cell's genome, in a fashion similar to that of the infectious target virus.

The TV can have pre-miRNAs inserted into its UTR region. In the case of a TV for HIV-1, the miRNA can be inserted after the stop codon of the Nef domain and become part of the host genome. When the TV genome is replicated, mRNAs would be produced for the pre-miRNA portions and processed by the dicer and drosha enzymes. The resulting miRNAs bind to the transcripts of the infectious virus and interfere with the production of those key viral enzymes leading to the propagation of the infectious virus.

The miRNA binding sites can also be modified in the TV, so that they would not interfere with the production of the TV. During this process, the TV should be packaged and secreted. The secreted TV would affect more cells to propagate their beneficial function. The production of the TV continues until the infected cells die. This aspect of this method would have long term benefits. On the other hand, the TV would not affect healthy cells. We believe that production of TV would stop after clearing all of the infectious virus and the depletion of functional RT and integrase in the host cells. Therefore, patients need not take the treatment continuously for a long term, rather than a few rounds of the therapy.

The next aspect of our design for, e.g., retrovirus therapy is that the miRNAs will not interfere with the production of TV. By design, we have selected to mutate only the active site residues to render RT and integrase inactive. The miRNAs, targeting a particular stretch of the gene of those enzymes from the infectious virus, would also bind to the genes of the enzymes from the TV and stop their production. To avoid this outcome, we have modified those binding sites within the gene of the enzymes of the TV not to bind the designed miRNAs.

Like any virus, the TV would undergo random mutations rendering the process ineffective after a few cell cycles. To address this issue, we have targeted more than one conserved site and created the corresponding miRNAs to reduce the potential for such inactivation to occur.

Another design aspect is the crossover between the infectious and therapeutic virus. Even if the TV acquires a copy of the gene of an active enzyme, it would be targeted by the miRNAs and destroyed.

Another aspect of the method is having an immune response just like what the infectious virus would face. Though, there will be an immune response, the total number of infective virus would start decreasing once the therapy starts working.

Another important issue is the insertion of the TV at wrong locations within the host genome and this could very well happen from the infectious virus within the patient and so there is no additional risk to what already exists within the infected cells.

### Expression of Virus Like Particles.

In the quest to understand RNA and the protein partially involved in packaging, Aldovini and Young (1990) found that virus particles packaged with mutant sequences were unable to productively infect the cells even though the protein contents were similar to that of the infectious virus. There are many articles describing the achievement of making virus like particles (VLP) (Karacostas, et al, (1989), Haffar, et al, (1990), Carroll, et al, (1994), Haddrick, et al, (1996), Haselhorst, et al, (1998), Beckett and Miller (2007), Pal, et al, (2007), V Peremyslov and V Dolja (2007), Cornetta, et al, (2008)). Georgens, et al, (2005) have proposed to use VLP as drug delivery systems by the inserting single chain Fv's or immunoglobulin binding domains or by covalently linking the active agents to VP2.

Steps involved in the method of designing of therapeutic virus (TV):

1) Inactivate the virus by inactivating one or more of its essential enzymes.
2) Identify the linear sequence within the gene coding for the enzyme to create an antisense miRNA that would bind to this sequence.
3) Modify that stretch within the genome of the TV to protect the TV from the designed antisense miRNA.
4) Design the pre-miRNA for the antisense miRNA.

### Inactivation of the Virus.

One of the features of the TV is that it should not be active in normal uninfected cells. To this end, we have decided to inactivate one or more of the enzymes that are essential for the activity of the infectious virus. Our strategy is to identify and mutate those residues that line the active site, so that the enzyme is rendered inactive, while not affecting the folding of the enzyme. Crystal structures of these enzymes, when available, provide information to design these inactive enzymes. One may also get an idea of possible mutations from analyzing the sequence alignments of the enzyme with highly homologous enzymes. Mutations to the active site could be designed through directed evolution. By employing one or more of these techniques, one should be able to modify the enzyme to have desired properties.

Identification of an miRNA Target.

We would like to use the miRNA to disrupt the propagation of the infectious virus. In instances where other groups (Lagos-Quintana, et al, (2001), Lau, et al, (2001), Zeng and Cullen (2002), Zeng, et al, (2002), Zeng and Cullen (2003), Lim et al, (2003), Zeng, et al, (2003), Zeng and Cullen (2004), Lim, et al, (2005), Creighton, et al, (2010)) have used existing miRNAs to achieve results, we describe herein a method to create miRNA that would be directed against the enzyme of interest. First, we want to select sites within the gene of the enzyme that would provide a good target for the miRNA to bind, with the stipulation that the linear stretch would be maximally conserved within the gene of the enzyme.

Next we find a sequentially conserved stretch of nucleotides of the enzyme of interest. For this purpose, we identify a conserved stretch (22-24 nts) within the gene of the enzyme that could lead to inactivating the enzyme. One of the sequences for the enzyme was obtained from one of the public databases. This sequence was used to pull all the nucleotide sequences from the National Center for Biotechnology Information (NCBI) (www.ncbi.nlm.nih.gov) using “blastn” (Altschul, et al, (1997), Stephen et al., (1997)) against “Genbank (Benson et al., (2003)), EMBL (Stoesser et al., (2003)), DDBJ (Tateno et al., (1998)), PDB (Berman et, (2000))” sequence databases. A separate program was written to collect all the sequences from the blast output with high sequence identity (>80%) to the query sequence and a length greater than 90% of the query sequence to get a multiple sequence alignment (MSA), maintaining the alignment from the blast run. Another script was written to analyze the multiple sequence alignment file to calculate the frequency of occurrence as a percentage for each nucleotide at each location. This analysis provided stretches that are maximally conserved for a given length of the stretch.

A new nucleotide sequence for the enzyme is created with nucleotides that have the highest frequency at each location. For each position along the length of the sequence, the nucleotide having the maximum frequency as well as its frequency is stored. Using this, we calculate the cumulative frequency for a given window of nucleotides (22 to 24) at every location and are sorted in descending order of their scores of cumulative frequencies. Next, the enzyme protein structure is analyzed visually using “PYMOL” graphics program (PyMOL™ Evaluation Product—Copyright (C) 2008, DeLano Scientific LLC). The amino acids corresponding to those stretches are viewed to determine if they constitute the active site or support the structure of active site. A reverse complementary sequence (antisense) to this stretch of ˜22 nucleotides would define the miRNA.

Protection of the TV from the Designed miRNA.

Because the genome of the TV is modeled from the infectious virus, the designed miRNA against the infections virus may be expected to disrupt the production of the TV. To avoid this case, the binding stretch within the genome of the TV is modified to have a different sequence of nucleic acids. As described earlier, the sequence modifications could be guided by the MSA, or they could be modified manually so that the changes are greater at the nucleic acid level and small at the amino acid level. Through modification at the nucleic acid level, the lack of significant disruption of protein folding, ensures that the miRNA designed against a stretch in the infectious virus will not disrupt the post-transcription processing of the TV.

Design of Pre-miRNA for the Antisense miRNA.

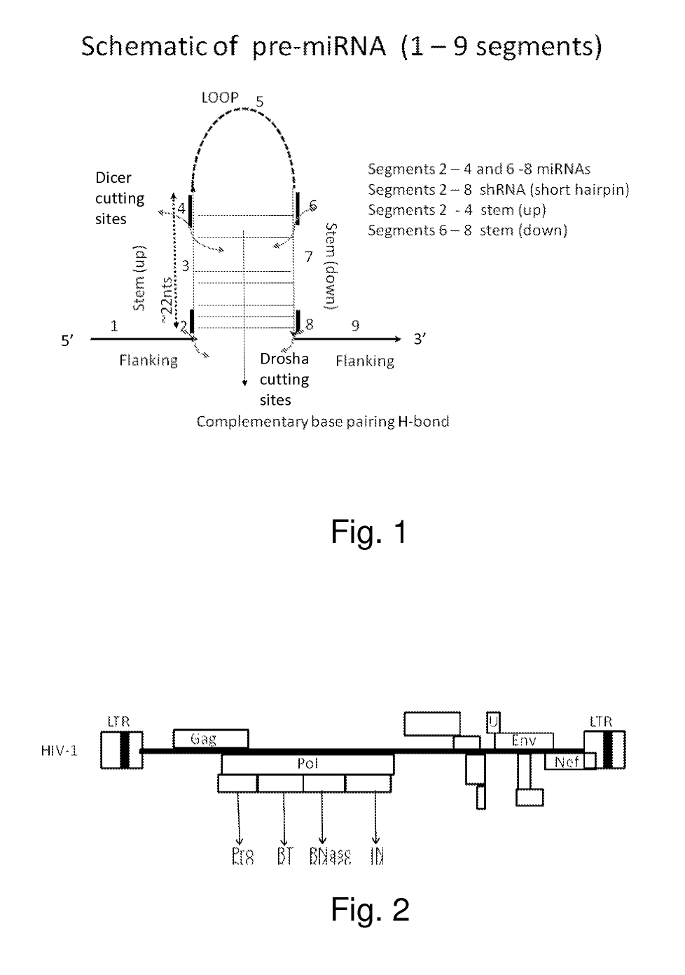

The rationale for this design follows. We have seen that, enzymes dicer and drosha play their roles in the release of the miRNA from the pre-miRNA. In this design we provide a natural environment for enzyme cleavage to take place at the expected site. We search the miRNA database to find miRNAs that have maximum number of identical nucleotides at the 3′ end to the designed miRNA. These hits would be used to design the loop (segment 5) and segment 6 of the pre-miRNA (FIG. 1) to resemble L-sshRNA. Similarly, by selecting miRNAs from the database having maximum number of nucleotides matching with the 5′ end of the designed miRNA, we would design the segments 1,8, and 9 of the pre-miRNA. In this way, we would have a stretch of nucleotides flanking the scissile bonds of our pre-miRNA that have been observed in the database. This strategy provides the best opportunity to observe the cleavage by the enzymes at the expected site.

The miRNA could be in either stem(up) or stem(down) orientation. The following description corresponds to designing miRNA in the stem(up) (L-sshRNA) orientation. From the mirbase database, we identify miRNAs that have a relatively high number identical nucleotides (at least three nucleotides) at their 3′ end matching the nucleotides at the 3′ end of the designed miRNA (antisense). If the corresponding shRNA of the database miRNA has its loop at the 3′ end (L-sshRNA orientation), then we select this miRNA and its corresponding shRNA. For the design of pre-miRNA, we use the loop sequences (for segment 5) and the part that is opposed to those matching nucleotides at the 3′ end (segment 6). This analysis can include inspection of the secondary structure of the shRNA using any program that would predict the possible secondary structures of nucleotides and we have used the program RNA structure (see, e.g., Reuter and Mathews (2010)). This design would give an environment that is observed in the database for the dicer to cleave at the expected site.

We search the miRNA database (mirbase) (Ambros, et al, (2003), Griffiths-Jones (2004), Griffiths-Jones, et al, (2006), Griffiths-Jones, et al, (2008), Saini, et al, (2008), Meyers, et al, (2008), Kozomara A and Griffiths-Jones S (2011)), to find mature RNA sequences that have maximum number of nucleotides (>3 nucleotides) at their 5′ ends matching nucleotides at the 5′ end of the designed miRNA. We find the corresponding shRNAs from the mirbase related to these miRNAs, and select the one (designated mir_{—}1) with the most matching sequences and the mature RNA as stem(up) as opposed to stem(down) in the shRNA. Next we use this shRNA (designated hairpin_{—}1) as a query and search the nucleotides database (using a program like BLAST) to get the primary transcript (mir_{—}1_primary) of hairpin_{—}1. Now, we identify the position of the shRNA within the mir_{—}1_primary and extend ˜65 nts on either side to get the flanking sequences.

The matched sequence at the 5′ end of the designed antisense with the selected miRNA from the database, is considered to be second of the nine segments. The flanking sequences (˜60 nts) from the 5′ end of the antisense miRNA primary transcript mir_{—}1_primary will be the first segment. The flanking sequences (˜60 nts) from the 3′ end of the antisense miRNA primary transcript mir_{—}1_primary will be the ninth segment.

Similarly, we find a mature RNA (mir_{—}2) from the mir database which has maximum number of nucleotides (>3 nucleotides) at their 3′ end matching with the 3′ end of our designed antisense. This part of the 3′ end nucleotides of the designed antisense is the fourth segment of the nine segments. We find the shRNA that contains this mir_{—}2 (named hairpin_{—}2) and predict the secondary structure using a program such as RNA structure (Reuter and Mathews (2010)). We can identify the loop sequences, which form the fifth segment of the nine segments.

Now, from the secondary structure prediction, we identify those sequences that correspond to the 3′ end matched sequences in the hairpin_{—}2 (fourth segment) and this will be the sixth segment. Sometimes, segment 4 and segment 6 may not be 100% complementary.

Those nucleotides in the designed antisense that are not part of either segment 2 or segment 4 will be segment 3. Now we develop a perfect complementary sequence for this segment 3 in reverse order and name it segment 7.

Similarly, we also identify those sequences in hairpin_{—}1 that correspond to the matched sequences at the 5′ end (segment 2) and name it segment 8.

Combining segments 1 through 9 will give us the pre-miRNA that contains the designed antisense as a stem(up) sequence.

The following description is for the case where we want the designed antisense to be the stem(down) part of the shRNA or in the R-sshRNA according to the definition of (Khvorova, et al, (2003), Ge, et al, (2010)). We search the mirbase to find mature RNA sequences with a maximum number of nucleotides (>3 nucleotides) at their 5′ ends matching with nucleotides at the 5′ end of the designed miRNA. The miRNA (named mir_{—}3) is found from the mir-database. We find the corresponding shRNA for selection if the mature RNA is stem(down) in the shRNA. We find the shRNA (named hairpin_{—}3) that contains this mir_{—}3 and predict the secondary structure to identify the loop sequences that form the fifth segment of the nine segments.

The matched sequence at the 5′ end of the designed antisense with the selected miRNA from the database is considered the sixth of the nine segments. From the secondary structure prediction, we identify that part that is opposed to the sixth segment as the fourth segment. Sometimes, segment 4 and segment 6 may not have 100% complementarity.

Similarly, we find a mature RNA (mir_{—}4) from the mirbase which has maximum number of nucleotides (>3 nucleotides) at their 3′ end matching with the 3′ end of our designed antisense. This part of the 3′ end nucleotides of the designed antisense is the eighth of the nine segments.

We find the shRNA (named hairpin_{—}4) that contains this mir_{—}4 and predict the secondary structure. From this prediction, we identify those sequences that correspond to the 3′ end of the matched sequences (eighth segment) in the hairpin_{—}4 to be the second segment.

Those nucleotides in the designed antisense that are not part of either segment 6 or segment 8 will be the segment 7.

Now we develop a perfect complementary sequence for this segment 7 in the reverse order as segment 3.

We use the hairpin_{—}4 to search the nucleotides database (using programs like BLAST) for the primary transcript (mir_{—}4_primary) of the hairpin_{—}4. From this analysis we can extract the flanking sequences of desired length (˜60 nts long) that are in the 5′ as well 3′ ends of the shRNA (hairpin_{—}4). Then we extract flanking sequences of desired length from 5′ end of the hairpin_{—}4 within mir_{—}4_primary to be the first segment.

Similarly, we extract flanking sequences of desired length from the 3′ end of hairpin_{—}4 within the mir_{—}4_primary to be the ninth segment.

Now, combining all the segments one through nine will give us the pre-miRNA that would contain the designed antisense as a stem(down) sequence.

### Details of the Methodology.

We have applied the generic methodology above to design a specific TV against HIV-1 infection.

The schematic view of the HIV-1 genome (FIG. 2) describes the different gene products of the HIV-1 genome; a more detailed description is given at http://hivinsite.ucsf.edu/ under “Comprehensive, up-to-date information on HIV/AIDS treatment, prevention, and policy from the University of California San Francisco”, Thomas J. Hope, PhD, Didier Trono, M D from The Salk Institute. This genome codes for proteins such as Gag and pol which contain Rev-Responsive elements, matrix, capsid, and nucleocapsids. The gene encoding pol (polypeptide) codes for different proteins/enzymes such as protease, RT, RNase, and integrase, which are individually produced after cleavage by the protease. Additionally, the HIV-1 genome has long trailing repeats called LTRs on either side.

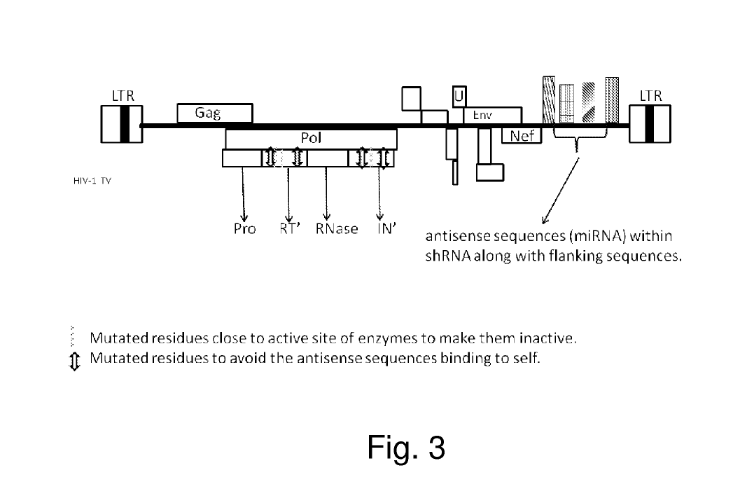

The HIV-genome codes for a few enzymes that are essential for its activity. The HIV-1 genome can be modified (FIG. 3) so that any progeny virus is rendered noninfectious. This redesigned virus can be achieved by modifying some of the residues of the enzymes that are important for their activity by site-specific mutations, or by completely removing the particular gene. In this invention, we have chosen to inactivate, the reverse transcriptase and the integrase. Apart from these mutations, stretches of nucleotide sequences that are relatively highly conserved within the gene encoding the RT are modified to have a different nucleotide sequence relative to the corresponding ones in the infectious virus. These sites from the infectious virus are the target for the antisense sequences. We select the conserved stretch of nucleotide sequences from RT since the three dimensional structure is known. Per our design strategy, the corresponding nucleotides in the therapeutic virus are mutated to have different sequences. Selecting the stretches based only on the sequence information would not let us know how to modify them without affecting the three- and four-dimensional nature (packing of the different proteins) of the system or to know the implications of deleting it.

Next, we deliver the RNAs of the antisense from within the infected cells. We design pre-miRNA sequences and insert them within a UTR region of the designed virus, making them part of the TV. As explained earlier, dicer and drosha enzymes within the host cells would release the miRNAs from the hairpin structures that are part of the pre-miRNAs. Thus, the miRNAs are produced within the cell from its genome once it is incorporated into the host genome. More importantly, we would expect this to inhibit translation of partially complementary mRNA, which is described as a characteristic of miRNAs.

Another aspect of this design is to allow the genome of the TV to be incorporated into the host genome already infected by infectious virus. We expect that enzymes such as reverse transcriptase and integrase from the infectious virus would be available to enable the initial steps of incorporating the TV into the host genome. Accordingly, the antisense sequences would be generated with every cell cycle. We preserve the other sequences of the virus genome (e.g., LTRs, capsids, nucleocapsids, proteins, glycoproteins, REV-responsive element, etc) so that the packing of this TV would resemble the infectious virus externally, maintaining the specificity and selectivity towards those cells targeted by the infectious virus. The TV will permeate into any cell type that could be infected by the infectious virus, as well as become incorporated into the host genome of those cells already infected by HIV-1, continuously producing the antisense sequences to silence the essential enzymes of the infectious virus.

The resulting TV is envisaged to help fight the HIV virus from within, once the therapy begins. This strategy specifically blocks the process of making essential enzymes of the infectious virus by the host cell. In principle, the idea can be used to deliver antisense sequences against any of the viral enzymes, such as the protease, integrase, RNaseH or RT, even though we describe antisense only against the reverse transcriptase of the HIV-1.

For the TV to be transcribed, RT must be present in the cell. Therefore, TV transcription is possible only in cells that have already been infected by the infectious HIV-1 or another retrovirus. Once the TV is reverse transcribed by the reverse transcriptase and integrated into the host genome, RT is no longer needed. This issue is addressed by the development of miRNA against the RT gene. Once the TV is incorporated into the host genome, it would produce more TV as well as miRNAs that will intervene in the translation process of the RT of the infectious virus. Thus, this process allows the TV to incorporate only into the host cell genomes that have are already infected by HIV with copies of RT available. Preferably, we do not inhibit the protease since this enzyme is required to package the TV within the cell.

Most of the existing HIV therapies fail due to development of resistance as the HIV mutates. With small molecule inhibitors of enzymes, a mutation of one residue close to the active site may change the active site and prohibit the inhibitor from binding. With the present invention, the intervention is at the pre-translation step and mRNA function would not be expected to be impacted adversely by one or two mutations. Thus developing viral resistance to this therapy is expected to be minimal. Additionally, with the possibility of more than one antisense sequence directed against same enzyme or multiple enzyme targets of the viral genome, resistance would require mutations in all the segments that contain the antisense sequences. The probability of this process occurring is very low.

The technology described here allows for the host cell to produce copies of the modified virus by itself, so that there may be no need for continuous administration of the therapy. Once we coinfect an already infected HIV cell with this TV, the process of making the antisense sequences will continue until all production of infectious virus is stopped. The replication process of the virus is error prone by nature, and introduction of random mutations is common. This method could fail at the stage when there are mutations in either the RNA sequence targeted by antisense sequences or by mutation at the antisense sequences itself in the TV. However, this concern is minimized by having more than one antisense sequence to bind to different sites of the RNA of the same enzyme.

Recombination is another aspect to be considered. We believe that the complete removal of the RT gene from the therapeutic virus may reduce the chances of converting the TV to an infectious agent. Rather than coming up with an infectious therapeutic virus by acquiring the RT or the integrase gene from the infectious virus genome in the process of recombination, the RT or the integrase would be destroyed by the TV encoded antisense. Complete removal of either the RT or integrase gene from the TV genome could also change the packing of these enzymes within the therapeutic virus and cause other problems.

### EXAMPLES

The following examples are offered to illustrate, but not to limit the claimed invention.

It is understood that the examples and embodiments described herein are for illustrative purposes only and that various modifications or changes in light thereof will be suggested to persons skilled in the art and are to be included within the spirit and purview of this application and scope of the appended claims.

### Example 1

### Designing a TV Against HIV-1

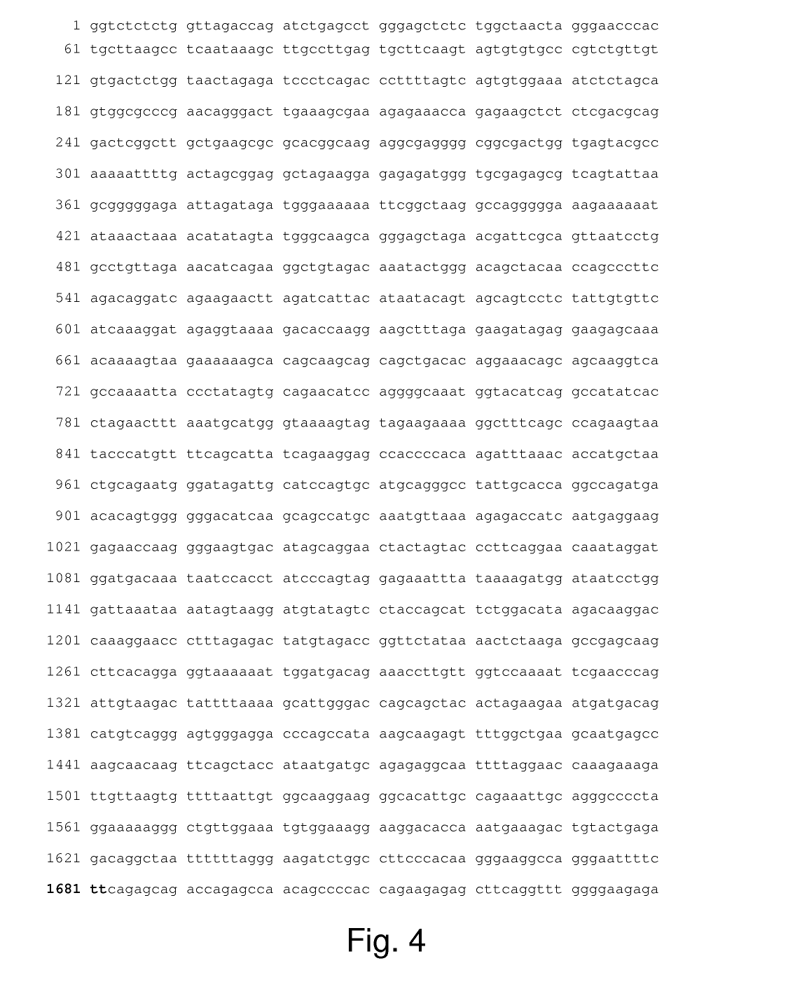

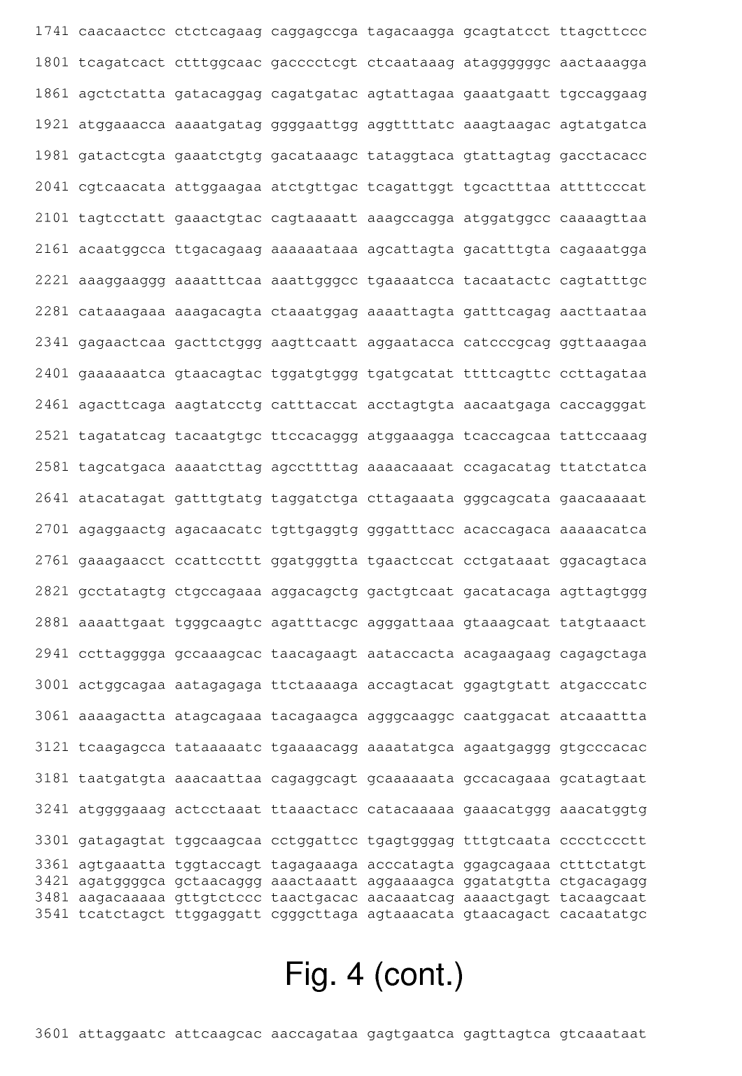

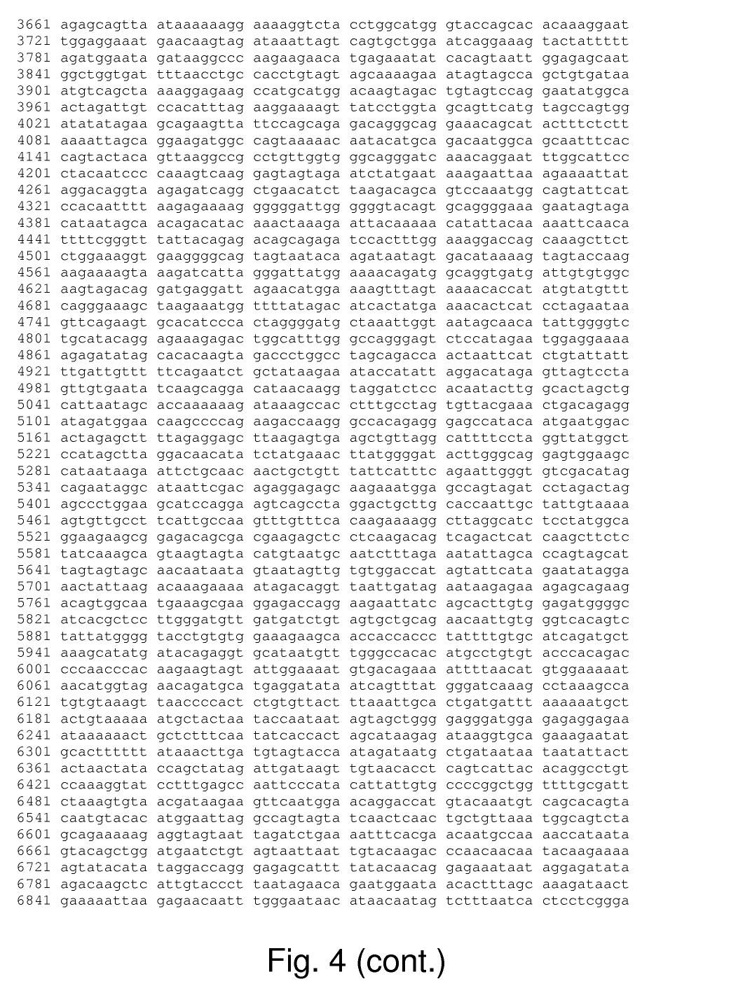

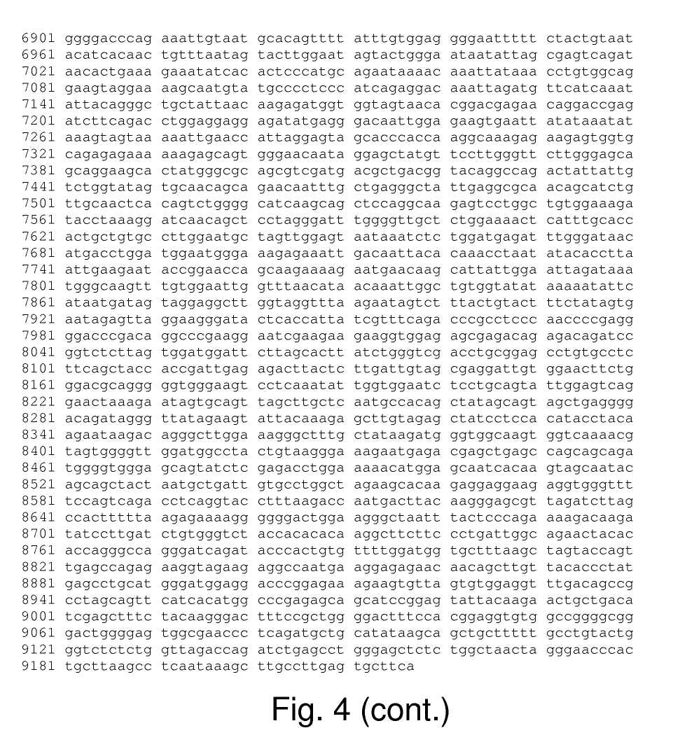

Though many sequences are available in the Genbank database, e.g., starting from (U26942), Adachi, et al, (1986), Salminen, et al, (1995), we use a genomic sequence (Fang, et al, (2001) (gb: U69584.1) of HIV-1 (FIG. 4) as a reference for our further modifications. Any full-length HIV-1 sequence could be used for the experimental purposes.

### Active Site Modifications.

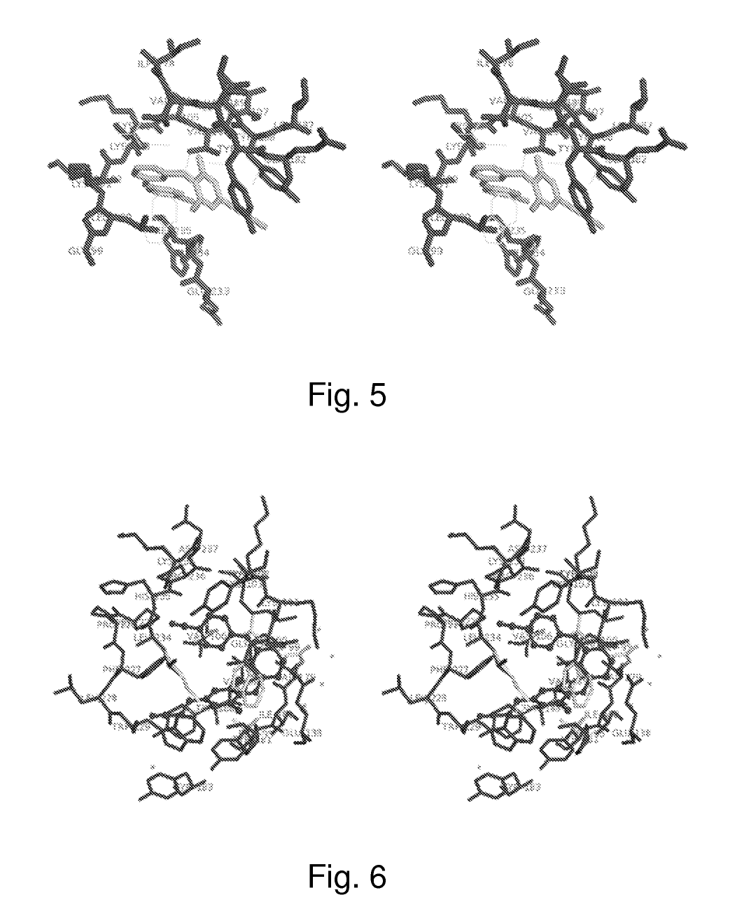

The active site of 2zd1.pdb with some of the residues suggested for mutations to inactivate the enzyme highlighted is shown in FIG. 5. Mutations L100W (TTA->TGG), K103Q (AAA->CAG), V106E (GTA-GAG), V179E (GTT->GAG), Y181T (TAT->ACC), Y188L (TAT->CTC) and L234K (CTC->AAG) are expected to inactivate the RT enzyme. These residues are involved with non nucleosides reverse transcriptase inhibitors (NNRTI). The mutated residues are shown in thin gray lines. The mutations are expected to interfere with the substrate binding. The ligand is in light color in sticks.

Similarly, another structure 1rtd.pdb (Huang, et al, (1998)) was used to identify residues in the active site involving nucleoside reverse transcriptase inhibitors (NRTI), for directed mutagenesis to inactivate the enzyme. FIG. 6 shows the suggested mutations (K65S-AAA->TCT; R72I-AGA->CTT; D110E-GAT->GAA; P150A-CCA->GCA; Q151F-CAG->TTT; D185T-GAT->ACA; D1861-GAT->ATA) (in cross-eye stereo) based on 1rtd.pdb. The mutated residues are shown in light color. Mutation K65 is known to interact with the incoming substrates. So we have changed it to a serine (AAA-TCT). Mutation P150A is known to inactivate the enzyme (Smith, et al, (2006)) (CCA to GCA). Q151K is also known to inactivate the enzyme (Matsumi, et al, (2003)). Similarly, Q151V leaves the enzyme with a relative activity of 4.3% compared to the wild type (Smith, et al, (2004)). From structure analysis, we preferred to use the mutation of Q151F (CAG to TTT). Similarly, we also suggest that the mutation R72I would exploit the loss of a favorable interaction with R72, while the mutated F151 and the 172 will interact favorably, as both are hydrophobic residues. Smith, et al, (1999) has indicated the possibility of mutating P157 to serine which is also naturally occurring and resistant to nucleoside analog therapy. We suggest to eliminate it, so that the loop is shortened and the chances of acquiring the original sequence would be extremely low. Smith, et al, (2006) also reported that mutations at K154 mostly inactivate the enzyme and at least one clone with this mutation was found, although the infectivity of this mutant protein compared to the wild type was not determined. We propose to eliminate residue 154. Also, Julias, et al, (2001, 2004) have reported that D110E inactivates the enzyme. Mutation of D186 has a profound effect in inactivating the enzyme (Kaushik, et al, (1996)) and hence, we have made D186I to inactivate the enzyme with maximal codon differences between the two (GAT to ATA). Similarly, D185 is changed to a threonine (GAT to ACA). Apart from these changes, G155 is also eliminated so that the loop containing this segment will be shortened in the TV, so that the antisense would not bind to this site. In another embodiment of this invention, we removed the nucleotide codings for the RT enzyme of the TV genome, against which the antisense miRNAs have been developed. In this case, the recombination would be tolerated.

Selection of the First Conserved Site within RT for Creating Antisense Sequences.

When selecting sites for creating antisense sequences, we wanted to know if the loss or breakage at those sites would render the enzyme inactive in the infectious virus and how modifications to that site would affect the folding of the structure in the TV. These two aspects are critically important in our design. When we select a site for creating the antisense, the antisense sequence would bind to that site in the mRNA rendering the produced protein incomplete. We want to know if this incomplete structure is inactive so that the infectious virus would be innocuous. Further, we need to ensure that modification to a particular site will not interfere with incorporation into the TV. These modifications serve two functions. One, the antisense developed against that site will not bind and affect the TV. Second, for targeting the cells concerned, we want the TV to resemble the infectious virus externally by having similar viral packing.

The next step was to find a sequentially conserved stretch of nucleotides within the gene for RT. For this purpose, we plan to find a stretch (of length 22-24) within RT, relatively more conserved than other stretches and structurally meaningful in inactivating the enzyme. One of the sequences for the RT was obtained from a public database. It was used to gather all nucleotide sequences from the National Center for Biotechnology Information (NCBI) (www.ncbi.nlm.nih.gov) using “blastn” (Altschul, et al, (1997), Stephen et al., (1997)) against ““Genbank (Benson et al., (2003)), EMBL (Stoesser et al., (2003)), DDBJ (Tateno et al., (1998)), and PDB (Berman et, (2000))” sequence databases.

Using as an example, the nucleotide sequence for HIV RT is used to “blastn” against all the nucleotide sequences in NCBI site. We have used the word blast as a verb to denote running the BLAST program. To get a multiple sequence alignment and maintain the alignment from the blast run, a separate program was written to collect all the sequences from the blast output with high sequence identity (>80%) to the query sequence and with a hit length at least more than 90% of the query sequence. 4944 sequences were identified. Another script was written to analyze the multiple sequence alignment file to calculate the frequency of occurrence as a percentage of each nucleotide at each location. That percentage is given a null value if that location does not have a significant number of nucleic acids in the total number of sequences. These numbers are used to identify stretches that are maximally conserved for a given stretch length.

A new nucleotide sequence for the RT is created with nucleotides that have the highest frequency at each location. For each position along the length of the sequence, the nucleotide having the maximum frequency as well as its frequency is stored. Using this we calculate the cumulative frequency for a given window of nucleotides (22 to 24) at every location and are used to find the stretches of nucleotides having higher cumulative frequencies. Next, the RT enzyme structure is visually analyzed using the “PYMOL” program (PyMOL™ Evaluation Product—Copyright (C) 2008, DeLano Scientific LLC). The amino acids corresponding to key stretches are analyzed regarding their impact on the active site and in turn enzyme structure and function. Once we have identified the site, with a length of ˜22 nucleotides, an antisense sequence is obtained for the same.

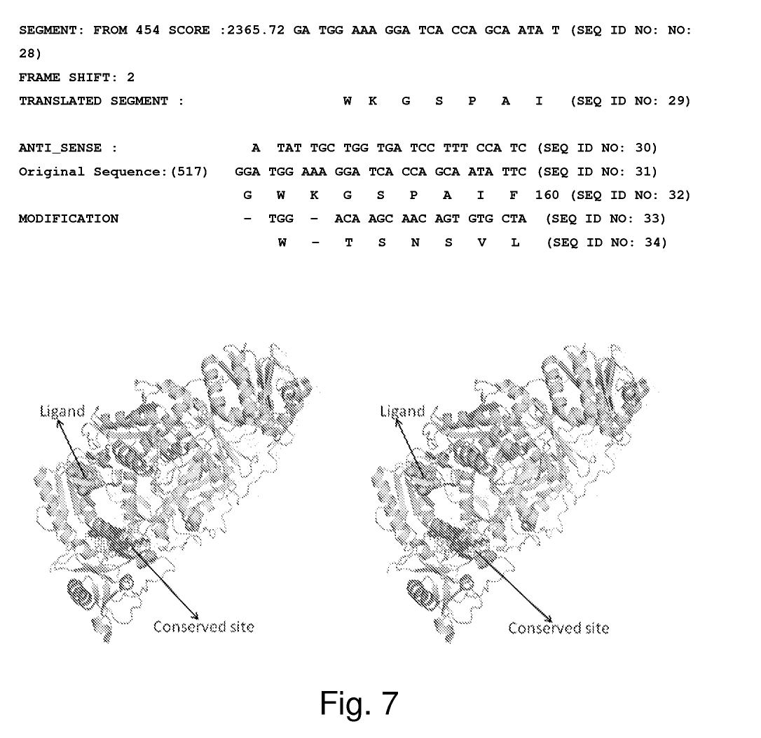

In FIG. 7 we detail the top scoring conserved stretch of nucleotide sequence for RT, including the segment starting number, the score for the stretch, and the sequence of these 24 nucleotides. The amino acids corresponding to this segment are also given. In the next line we provide the antisense for the segment. The location of the stretch within the 2zd1.pdb in the context of the active site is seen in FIG. 7. This conserved segment starts at residue W152. Part of the active site is formed with residues V179, I180, and Y181 which follow directly after the above segment.

If antisense were to bind to WKGSPAI, then the part that forms the active site will be lost and the enzyme will be inactive. Note that this segment is derived from the infectious HIV virus. If we use the antisense in our noninfectious TV, the antisense sequence would bind to them also and disrupt the propagation of the TV. We envision at least two ways to avoid this occurrence. One, we remove the gene encoding the RT from its genomic sequence. Two, we mutate those amino acids of the conserved segment in such a way that the mutated residues have maximum dissimilarity in terms of the triplet codons to the selected segment without greatly perturbing the 3D structure. In this way, we avoid binding of the antisense to our TV.

For the selected segment for RT, we have tried to shorten the loop by deleting G152 and K154. Certain other mutations, e.g., G155T and P157N are based on visual analysis of the structure. We wanted to change G155, because the among previous residues, W153 is encoded by TGG. The 2 guanosines will give rise to a guanosine repeat (G-quartet which have been implicated in recombination of RNAs) (Shen, et al, (2009)), if G155 (GGA) were not modified. There are examples for the Pro to Ser mutation (from the structure AY358072), so we believe P157N would be plausible. The Ala to Ser is from AY779556.

For the amino acid I159, which is surrounded by hydrophobic residues, there are few sequences with either a threonine (gb|AY560444.1, gb|AY275555.1, gb|AY560412.1) or valine (gb|AY331286.1, gb|AY331285.1, gb|JF689897.1). We have chosen to use GTG, which codes for a valine. Next, the mutation from Phe to Leu is from EU489663. Along with the above mentioned changes to the selected conserved segment of RT, we also suggest to delete the G152 (GGA) and K254 (AAA) and to use a different codon for the next serine. The alanine residue has been changed to a serine (GCA to AGT). Thus, the very segment which is part of a loop in the RT structure is shortened, making it very difficult for the designed miRNA to bind to this site. Modeling the conserved segment after the deletion of the two residues G152 and K154 did not show any great change in the structure of the loop, so we believe that changes may not affect the structure significantly. The sequence of RT after incorporation of these changes is termed modified sequence RT′. The gene equivalent to the RT of the infectious virus in the TV will have the modified sequence RT′ at the corresponding site.

Several miRNAs, their shRNAs, and their precursor pre-miRNAs have been documented, and a database has been created (mirbase) (Ambros, et al, (2003), Griffiths-Jones (2004), Griffiths-Jones, et al, (2006), Griffiths-Jones, et al, (2008), Saini, et al, (2008), Meyers, et al, (2008), Kozomara A and Griffiths-Jones S (2011)). An analysis of the miRNA database gives an idea on the location of the matured miRNA within the hairpin sequence. The antisense miRNA has been found to be either of the two stem sequences or in some cases both the stem(up) and stem(down) sequences. We design our antisense to be in stem (up) orientation (L-sshRNA). The following are the steps in the construction of the pre-miRNA for the antisense sequences.

To start our construct, we selected those mature RNAs (species Homo sapiens) from the database (mirbase) that have maximum identity at the 3′ end to our antisense sequence. For example, the antisense sequence for the first segment in RT (ATATTGCTGGTGATCCTTTCCATC (SEQ ID NO: 11) given in FIG. 7) ends with TCCATC. One of the mature RNA from the miRNA database given by MIMAT0019885 (TGCGGGGACAGGCCAGGGCATC (SEQ ID NO: 12)) also ends with CATC. If the mature sequence MIMAT0019885 in its shRNA hsa-mir-4749 happens to be on the 5′ side of its loop sequence (stem(up)), then that loop structure would be used as the loop for our pre-miRNA construct. Similarly, if the miRNA MIMAT0019969 (ATATTATTAGCCACTTCT GGAT (SEQ ID NO: 13)) from the database that matches the antisense of our construct at the 5′ end (ATATT) (the shRNA would be has-miR-4795_{—}3p) were to be in stem(up) orientation, then it would be chosen for selecting both 5′ end and 3′ end flanking sequences. However, the two sequences from the miRNA database were in the stem(down) orientation, so we had to explore different lengths of the antisense.

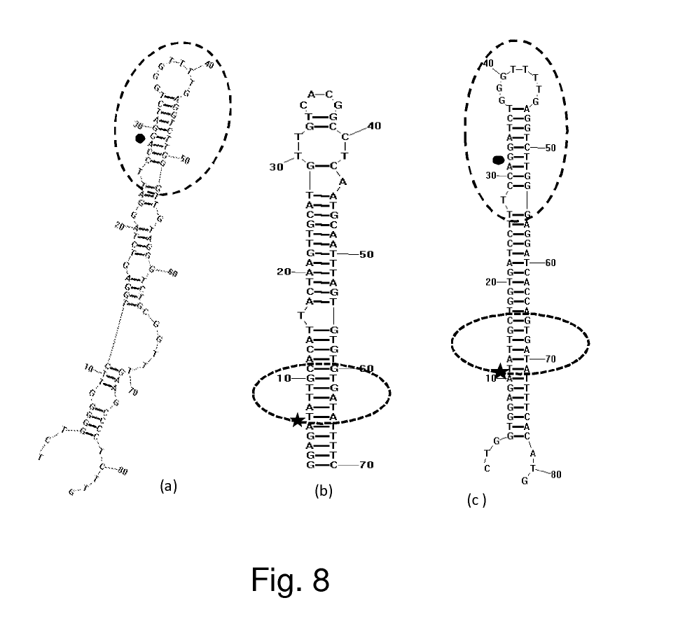

When we used a 22 nt antisense against the first conserved segment for RT, many of the hits from the miRNA database had the matured RNA (miRNA) in the stem(down) orientation; they were on the 3′ end of the loops. So we had to settle with a 21 nucleotide stretch that would find matured miRNAs to the 5′ end of the loop. The final antisense chosen is “TATTGCTGGTGATCCTTTCCA (SEQ ID NO: 14)”. For this sequence, we had the best hit “CTGGAGTCTAGGATTCCA (SEQ ID NO: 15)” for the 3′ end from MIMAT0016859, which is from the hairpin sequence hsa-miR-4309. FIG. 8a shows the predicted secondary structure of hsa-miR-4309 using the program RNA structure (Reuter and Mathews (2010)), of the hairpin RNA. From this picture, we predicted the loop sequences by following the stem-loop-stem construct for the hsa-mir-4309 hairpin sequence.

Similarly, for the 5′ end, we chose the miRNA MIMAT00000090 with the maximum matches with our antisense at the 5′ end “TATTGCACATTACTAAGTTGCA (SEQ ID NO: 16)”, which lies in hsa-miR-32 and further, this miRNA is stem-(up) within the hsa-mir-32 as shown in FIG. 8b. We used the hairpin sequence of hsa-mir-32 as query sequence and used the program “blastn” to get the flanking sequences. In this sequence, we have replaced both the stem sequences (stem up and stem down) with the antisense sequence and the sense sequence maintaining the flanking sequences and the loop sequence. Noteworthy, the stem-down sequence could be modified slightly to suit the target sequences for obtaining the loop and the flanking sequences while the stem-up sequence is maintained per our design. Because of this manipulation, the stem-up and stem-down may not be exact complements. For example, in FIG. 8c, there is no complementary nucleotide for nucleotide 28 (T), present to maintain the environment as seen in the shRNA (hsa-mir-4309) obtained from the miRNA database. In the same way, at the stem(down) sequence, we deliberately assigned some non-complementary nucleotides to the stem(up) sequence. These modifications to stem(down) sequences are limited to the matching number of nucleotides at the 3′ end and 5′ end of the antisense (i.e, to the stem(up) sequence), which is maintained as designed.

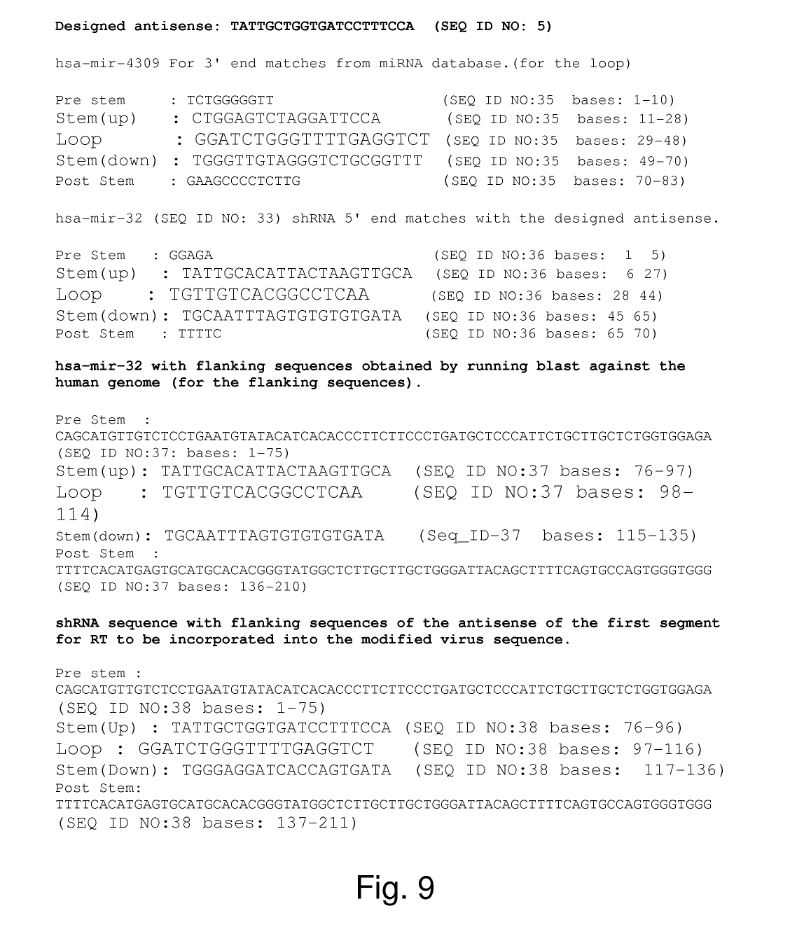

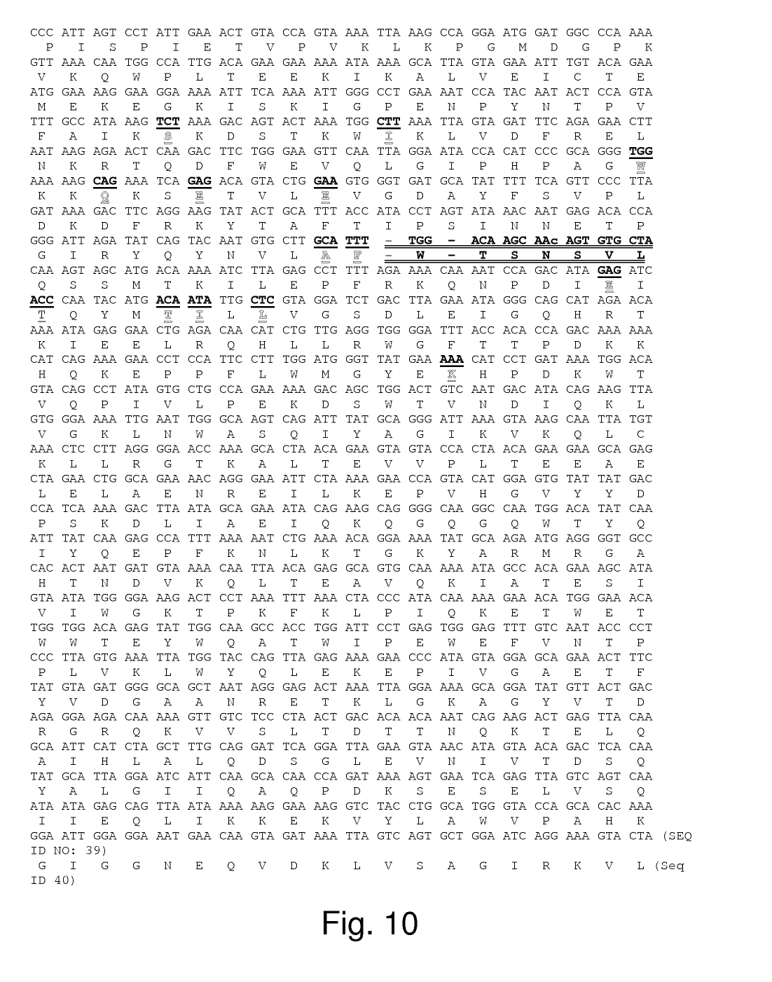

FIG. 9 shows the steps involved in developing the pre-miRNA for the first segment of the RT-miRNA hit with flanking sequences derived from the miRNA hairpin sequence with its flanking sequences. This figure shows the loop sequences chosen from the hsa-mir-4309, the flanking sequences from hsa-mir-32, and also minor modifications to stem-(down) sequence as explained previously.

FIG. 10 details the nucleotide sequence for RT1, which has modifications to the consensus sequence of inactivated RT as well as the changes to the first conserved site against which the antisense miRNA was developed.

Selection of the Second Site within RT for Creating Antisense Sequences.

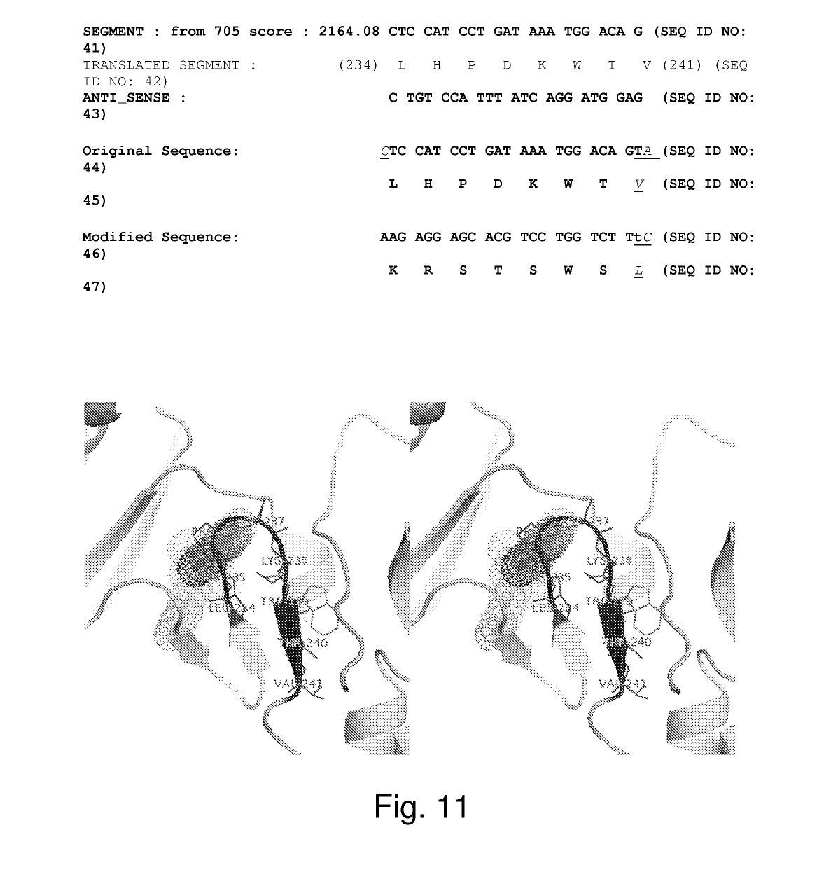

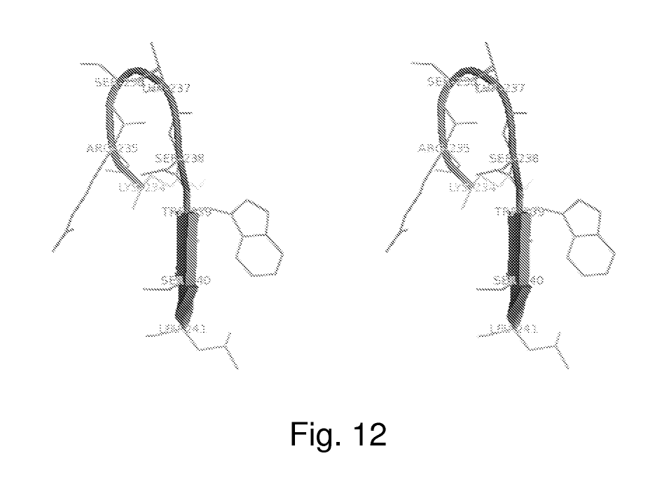

The next high scoring conserved segment within the RT gene was evaluated for designing another miRNA as described previously. FIG. 11 gives the sequence information for the second segment, along with the structure of the corresponding segment within the enzyme structure (2zd1.pdb). The ligand is shown as dotted spheres.

FIG. 12 shows the suggested mutations (as modeled) to this segment of residues to avoid the self binding of antisense developed against this second site. These mutations were based on either visual inspection of the particular site or from other reverse transcriptase sequences (homologs or orthologs) using sequence alignment.

The mutation V106E was based in the context of the mutation L234K to inactivate the enzyme. This mutation is expected to establish a salt bridge between V106E and L234K. The H235R mutations is observed in AY560487; P236S in AF331209; D237T in GU328920; K238S in AB253431; T240S in GU345227; and V241L in AF289548. We use a different triplet for the different amino acids observed in other proteins to maximize the difference between this new set of nucleic acids compared to the antisense created against the second site. The segment has a sequence CTCCATCCTGATAAATGGACAG (SEQ ID NO: 17), with its antisense being CTGTCCATTTATCAGGATGGAG (SEQ ID NO: 18). For this antisense, the sequence that matches at the 3′ end is MIMAT0019728 (TGCAGCTCTGGTGGAAA ATGGAG (SEQ ID NO: 19)) and its shRNA is hsa-mir-4660. Similarly, the mature RNA given by MIMAT0009979 (CTGTAATATAAATTTAATTTATT (SEQ ID NO: 20)) (the shRNA being hsa-miR-2054) has matching 5′ end sequences, which are used here for designing the flanking sequences.

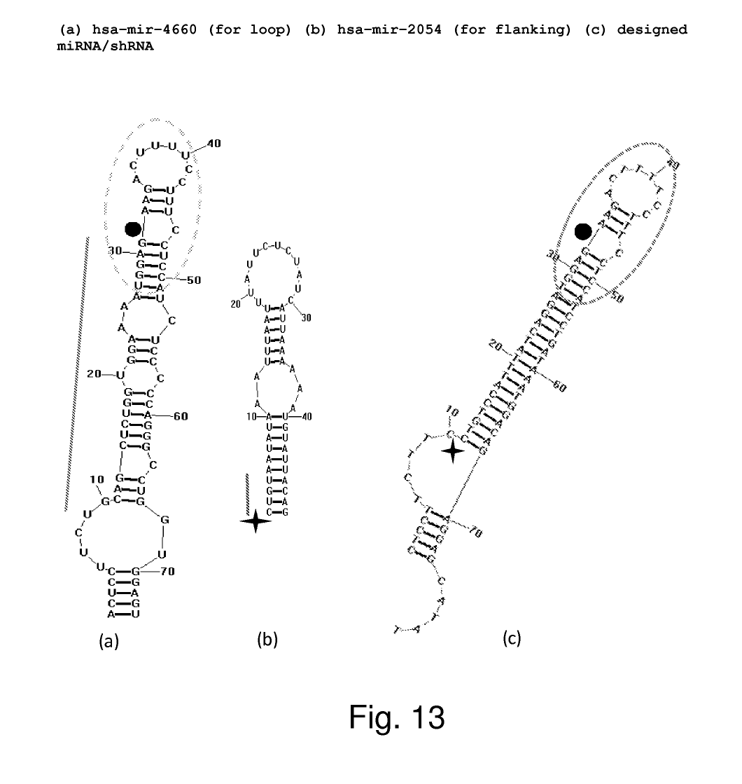

FIG. 13 details the predicted secondary structures for the hit from the miRNA database, (a) (hsa-mir-4660) for loop selection, (b) the miRNA hit for the flanking sequences (hsa-mir-2054), and (c) the shRNA for the designed antisense combining the loop structure from the hsa-mir-4660 and the short flanking from the shRNA of hsa-miR-2054. The putative scissile bond for dicer is indicated by a black dot and that for the drosha by a black star.

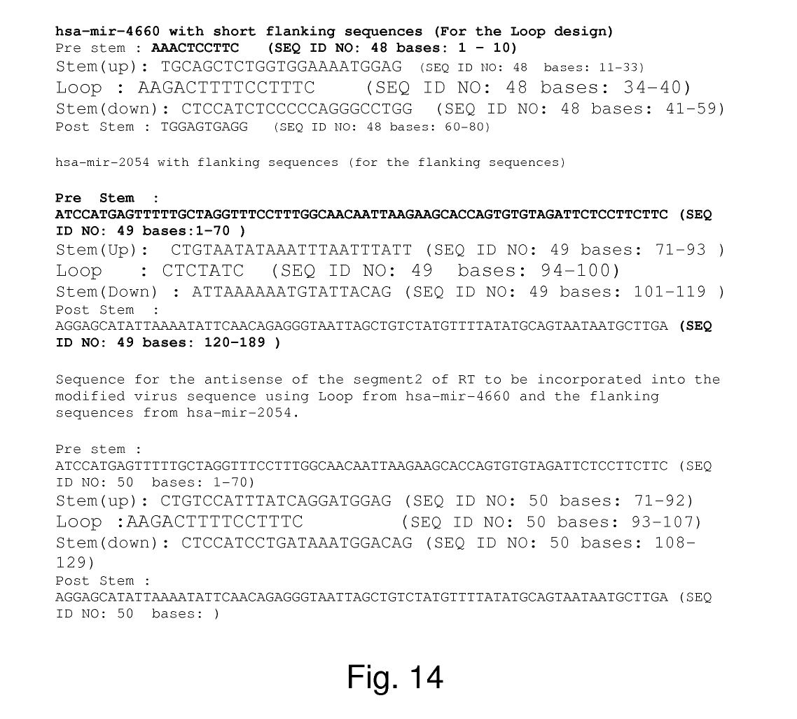

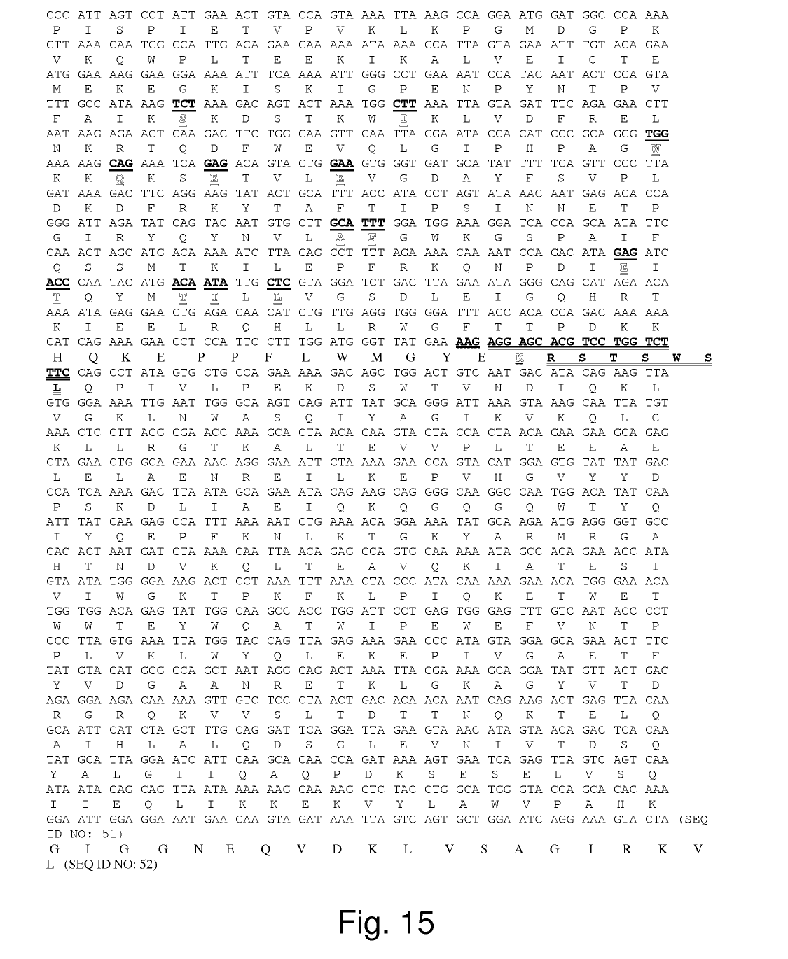

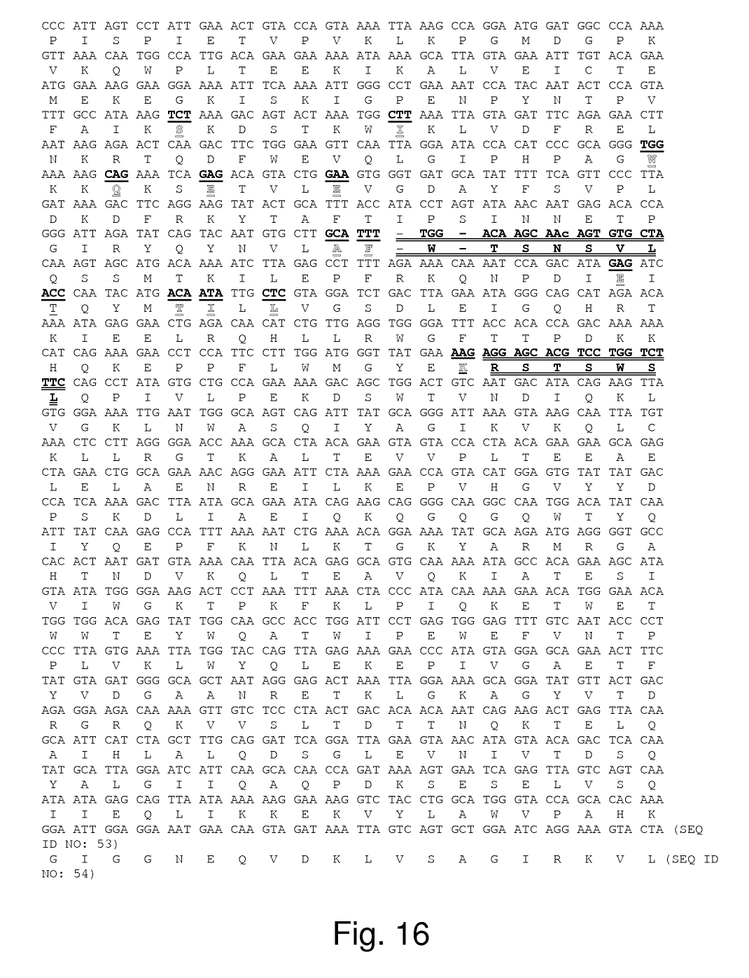

FIG. 14 shows the construction of pre-miRNA containing the antisense developed for the second conserved site of RT. The loop sequences were added from the hsa-mir-4660 and the flanking sequences were added from hsa-mir-2054.

FIG. 15 shows the sequence for the RT2, which has modifications to the consensus sequence to inactivate RT as well as the changes to the second conserved site against which the antisense miRNA was developed.

FIG. 16 contains the sequence for RT3, which has modifications designed to render the enzyme inactive as well as changes to both conserved sites to block binding by the designed miRNAs. Compared to the consensus sequence obtained for the RT, all the changes are within the 298th nt and the 723rd nt. The nucleotides that are underlined singly inactive the enzyme, those that are underlined doubly avoid self binding of antisense, and those that satisfy both conditions are underlined with wave pattern.

Design of Antisense miRNA Against Integrase.

### Active Site Modifications.

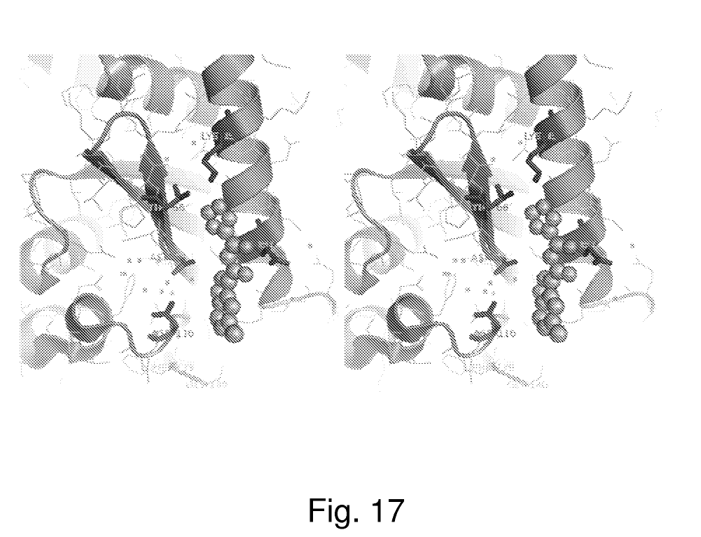

As described above for developing antisense sequences for RT, we have also created two antisense sequences against two sites for the enzyme integrase. For structural details, we have chosen 3nf6.pdb (Rhodes, et al, (2011)), 1ex4.pdb (Chen, et al, (2000)), and 1qs4.pdb (Goldgur, et al, (1999)) from the Protein Data Bank (Berman et, (2000)). Métifiot, et al, (2010) have discussed the antisense resistance from mutations in a loop region of integrase. As we are interested primarily to inactivate this enzyme, we will consider the integrase 3D structure around the catalytic residues. Robert Craigie and others (Jenkins. et al, (1997), Alian, et al, (2009)) have indicated that residues 64D, 116D, and 152E are key among those important for the catalysis. The active site of HIV-1 integrase is shown in FIG. 17, using the crystal structure 1qs4.pdb. The three residues are rendered as sticks, the inhibitor as small spheres. Apart from these residues, residues 66T and 159R (also shown in stick model) seem to be interacting with the inhibitor and represent good targets for mutations to inactivate the enzyme.

First, residue D64 is considered. From the alignment, the protein AF407656 has a glycine in that place. Thus, we have GAT to GGC. At 116D the protein AY314053 has a glycine. So, we could have GAC to GGT. Residue 152E has an arginine in protein GU216873 and we suggest GAA to CGT. Residue 66T has a valine, hence ACA to GTG. Residue 159R has an arginine in protein AF040274, hence AAG to CGT. These mutations will to make the enzyme inactive.

### Selecting the First Highly Conserved Integrase Site for Creating Antisense Sequences.

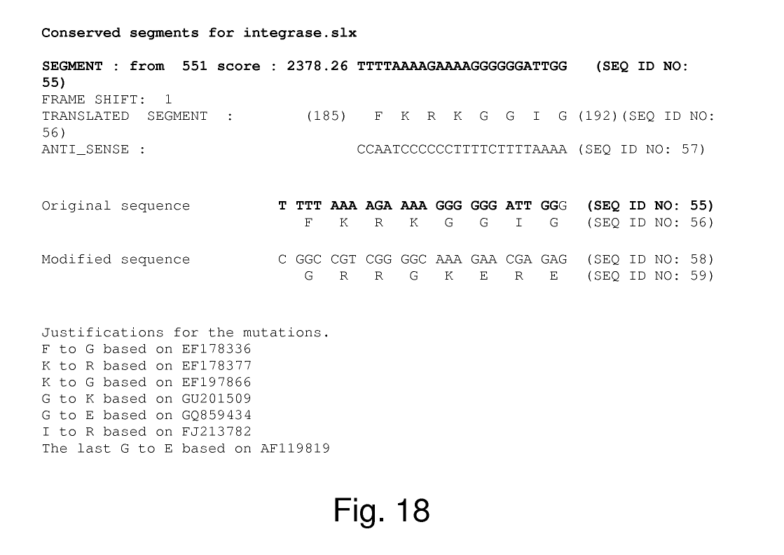

FIG. 18 details the first maximally conserved site within the integrase. The corresponding protein site seems to contain a loop in the protein and remains one of the more conserved sites. Mutations were suggested based on observations that were found in the sequences of other homologs or orthologs. Integrase is inactive as monomer (Gao, et al, (2001), Alian, et al, (2009)), and therefore this site should be important for activation through the formation of dimers/multimers. FIG. 18 contains the nucleotide sequence for the site and for the antisense. This segment (CCAATCCCCCCTTTTCTTTTAAAA (SEQ ID NO: 21)) contains 24 nucleotides, which could be considered as 3 sequences of 22 nucleotides. For one such sequence, (CCAATCCCCCCTTTTCTTTTAA (SEQ ID NO: 22)) we had three matches from the miRNA database for the 5′ side (CCAAT) and one match (TTAAA) for the 3′ end with another sequence (CAATCCCCCCTTTTCTTTTAAA (SEQ ID NO: 23)). We increased the design miRNA sequence length from 22 to 23 (CCAATCCCCCCTTTTCTTTTAAA (SEQ ID NO: 24)). Of the three matches for the 5′ end, two of the miRNAs MIMAT0004615 (hsa-mir-195) and MIMAT0004518 (hsa-mir-16-2) were found to be in the stem(down) orientation. We discarded them and used the last one, MIMAT0016905 (hsa-mir-4275). Similarly for the 3′ match, MIMAT0019950 (hsa-mir-1245b-5p) was found to be stem-up. Thus, we used the hsa-mir-1245b-5p for selecting the loop sequences and hsa-mir-4275 to design the flanking sequences. Also we have suggested mutations to the nucleotide sequence to avoid the binding of the antisense to the TV.

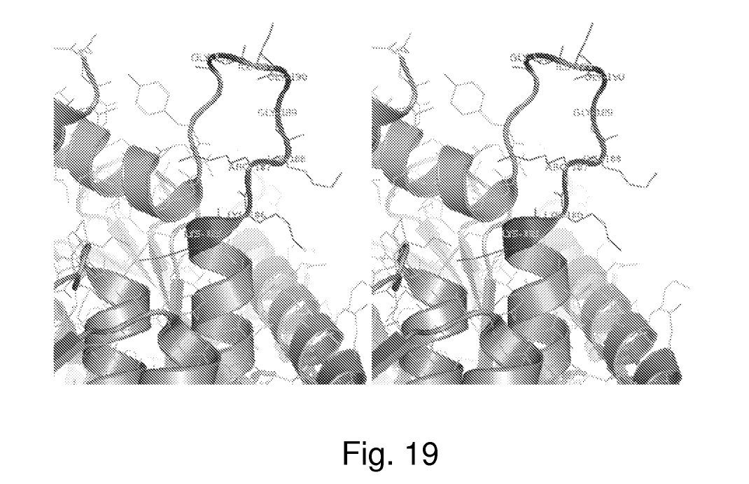

FIG. 19 details the location of this conserved site in the crystal structure 1qs4.pdb. Noteworthy, this conserved site is actually a loop connecting two helices accommodating a kink. One could engineer a proline to essentially produce the kink, and remove the loop. This strategy could eventually remove this site in the TV so that the designed antisense would have no place to bind.

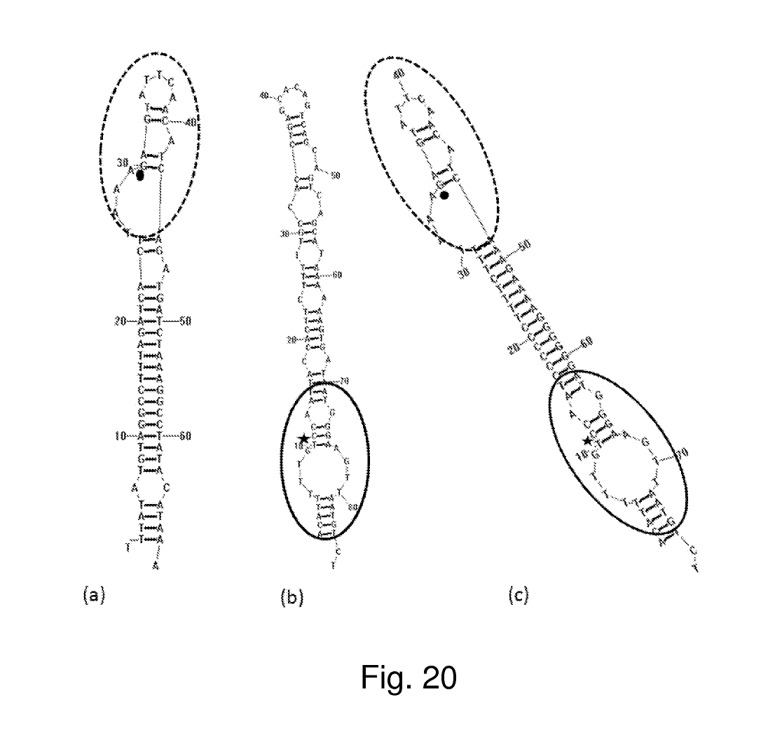

FIG. 20 shows the predicted secondary structures of (a) the hsa-mir-1245b-5p (for the loop design), (b) the hsa-mir-4275 (for the flanking sequences design), and (c) the shRNA of the antisense for the first conserved segment of integrase. This shRNA uses the loop from hsa-mir-1245b-5p and short flanking sequences from hsa-mir-4275 with the designed antisense as stem(up). In the picture, black dots denote the scissile bonds for the dicer, and the dark star denotes the scissile bonds for drosha.

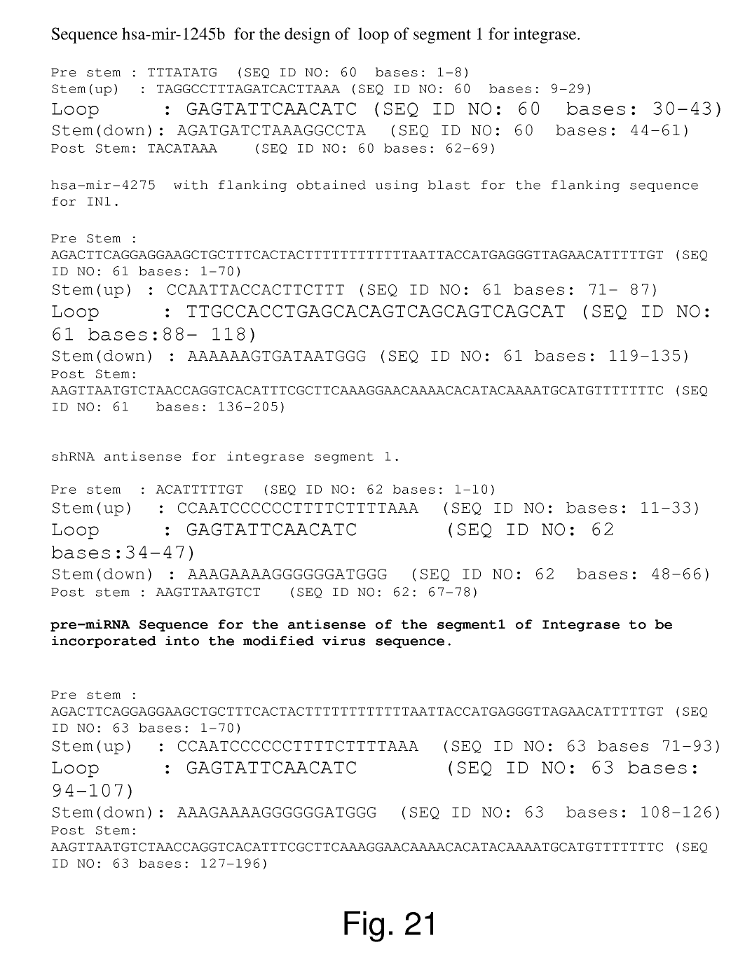

FIG. 21 shows the steps involved in the construction of the pre-miRNA antisense construct (stem(up)-loop-stem(down) along with the flanking sequences) derived by combining the proposed antisense for the first segment for the integrase with the loop sequences from the hsa-mir-1245b and flanking sequences from the hsa-mir-4275. Note that the last four nucleotide sequences have no complementary nucleotides in the stem-down sequence, a consequence of mimicking that part as in hsa-mir-1245b.

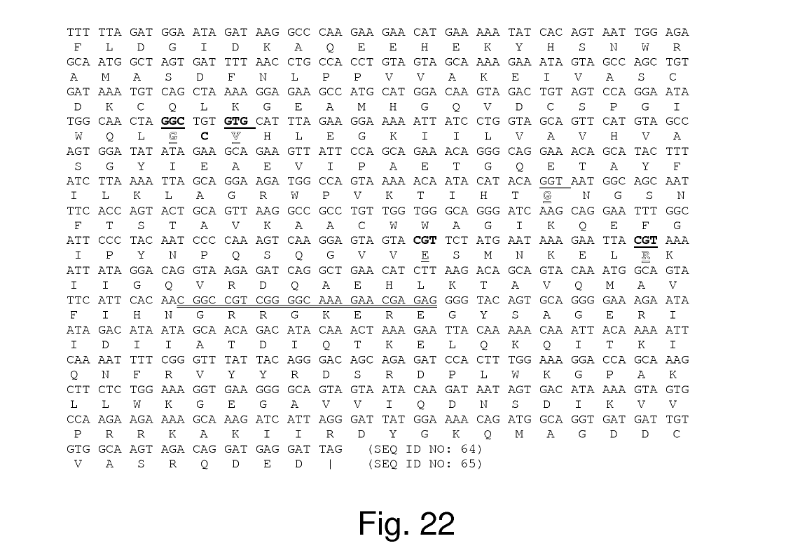

In FIG. 22, the genomic as well as protein sequences are given for integrase IN1 in the TV. The mutations to inactivate the integrase are shown as single underlines and the ones that are to protect the first conserved site from the miRNA developed against the corresponding segment within the infectious virus are underlined doubly.

### Selection of the Second Relatively Highly Conserved Integrase Site Antisense Target.

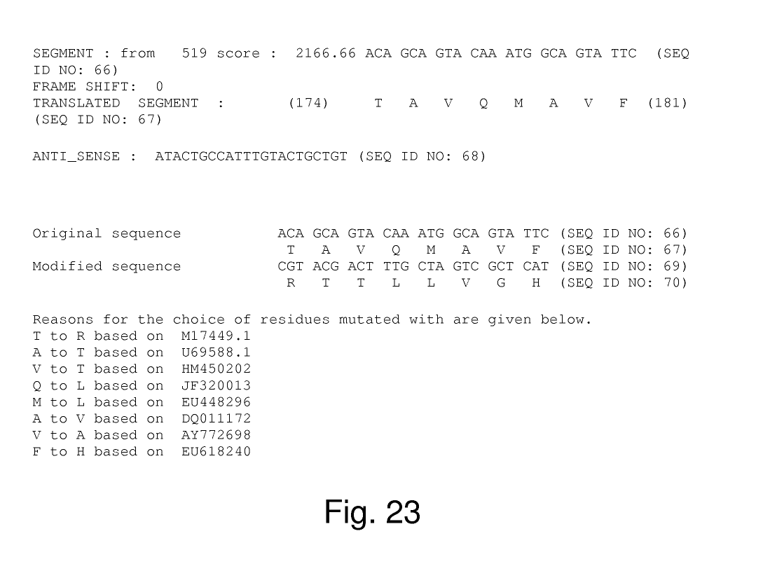

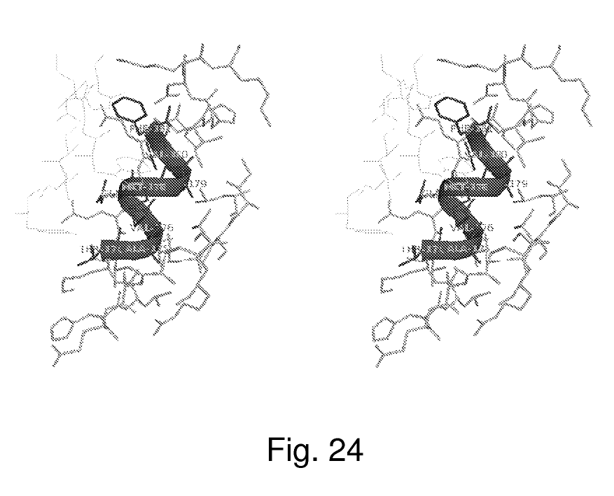

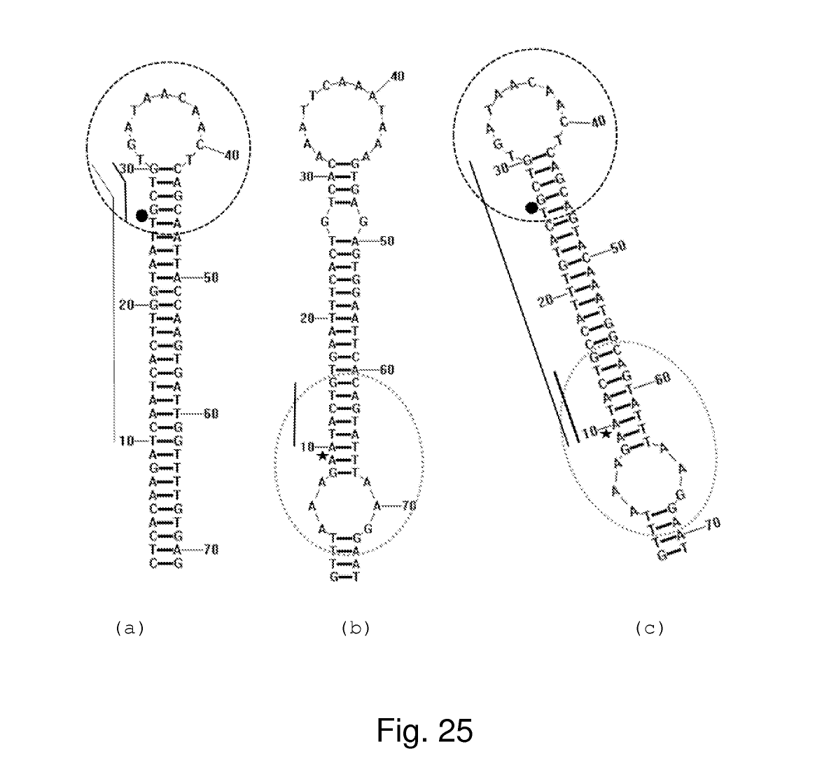

The next relatively highly conserved stretch happens to be adjacent to the first conserved site and is also at the dimer interface with a symmetry related molecule. FIG. 23 shows the second conserved site for integrase with the antisense, suggested mutations, and justifications provided separately. We use possible mutations to this site from observed mutations for those residues from the MSA with the provision that these changes do not disrupt the dimer formation as they do in nature. If the integrase exists as a dimer in the virus particle, the absence of dimer may disrupt the package formation.

FIG. 24 shows the second conserved site in the context of the active site using crystal structure 3nf6.pdb. The stretch (in ribbon) and the residues from a symmetry-related molecule forming the interface are shown in light gray.

FIG. 25 shows the predicted secondary structure of selected shRNAs (a) hsa-mir-4705 for integrase segment 2 loop sequences, (b) hsa-mir-4693-5p for flanking sequences, and (c) for the shRNA of the designed miRNA. The parts that are maintained for the loop design and the flanking residues design are enclosed in dotted circles. The scissile bonds for drosha and dicer are marked by a black dot and a black star, respectively. Antisense for second segment for integrase is ATACTGCCATTTGTACTGCTGT (SEQ ID NO: 25).

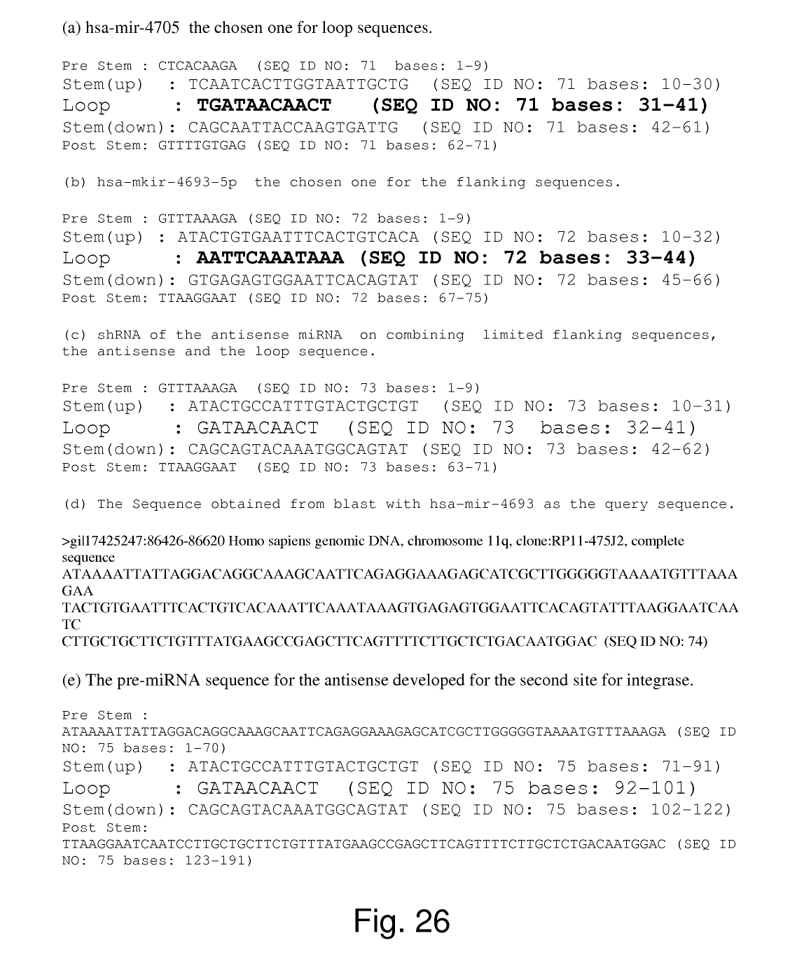

FIG. 26 shows the steps involved in arriving at the pre-miRNA construct (stem-loop-stem with flanking sequences) for the second relatively highly conserved site within the genomic sequence of integrase, derived by combining the designed antisense sequence, and the flanking sequences obtained from the transcript of hsa-mir-4693-5p using “blastn” against the human nucleotide database and the loop sequence from hsa-mir-4705.

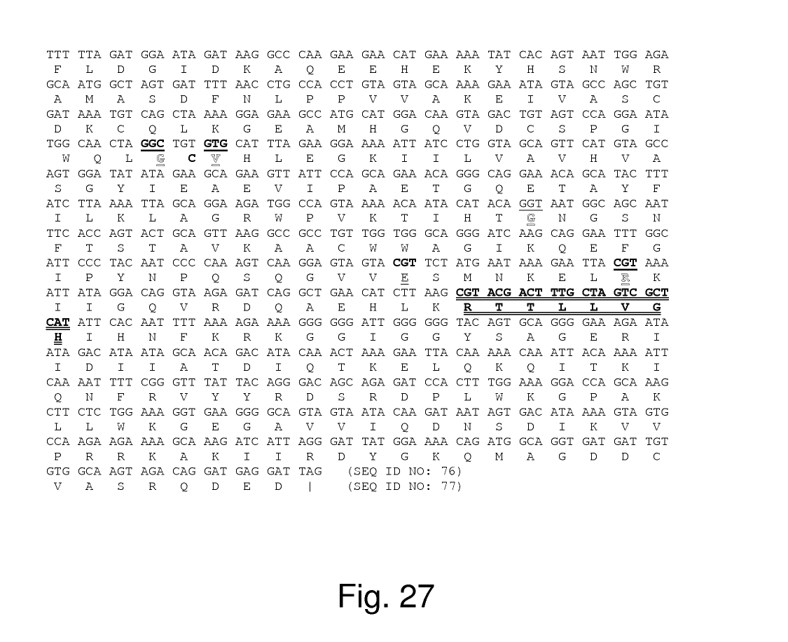

FIG. 27 contains the consensus sequence for integrase (IN2) with modifications to inactivate the enzyme underlined singly as well as modifications made to the conserved sites to avoid self binding of the antisense sequences developed against the second conserved site, underlined doubly.

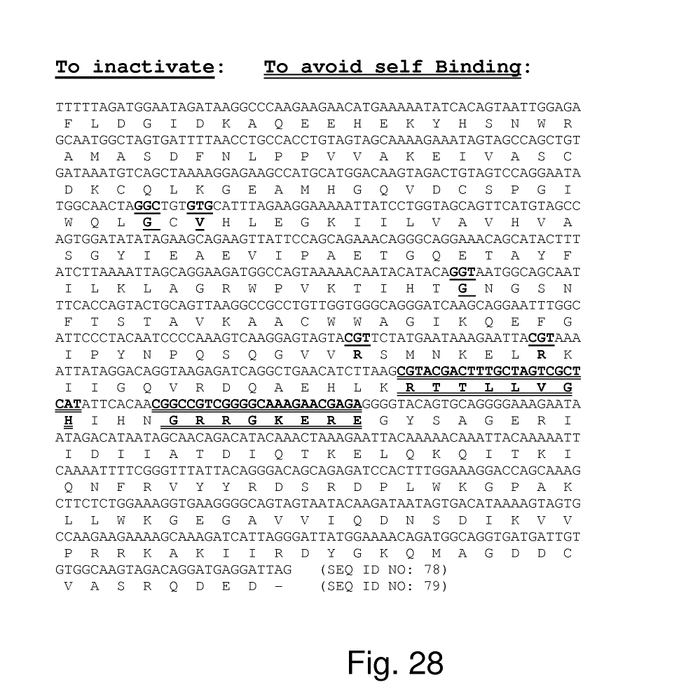

FIG. 28 shows the consensus sequence for integrase IN3 with modifications to inactivate the enzyme as well as modifications made to both conserved sites to avoid self binding of antisense sequences.

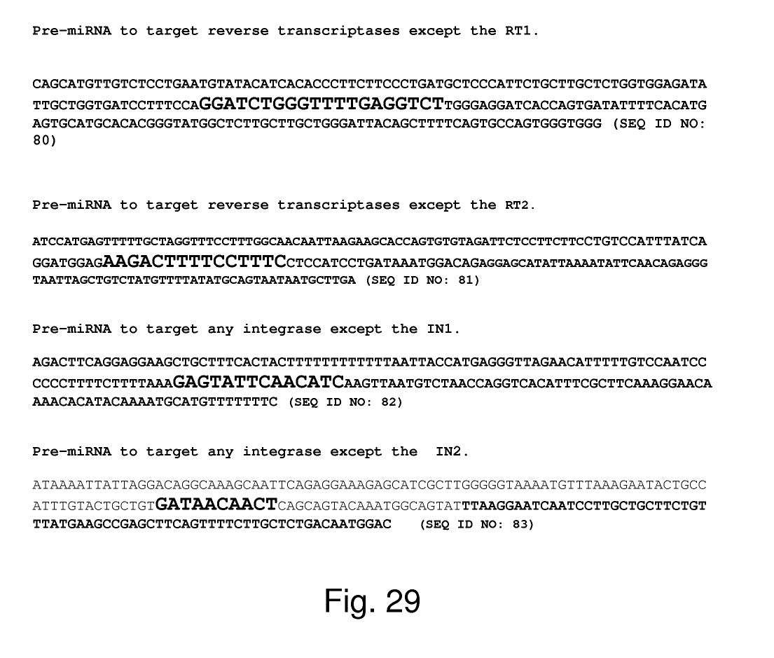

FIG. 29 contains the constructs for the antisense sequences, two for the reverse transcriptase and two for the integrase, to be inserted into the genomic sequence for the TV.

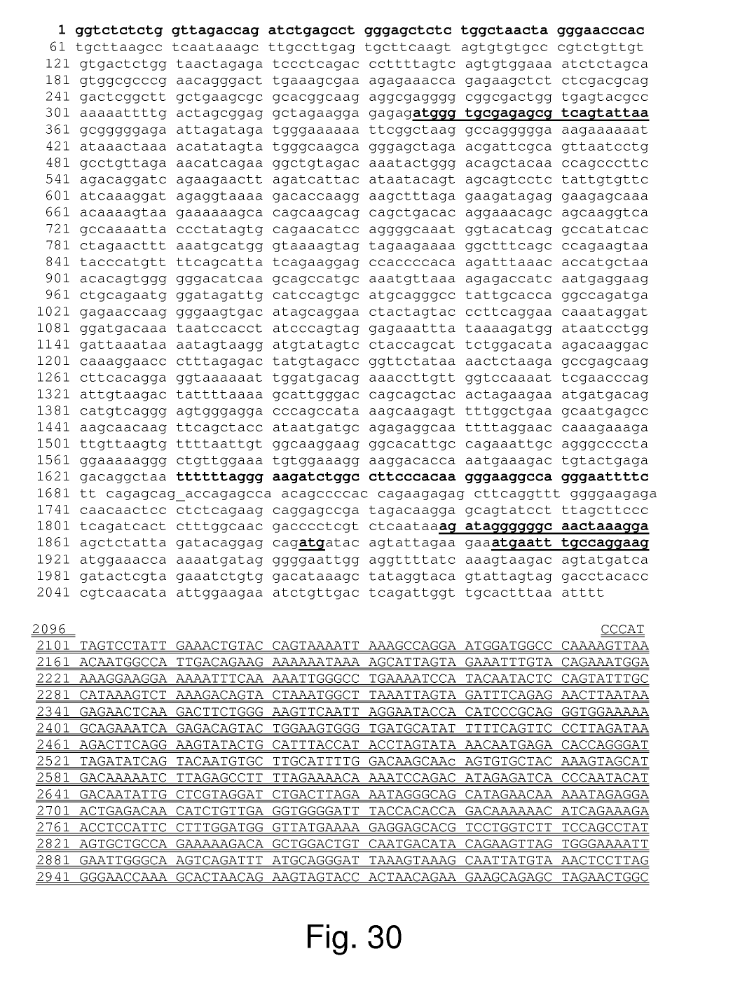

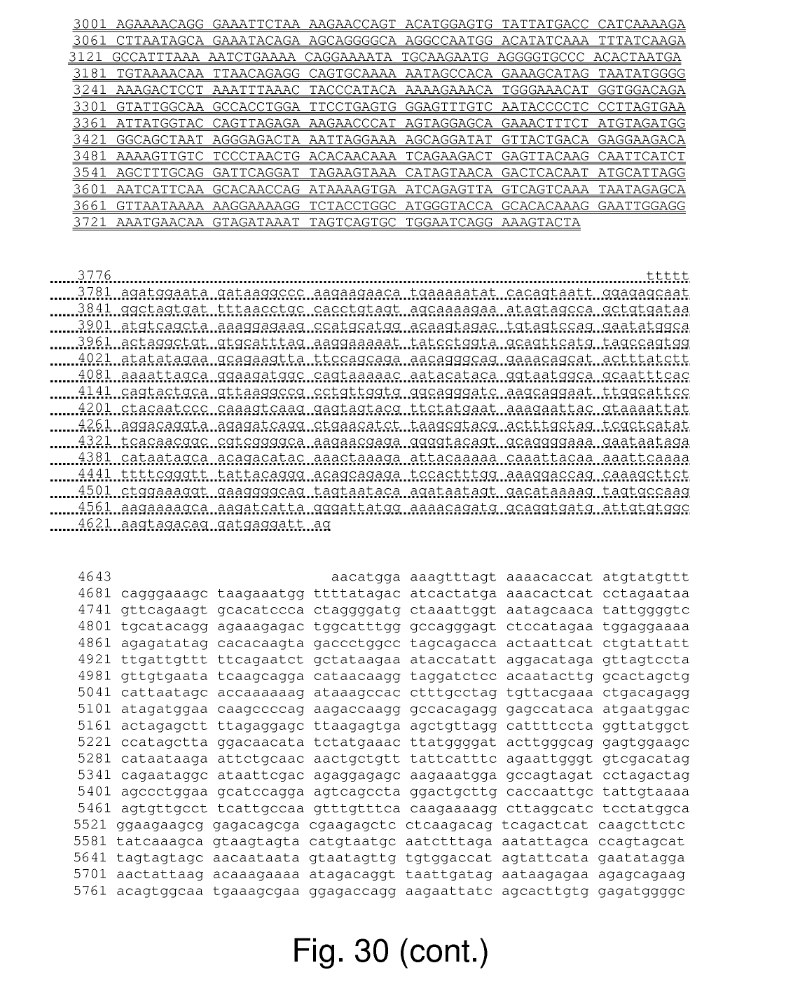

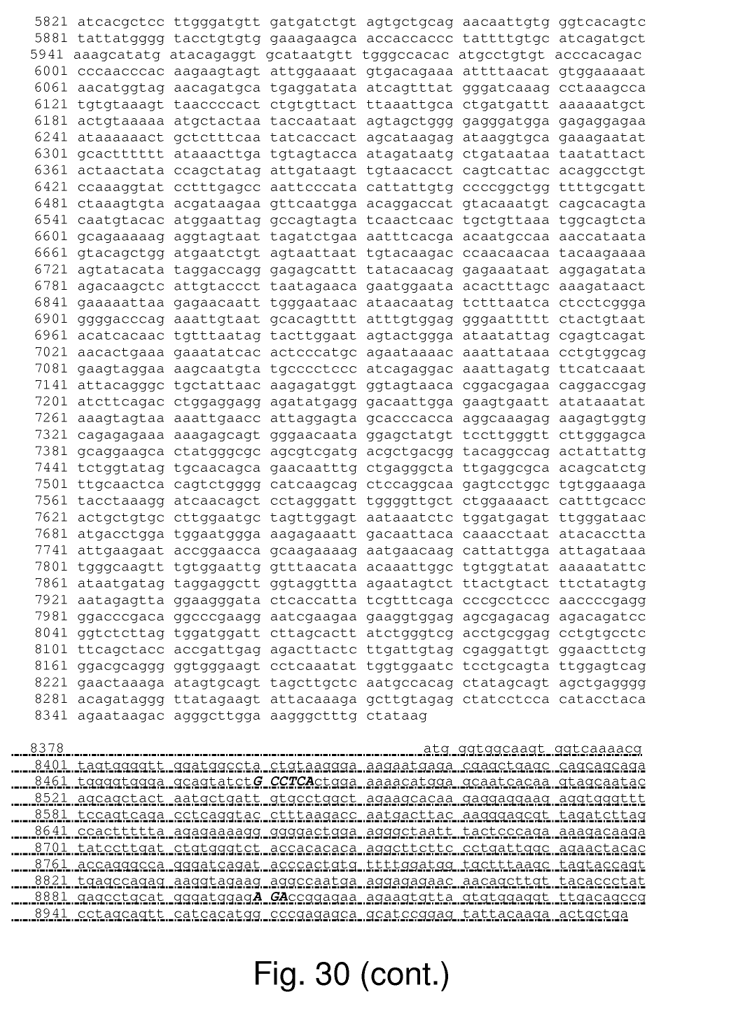

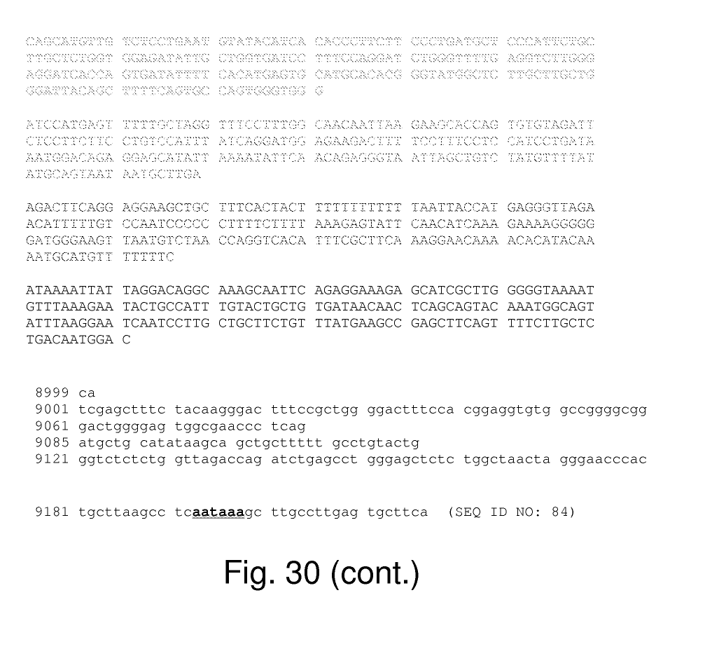

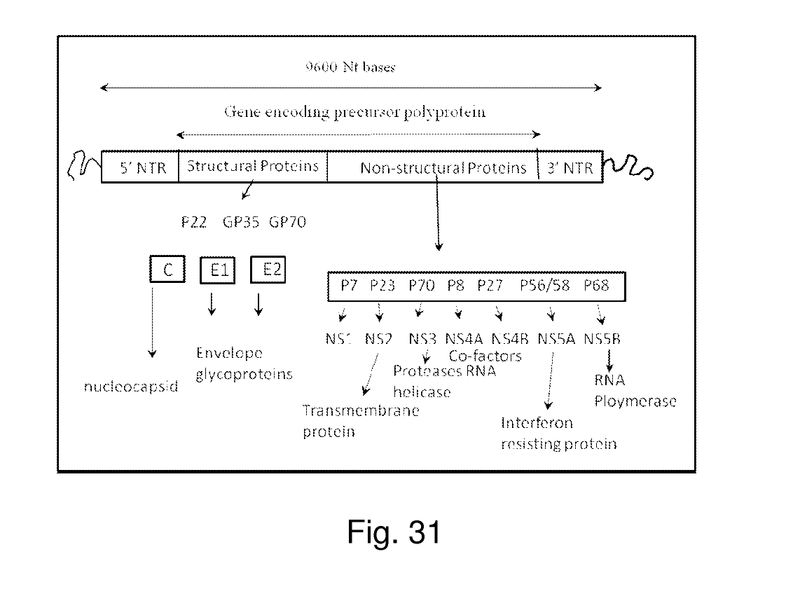

FIG. 30 contains the genomic sequence for the attenuated TV. This sequence has been numbered sequentially, though we have separated some of the genes of interest by breaks and underlining them. The RT domain is marked by double underlines, the integrase domain with dotted underlines, and the Nef domain is underlined with dots/dashes. The RT domain has the modifications expected to inactivate the enzyme and those to avoid binding by the antisense. Similarly, the integrase domain also contains the required modifications to inactivate the integrase and to avoid binding of the antisense (designed miRNAs) created against the integrase sites. The mutations to inactivate the enzymes are meant to attenuate the TV. The antisense sequences are to be inserted after the stop codon of the Nef domain, so that the sequences meant to be antisense will be in the untranslated region of the TV. The antisense sequences are to be introduced into the genome of the TV well before the 3′ end LTR sequence and the LTR sequence, underlined with double-waves in FIG. 30.

Several articles (Garcia, et al, (1993), Aiken, et al, (1994), Bentham, et al, (2003), and Hanna, et al, (2006)) implicate the Nef domain of HIV-1 to the down regulation of CD4 cell expression. Hanna, et al, (2006) identified that mutation in the Nef domain RD35/36AA and D174K abrogated the down regulation of the CD4. Bentham, et al, (2003) reported that di-leucines at 413/414 of CD4 are essential for the down regulation. Further, the di-leucines are needed for endocytosis and not for binding to the Nef domain of HIV-1. We do not want our TV, to down regulate CD4 cells and have tried to incorporate this goal into our design model. However the mutations were slightly modified to have maximum dissimilarity of the original triplets for these residues so that the probability of going back to the original sequences is low. We have changed the residue R35, which has the codons “cga” to alanine (gcc); D36S (gac to tca) and D174R (gac to aga). The modified codons for these residues are shown in italics in the FIG. 30.

### Possible Constructs for the Therapeutic Virus.

The total length of the RNA in the infectious virus is 9217:

Sequence extending from 1 to 2100 are designated the A section.

Sequence extending from 2001 to 4650, or the RT part, are designated the B section.

Sequence extending from 4651 to 8400 are designated the C section.

Sequence extending from 8401 to 9000, or the integrase part, are the D section.

The insertion of pre-miRNAs after 9000 is designated the E section.

Sequence extending from 9001 to 9217 is designated the F section.

The B section has variations at two sites when compared to wild type. One (B1) contains mutations to first site against which antisense was created referred to as RT1 in the text. The other one (B2) contains mutations to second site against which antisense was created referred to as RT2 in the text. We can consider one (B3) that contains mutations to both first and second sites and is called as RT3 within the text. The complete absence of RT could be called (B4).

The wild type integrase would be (D0). The section D has variations at two sites when compared to wild type. One (D1) contains mutations to first site against which antisense was created referred to as IN1 in the text. Another one (D2) contains mutations to second site against which antisense was created referred to as IN2 in the text. We can consider one (D3) that contains mutations to both first and second sites and is called referred to as RT3 in the text. The complete absence of integrase could be called (D4).

There are 4 pre-miRNAs two for RT and two for integrase. These are individually called E1, E2, E3, and E4.

One can in principle make all possible and meaningful combinations. The one that includes all the four pre-miRNAs would be preferable as this should be more effective and less prone to spontaneous mutations that make it ineffective. At the same time, when we include 4 pre-miRNAs, the volume of the matter within the capsid is increased and packaging may be less efficient. Optionally, we should be looking at the ones which have lesser pre-miRNAs.

The following are the designs for various therapeutic virus. Therapeutic virus genome comprise, e.g., A+B+C+D+E+F, where B could have 4 possibilities, D could have 4 possibilities, and E could have E1, E2, E3, and E4 individual pre-miRNA. We can also have constructs without any modifications to integrase (wild type) and in this case no E3 or E4 will be used as the presence of E3 and E4 would be targeting the TV and destroy them unless the TV does not contain the integrase at all. The one that contains mutations to both miRNA binding sites of both RT and integrase and including all the four miRNAs is given below:
A+B3+C+D3+E1+E2+E3+E4+F

One can optimize the TV by going through combinations of the B's, D's and E's by constructing many possibilities to verify which are most efficient at producing virus particles and selecting the best. Importance is given to maximum number of pre-miRNAs included in the TV and also producing virus particles. The following list contains a few combinations where at least one of the miRNA binding sites on the reverse transcriptase is included. It is envisioned, and well within the skill in the art, for one to create a similar listing where at least one site for the integrase is maintained, based on the teachings herein.
A+B3+C+D3+E1+E2+E3+E4+F
A+B1+C+D3+E1+E3+E4+F
A+B2+C+D3+E2+E3+E4+F
A+B3+C+D1+E1+E2+E3+F
A+B3+C+D2+E1+E2+E4+F
A+B3+C+D0+E1+E2+F
A+B1+C+D1+E1+E3+F
A+B1+C+D2+E1+E4+F
A+B2+C+D1+E2+E3+F
A+B2+C+D2+E2+E4+F
A+B1+C+D0+E1+F
A+B2+C+D0+E2+F

Based on the teachings above, one may prepare any number of functional therapeutic viruses against HIV. Further, using the general concepts provided, one may design any number of additional TV constructs against any number of different viruses. For example, see HCV construct, below.

### Example 2

### Design of Therapeutic Virus Against Hepatitis C Virus

As a second example, we have chosen to design a therapeutic virus against hepatitis C virus (HCV). HCV is a positive-sense single stranded RNA virus. A description of the same can be found in http://en.wikipedia.org/wiki/Hepatitis_C_virus. The genome organization of the Hepatitis C virus is given in FIG. 31 as presented at the wikipedia site. The virus encodes structural proteins and non-structural proteins flanked on either sides by NTRs (non-translated regions). One open reading frame encodes a polyprotein of 3010 amino acids. This protein is cut by viral and cell enzymes to active proteins. One of the enzymes of the non-structural proteins is the NS5B which is a RNA dependent RNA polymerase (RDRP). This enzyme is useful at the initial stages in the propagation of the virus. Hence, we would intervene in the translation process of this enzyme using miRNA (micro RNA). Our therapeutic virus will be devoid of this enzyme or would have a modified as well as inactive enzyme. We expect this enzyme from the infectious virus to perform its function on the therapeutic virus. Once it is done, the miRNA from the therapeutic virus will interfere with the translation process of this enzyme, thus stopping the progress of the infectious virus. At the same time, this also ensures that the therapeutic virus will work only in the cells which are already infected with HCV. We have designed a pre-miRNA for introduction into the therapeutic virus, before the 3′ end NTRs.

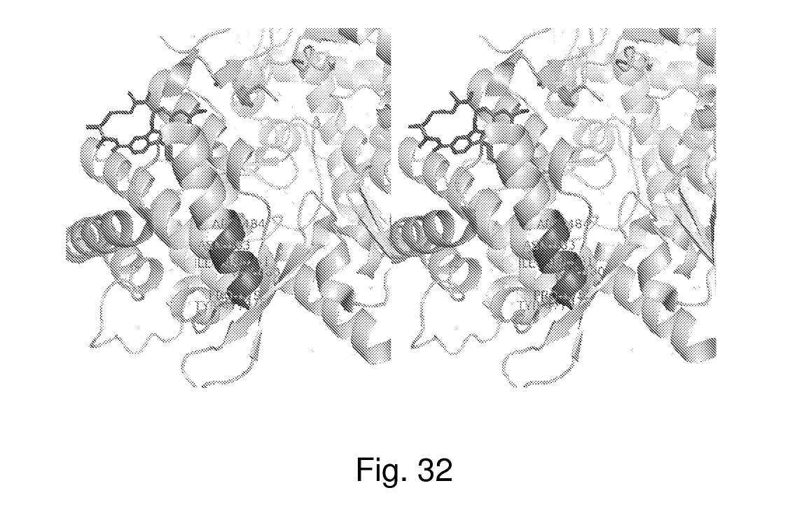

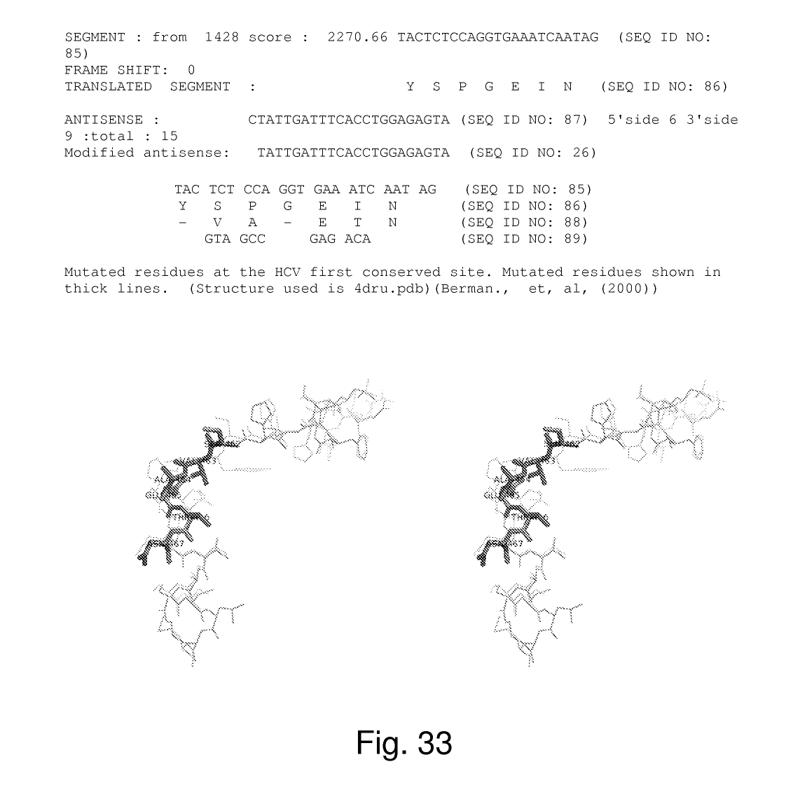

Extract the nucleic acid sequences for the NS5B enzyme a RDRP and form one of the nucleotide sequences for the complete genome of the HCV virus from the NCBI database http://www.ncbi.nlm.nih.gov/ with the accession number AB520610 (Weng et al., (2010), Arai et al., (2009)). We used this sequence to do a “nucleotide blast” run against the nucleotide database with options as described earlier. We used the alignments of 1053 sequences, as given in the “blast” output to find out highly conserved stretches for a window of 22 nucleotide long. The best relatively conserved stretch of nucleotides is shown in FIG. 32 and the details are given in FIG. 33. The actual sequence is “TATTGATTT CACCTGGAGAGTA (SEQ ID NO: 26)”. The matured miRNA that matched the 3′ end of the sequence is MIMAT0003386, which is in the hairpin sequence (shRNA) hsa-mir-376a.

The matching elements being “GAGTA”. The MIMAT0003386 happens to be in the stem_up orientation within the shRNA. Similarly, the matured miRNA that matched the 5′ end of the antisense is MIMAT0000090 and the shRNA is hsa-mir-32. The matching sequence is “TATTG”.

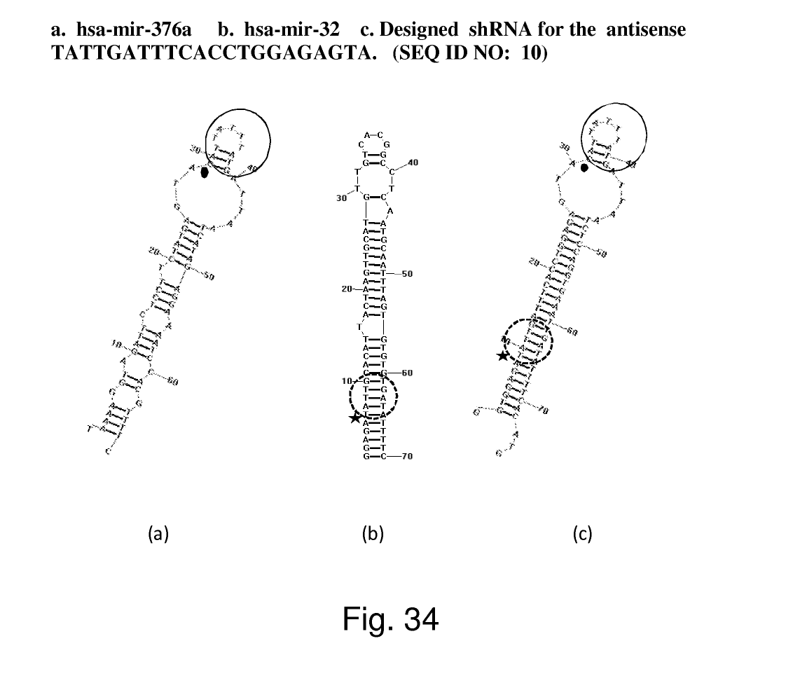

The predicted secondary structure of hsa-mir-376a is given in FIG. 34a. From this, we get the loop sequences as well as the sequence that is complementary to the matched sequences. Similarly, the predicted secondary structure for the hsa-mir-32 is given in FIG. 34b. From this, we get the sequences complementary to the matched sequences at the 5′ end. Then, the hsa-mir-32 was used as a query in a “blast” run, and its primary sequence was obtained and from this, we obtained the flanking sequences of length >60 nts.

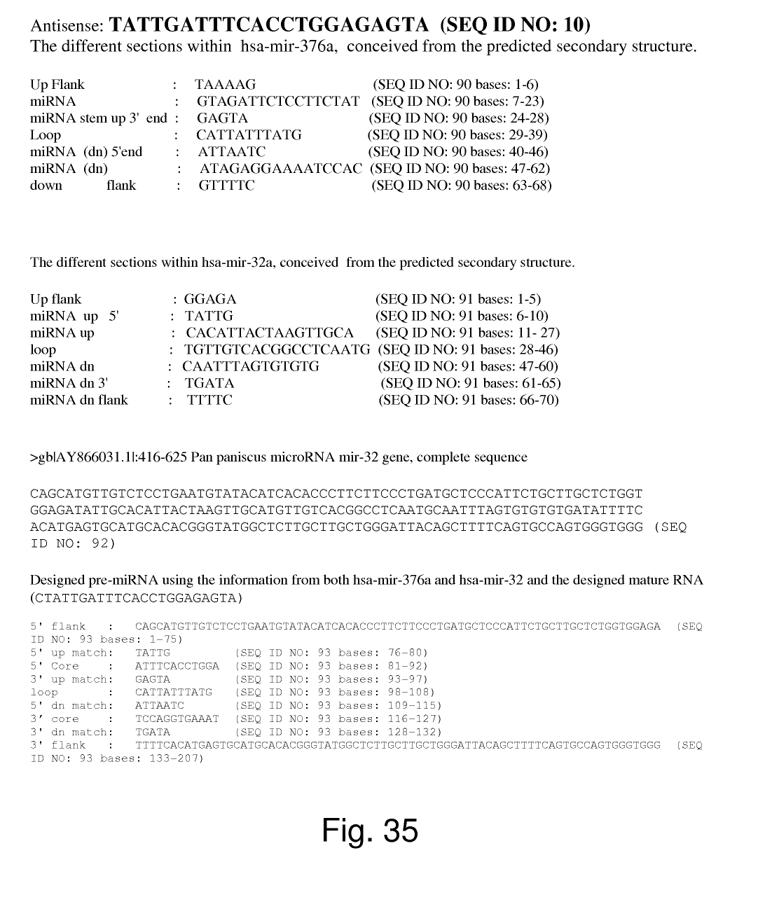

The final pre-miRNA for the antisense was designed by combining various segments described above which is given in FIG. 35.

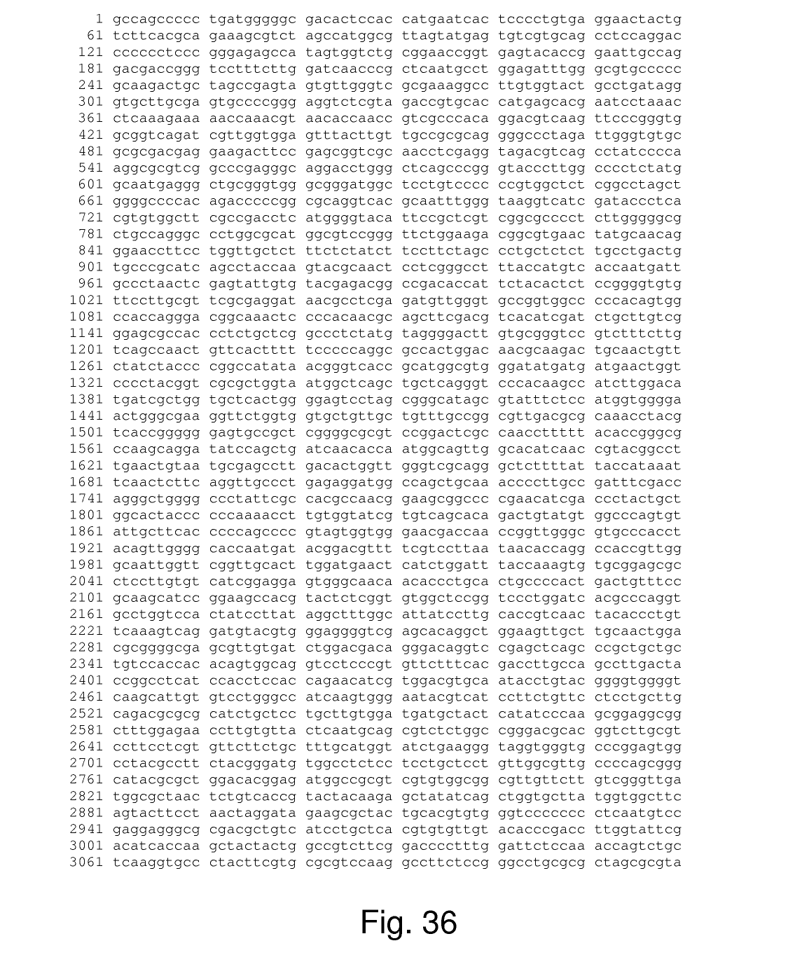

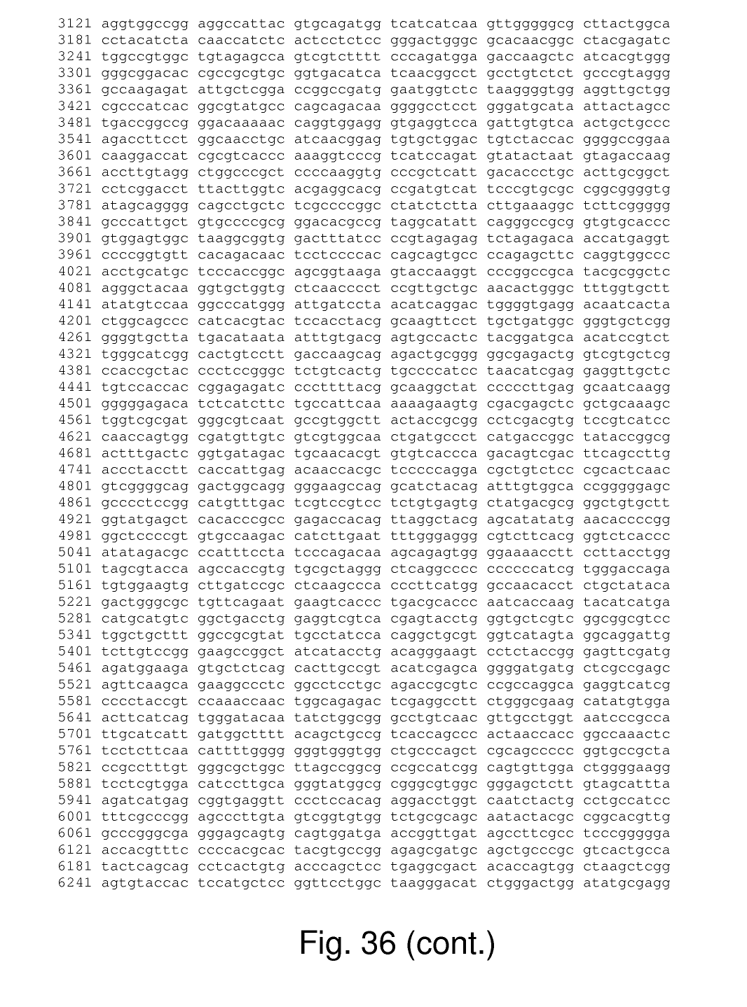

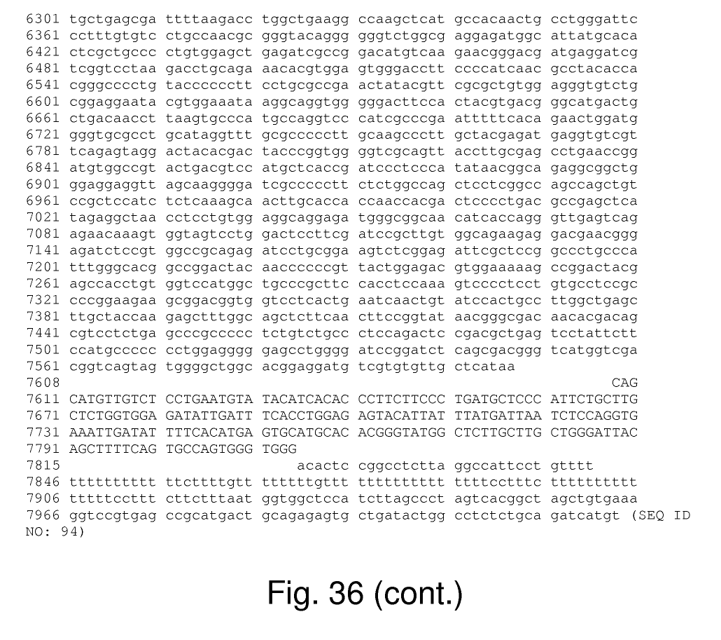

In FIG. 36, we give the sequence for the therapeutic virus against HCV. Compared to the infectious virus genome, this one lacks the coding for the NS5B gene and a stop codon was introduced after the NS5A gene. After the stop codon, we have introduced the pre-miRNA containing the antisense, and then the 3′ NTRs were left undisturbed.

It is expected that such a therapeutic virus administered, should enter the hepatocytes of the liver as the structural proteins are still kept intact. Once inside the cell, as this lacks the RDRP enzyme, it will need the RDRP produced by the infectious virus (if the cell has already been infected with HCV) to get through the initial stages of infection. During the progression of cell cycle, miRNA will be produced by host enzymes “drosha” and “dicers” from the pre-miRNA introduced in the NTR region. The miRNA would enter the RISC (RNA-induced silencing complex) complex, to interfere with the translation of the RDRP and the production of infectious virus would be stopped. During this time, the therapeutic virus will continue to be produced and released to enter other hepatocytes and this process should continue until the infectious virus completely destroyed.

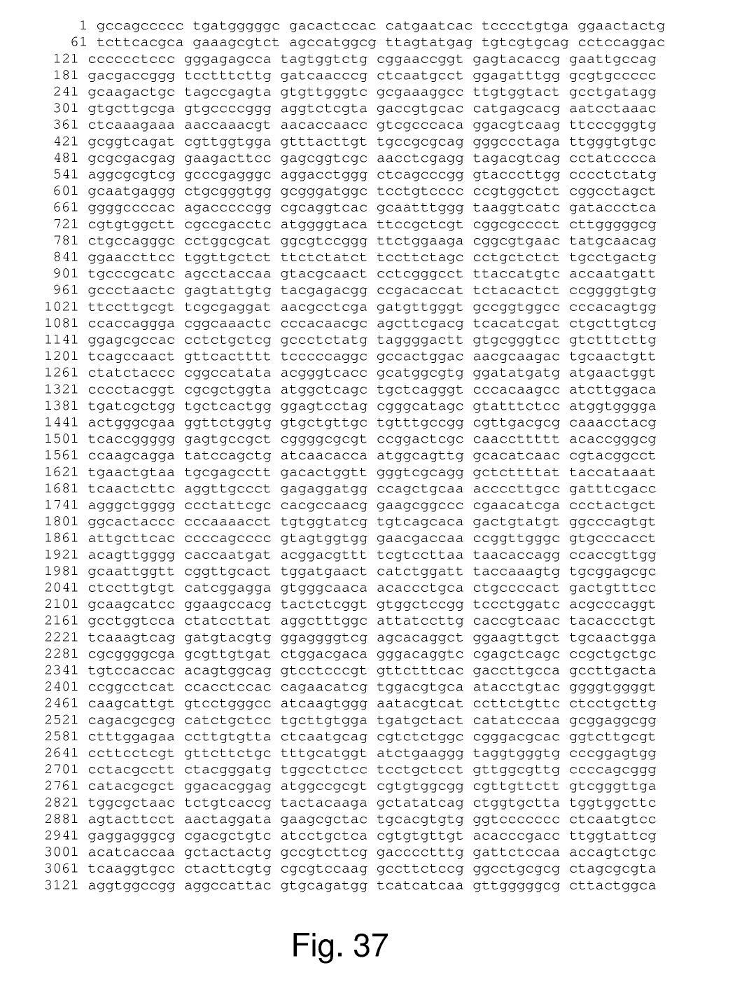

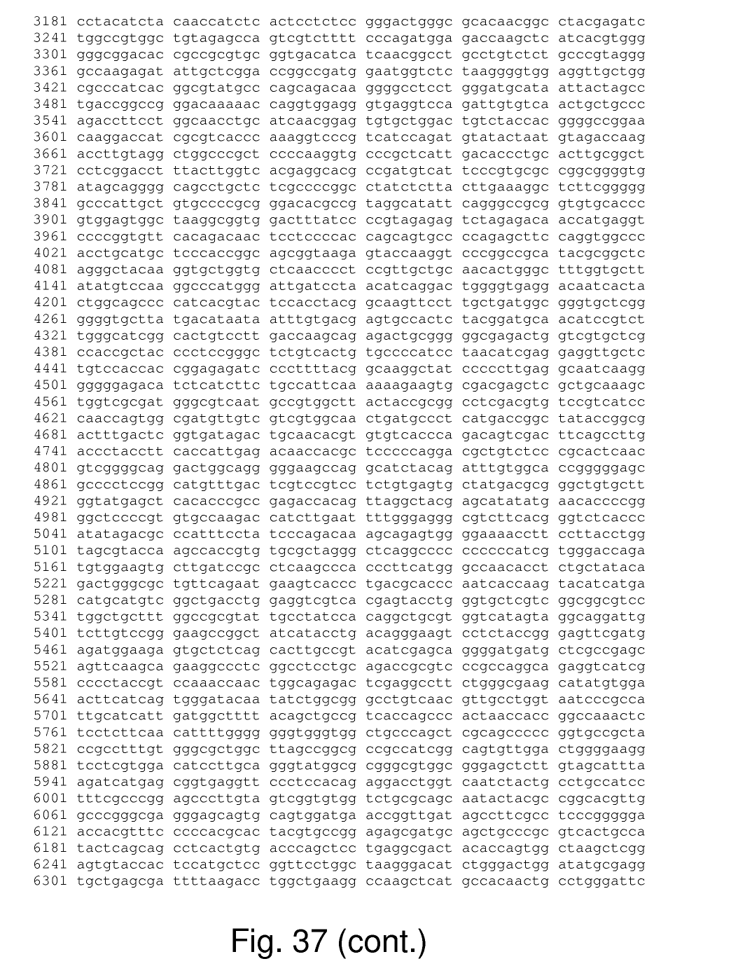

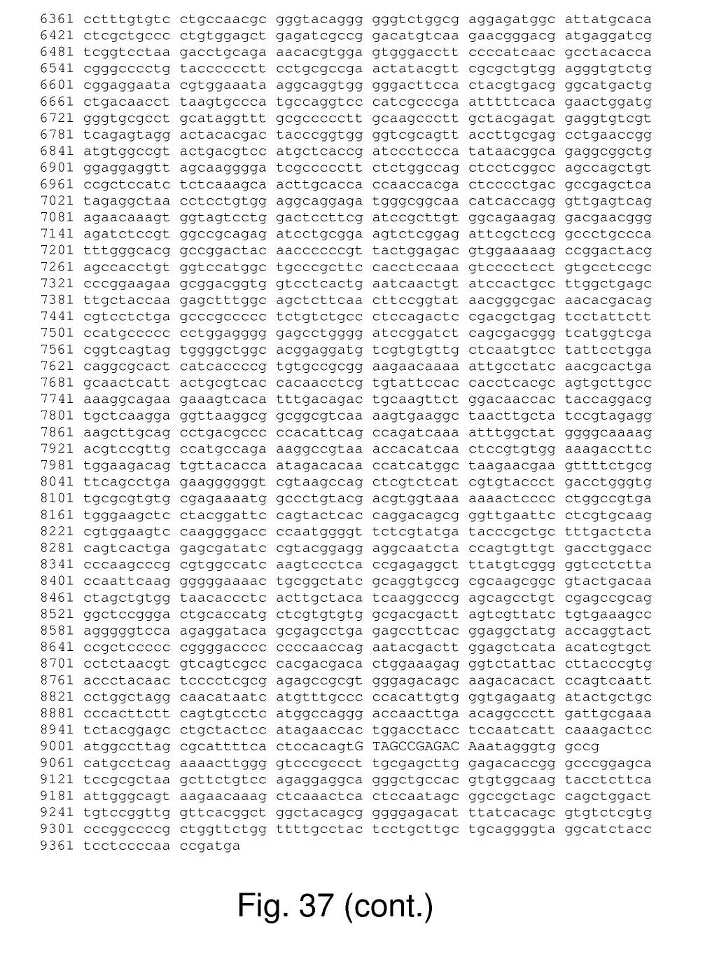

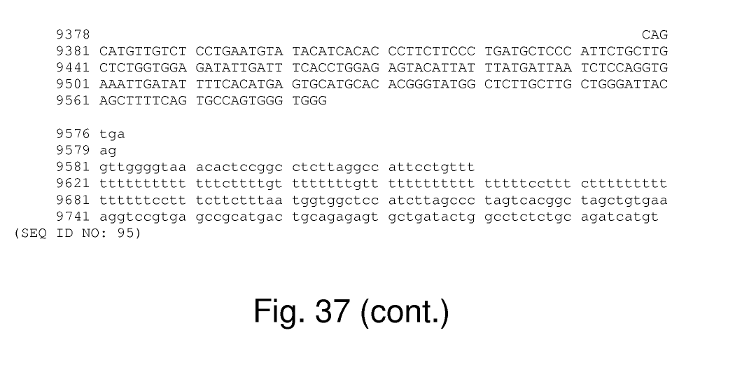

We give here genome sequences for two versions for the therapeutic virus. In FIG. 36, we have a version where the coding for the gene NS5B-1 is completely removed and the pre-miRNA sequences that would interfere with synthesis of the NS5B-1 protein of the infectious virus included before the 3′ NTR. The second version is given in FIG. 37, though containing the coding for the gene NS5B-1, the conserved site against which the miRNA was designed, has been modified so that the miRNA would not bind and disrupt the production of the mutated NS5B-1 gene. This is version is designed to better retain the three dimensional packing of the proteins by avoiding the deletion of the NS5B-1 gene from its genome. The program MODELLER (Marti-Renom et al., (2000), Sali & Blundell (1993), Fiser et al., (2000)) was used to model the mutations and deletions in the crystal structure 4dru.pdb (Cummings et al., 2012).

### Example 3

### miRNA Designs

A. A pre-miRNA is adapted to have cleavage sites for efficient and precise processing by enzymes dicer and drosha within the host cells to provide miRNA within an L-sshRNA or R-sshRNA. The pre-miRNA can include the following.

A sense sequence miRNA, a loop sequence at the 3′ end of the sense sequence, an antisense to sense miRNA at the 3′ end of the loop sequence, and 5′ flanking (F5) at the 5′ end of the sense sequence and 3′ flanking (F3) sequences at the 3′ end of the antisense sequence; at least 6 nucleotides, at least three from the 3′ end of sense miRNA and at least three from the 5′ end of the loop sequence and 6 nucleotides, at least three from 3′end of the loop sequence and at least three from the 5′ end of the antisense matches a similar construct from any shRNA from any miRNA database available at that time; at least 6 nucleotides, that is at least three from the 5′ end of the sense miRNA and at least three from the 3′ end of F5 and at least 6 nucleotides, that is at least three from the 3′ end of the antisense sequence and at least three from the 5′ end of the F3 matches a similar construct from any pre-miRNA from any miRNA database available at that time; the F5 has at least 95% sequence identity and F3 has at least 95% sequence identity to the corresponding flanking sequences of the same pre-miRNA from the miRNA database; and, a sense sequence miRNA, a loop sequence at the 5′ end of the sense sequence, an antisense to sense miRNA at the 5′ end of the loop sequence and 5′ flanking (F5) at the 5′ end of the antisense sequence and 3′ flanking (F3) sequences at the 3′ end of the sense sequence; at least 6 nucleotides, at least three from the 3′ end of the loop sequence and at least three from the 5′ end of sense miRNA and 6 nucleotides, at least three from the 3′ end of the antisense and at least three from the 5′ end of the loop sequence matches a similar construct from any shRNA from any miRNA database available at that time; at least 6 nucleotides, that is at least three from the 3′ end of the sense miRNA and at least three from the 5′ end of F3 and at least 6 nucleotides, that is at least three from the 3′ end of the F5 and at least three from the 5′ end of the antisense sequence matches a similar construct from any pre-miRNA from any miRNA database available at that time; the F5 has at least 95% sequence identity and F3 has at least 95% sequence identity to the corresponding flanking sequences of the same pre-miRNA from the miRNA database.

B. A highly conserved stretch of given length within a gene can include the following.

A high scoring stretch of given length, which is the sum of the scores of each position of the stretch; where the score is the frequency of occurrence of a particular nucleotide at the given position or column which is the maximum compared to frequency of occurrence of other possible nucleotides occurring at that position or column, calculated from a multiple sequence alignment of sequences homologous (at least 80% identity) to the gene; which is used to design antisense or miRNA that would hybridize with the conserved stretch.

C. A designed L-sshRNA (stem(up) shRNA) can include the following.

An miRNA (sense sequence) at the 5′ end of the shRNA; a loop sequence at the 3′ end of the sense sequence and a sequence (antisense) complimentary to the miRNA or the sense sequence at the 3′ end of the loop sequence; at least three nucleotides from the 3′ end of the miRNA, the whole loop sequence, and at least three sequences at the 5′ end of the antisense sequence match an shRNA from any miRNA database; whereby the shRNA is adapted to have a natural cleaving sites for dicer.

D. A designed R-sshRNA (stem(down) shRNA) can include the following.

An miRNA (sense sequence) at the 3′ end of the shRNA; a loop sequence at the 5′ end of the sense sequence and a sequence (antisense) complimentary to the miRNA or the sense sequence at the 5′ end of the loop sequence; at least three nucleotides from the 5′ end of the miRNA, the whole loop sequence, and at least three sequences at the 3′ end of the antisense sequence match an shRNA from any miRNA database; whereby the shRNA is adapted to have a natural cleaving sites for dicer.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

### APPENDIX

### Cited References

### References.

Adachi A, Gendelman H E, Koenig S, Folks T, Willey R, Rabson A, Martin M A. Production of acquired immunodeficiency syndrome-associated retrovirus in human and nonhuman cells transfected with an infectious molecular clone. J. Virol. 1986 August; 59(2):284-91.
Aiken C, Konner J, Landau N R, Lenburg M E, Trono D. Nef induces CD4 endocytosis: requirement for a critical dileucine motif in the membrane-proximal CD4 cytoplasmic domain. Cell. 1994 Mar. 11; 76(5):853-64.
Aldovini A, Young R A. Mutations of RNA and protein sequences involved in human immunodeficiency virus type 1 packaging result in production of noninfectious virus. J. Virol. 1990 May; 64(5):1920-6.
Alian A, Griner S L, Chiang V, Tsiang M, Jones G, Birkus G, Geleziunas R, Leavitt A D, Stroud R M. Catalytically-active complex of HIV-1 integrase with a viral DNA substrate binds anti-integrase drugs. Proc Natl Acad Sci USA. 2009 May 19; 106(20):8192-7. Epub 2009 May 4.
Altschul S F, Madden T L, SchÃ [symbol] ffer A A, Zhang J, Zhang Z, Miller W, Lipman D J. Gapped BLAST and PSI-BLAST: a new generation of protein database search programs. Nucleic Acids Res. 1997 Sep. 1; 25(17):3389-402.
Ambros V, Bartel B, Bartel D P, Burge C B, Carrington J C, Chen X, Dreyfuss G, Eddy S R, Griffiths-Jones S, Marshall M, Matzke M, Ruvkun G, Tuschl T. A uniform system for microRNA annotation. RNA. 2003 March; 9(3):277-9.
Beckett R, Miller W A. Rapid full-length cloning of nonpolyadenylated RNA virus genomes. Curr Protoc Microbiol. 2007 February; Chapter 16:Unit 16F.3.
Benson, D. A., Karsch-Mizrachi, I., Lipman, D. J., Ostell, J. and Wheeler, D. L. GenBank. Nucleic Acids Res. 2003, 31, 23-27.
Bentham M, Mazaleyrat S, Harris M. The di-leucine motif in the cytoplasmic tail of CD4 is not required for binding to human immunodeficiency virus type 1 Nef, but is critical for CD4 down-modulation. J Gen Virol. 2003 October; 84(Pt 10):2705-13.
Berman H. M, Westbrook J, Feng Z, Gilliland G, Bhat T. N, Weissig H, Shindyalov I. N, Bourne P. E. The Protein Data Bank. Nucleic Acids Research, 2000 28:235-242.
Biswas P, Jiang X, Pacchia A L, Dougherty J P, Peltz S W. The human immunodeficiency virus type 1 ribosomal frameshifting site is an invariant sequence determinant and an important target for antiviral therapy. J Virol. 2004 February; 78(4):2082-7.
Bocharov G, Ford N J, Edwards J, Breinig T, Wain-Hobson S, Meyerhans A. A genetic-algorithm approach to simulating human immunodeficiency virus evolution reveals the strong impact of multiply infected cells and recombination. J Gen Virol. 2005 November; 86(Pt 11):3109-18.
Boden D, Pusch O, Silbermann R, Lee F, Tucker L, Ramratnam B. Enhanced gene silencing of HIV-1 specific siRNA using microRNA designed hairpins. Nucleic Acids Res. 2004 Feb. 13; 32(3):1154-8. Print 2004.
Carroll R, Lin J T, Dacquel E J, Mosca J D, Burke D S, St Louis D C. A human immunodeficiency virus type 1 (HIV-1)-based retroviral vector system utilizing stable HIV-1 packaging cell lines. J Virol. 1994 September; 68(9):6047-51.
Chen J C, Krucinski J, Miercke L J, Finer-Moore J S, Tang A H, Leavitt A D, Stroud R M. Crystal structure of the HIV-1 integrase catalytic core and C-terminal domains: a model for viral DNA binding. Proc Natl Acad Sci USA. 2000 Jul. 18; 97(15):8233-8.
Chen J, Powell D, Hu W S. High frequency of genetic recombination is a common feature of primate lentivirus replication. J Virol. 2006 October; 80(19):9651-8.
Coffin J M. Structure, replication, and recombination of retrovirus genomes: some unifying hypotheses. J Gen Virol. 1979 January; 42(1):1-26.
Cornetta K, Pollok K E, Miller A D. Retroviral vector production by transient transfection. CSH Protoc. 2008 Apr. 1; 2008:pdb.prot4881. doi: 10.1101/pdb.prot4881.
Craig J C, Duncan I B, Hockley D, Grief C, Roberts N A, Mills J S. Antiviral properties of Ro 31-8959, an inhibitor of human immunodeficiency virus (HIV) proteinase. Antiviral Res. 1991 December; 16(4):295-305.
Craigie Robert. HIV Integrase, a Brief Overview from Chemistry to Therapeutics. J Biol Chem. 2001 Jun. 29; 276(26):23213-6
Creighton C J, Benham A L, Zhu H, Khan M F, Reid J G, Nagaraja A K, Fountain M D, Dziadek O, Han D, Ma L, Kim J, Hawkins S M, Anderson M L, Matzuk M M, Gunaratne P H. Discovery of novel microRNAs in female reproductive tract using next generation sequencing. PLoS One. 2010 Mar. 10; 5(3):e9637.
Cummings M D, Lin T I, Hu L, Tahri A, McGowan D, Amssoms K, Last S, Devogelaere B, Rouan M C, Vijgen L, Berke J M, Dehertogh P, Fransen E, Cleiren E, van der Helm L, Fanning G, Van Emelen K, Nyanguile O, Simmen K, Raboisson P, Vendeville S. Structure-based macrocyclization yields hepatitis C virus NS5B inhibitors with improved binding affinities and pharmacokinetic properties. Angew Chem Int Ed Engl. 2012 May 7; 51(19):4637-40. doi: 10.1002/anie.201200110. Epub 2012 Mar. 30.
Das A T, Brummelkamp T R, Westerhout E M, Vink M, Madiredjo M, Bernards R, Berkhout B. Human immunodeficiency virus type 1 escapes from RNA interference-mediated inhibition. J Virol. 2004 March; 78(5):2601-5.
Das K, Bauman J D, Clark A D Jr, Frenkel Y V, Lewi P J, Shatkin A J, Hughes S H, Arnold E. High-resolution structures of HIV-1 reverse transcriptase/TMC278 complexes: strategic flexibility explains potency against resistance mutations. Proc Natl Acad Sci USA. 2008 Feb. 5; 105(5):1466-71. Epub 2008 Jan. 29.
Equinones-Mateu, M E, and Arts, E J. Recombination in HIV-1: Update and Implications. AIDS Rev. 1999, 1, 89-100.
Fang G, Burger H, Chappey C, Rowland-Jones S, Visosky A, Chen C H, Moran T, Townsend L, Murray M, Weiser B. Analysis of transition from long-term nonprogressive to progressive infection identifies sequences that may attenuate HIV type 1. AIDS Res Hum Retroviruses. 2001 Oct. 10; 17(15):1395-404.
Feng Y, Zhang X, Song Q, Li T, Zeng Y. Drosha processing controls the specificity and efficiency of global microRNA expression. Biochim Biophys Acta. 2011 November-December; 1809(11-12):700-7. Epub 2011 Jun. 13.
Fiser A., Do R. K., Sali A. Modeling of loops in protein structures, Protein Science 9. 1753-1773, 2000.
Fraser C. HIV recombination: what is the impact on antiretroviral therapy? J R Soc Interface. 2005 Dec. 22; 2(5):489-503.
Freeman G A, Andrews Iii C W 3rd, Hopkins A L, Lowell G S, Schaller L T, Cowan J R, Gonzales S S, Koszalka G W, Hazen R J, Boone L R, Ferris R G, Creech K L, Roberts G B, Short S A, Weaver K, Reynolds D J, Milton J, Ren J, Stuart D I, Stammers D K, Chan J H. Design of non-nucleoside inhibitors of HIV-1 reverse transcriptase with improved drug resistance properties. 1. J Med Chem. 2004 Nov. 18; 47(24):5912-22.
Gamazon E R, Im H K, Duan S, Lussier Y A, Cox N J, Dolan M E, Zhang W. Exprtarget: an integrative approach to predicting human microRNA targets. PLoS One. 2010 Oct. 21; 5(10):e13534.
Gao K, Butler S L, Bushman F. Human immunodeficiency virus type 1 integrase: arrangement of protein domains in active cDNA complexes. EMBO J. 2001 Jul. 2; 20(13):3565-76.
Garcia J V, Alfano J, Miller A D. The negative effect of human immunodeficiency virus type 1 Nef on cell surface CD4 expression is not species specific and requires the cytoplasmic domain of CD4. J Virol. 1993 March; 67(3):1511-6.
Ge Q, Ilves H, Dallas A, Kumar P, Shorenstein J, Kazakov S A, Johnston B H. Minimal-length short hairpin RNAs: the relationship of structure and RNAi activity. RNA. 2010 January; 16(1):106-17. Epub 2009 Dec. 1.
Georgens C, Weyermann J, Zimmer A. Recombinant virus like particles as drug delivery system. Curr Pharm Biotechnol. 2005 February; 6(1):49-55.
Goldgur Y, Craigie R, Cohen G H, Fujiwara T, Yoshinaga T, Fujishita T, Sugimoto H, Endo T, Murai H, Davies D R. Structure of the HIV-1 integrase catalytic domain complexed with an inhibitor: a platform for antiviral drug design. Proc Natl Acad Sci USA. 1999 Nov. 9; 96(23):13040-3.
Griffiths-Jones S. The microRNA Registry. NAR 2004 32(Database Issue):D109-D111
Griffiths-Jones S, Grocock R J, van Dongen S, Bateman A, Enright A J. MicroRNA sequences, targets and gene nomenclature. NAR 2006 34(Database Issue):D140-D144
Griffiths-Jones S, Saini H K, van Dongen S, Enright A J. miRBase: tools for microRNA genomics. Nucleic Acids Res. 2008 January; 36 (Database issue):D154-8. Epub 2007 Nov. 8.
Guo S, Kemphues K J. par-1, a gene required for establishing polarity in C. elegans embryos, encodes a putative Ser/Thr kinase that is asymmetrically distributed. Cell. 1995 May 19; 81(4):611-20.
Haddrick M, Han-Liu Z, Lau A, Heaphy S, Cann A J. Production of non-infectious human immunodeficiency virus-like particles which package specifically viral RNA. J Virol Methods. 1996 September; 61(1-2):89-93.
Haffar O, Garrigues J, Travis B, Moran P, Zarling J, Hu S L. Human immunodeficiency virus-like, nonreplicating, gag-env particles assemble in a recombinant vaccinia virus expression system. J Virol. 1990 June; 64(6):2653-9.
Hanna Z, Priceputu E, Hu C, Vincent P, Jolicoeur P. HIV-1 Nef mutations abrogating downregulation of CD4 affect other Nef functions and show reduced pathogenicity in transgenic mice. Virology. 2006 Mar. 1; 346(1):40-52. Epub 2005 Nov. 28.
Haselhorst D, Kaye J F, Lever A M. Development of cell lines stably expressing human immunodeficiency virus type 1 proteins for studies in encapsidation and gene transfer. J Gen Virol. 1998 February; 79 (Pt 2):231-7.
Hemann M T, Fridman J S, Zilfou J T, Hernando E, Paddison P J, Cordon-Cardo C, Hannon G J, Lowe S W. An epi-allelic series of p53 hypomorphs created by stable RNAi produces distinct tumor phenotypes in vivo. Nat Genet. 2003 March; 33(3):396-400. Epub 2003 Feb. 3.
Hopkins A L, Ren J, Milton J, Hazen R J, Chan J H, Stuart D I, Stammers D K. Design of non-nucleoside inhibitors of HIV-1 reverse transcriptase with improved drug resistance properties. 1. J Med Chem. 2004 Nov. 18; 47(24):5912-22.
Huang H, Chopra R, Verdine G L, Harrison S C. Structure of a covalently trapped catalytic complex of HIV-1 reverse transcriptase: implications for drug resistance. Science. 1998 Nov. 27; 282(5394): 1669-75.
Hütter G, Nowak D, Mossner M, Ganepola S, Müssig A, Allers K, Schneider T, Hofmann J, Kücherer C, Blau O, Blau I W, Hofmann W K, Thiel E. Long-term control of HIV by CCR5 Delta32/Delta32 stem-cell transplantation. N Engl J Med. 2009 Feb. 12; 360(7):692-8.
Jacks T, Power M D, Masiarz F R, Luciw P A, Barr P J, Varmus H E. Characterization of ribosomal frameshifting in HIV-1 gag-pol expression. Nature. 1988 Jan. 21; 331(6153):280-3.
Jenkins T M, Esposito D, Engelman A, Craigie R. Critical contacts between HIV-1 integrase and viral DNA identified by structure-based analysis and photo-crosslinking. EMBO J. 1997 Nov. 17; 16(22):6849-59.
Julias J G, Ferris A L, Boyer P L, Hughes S H. Replication of phenotypically mixed human immunodeficiency virus type 1 virions containing catalytically active and catalytically inactive reverse transcriptase. J Virol. 2001 July; 75(14):6537-46.
Julias J G, Boyer P L, McWilliams M J, Alvord W G, Hughes S H. Mutations at position 184 of human immunodeficiency virus type-1 reverse transcriptase affect virus titer and viral DNA synthesis. Virology. 2004 Apr. 25; 322(1):13-21.
Karacostas V, Nagashima K, Gonda M A, Moss B. Human immunodeficiency virus-like particles produced by a vaccinia virus expression vector. Proc Natl Acad Sci USA. 1989 November; 86(22):8964-7.
Kaushik N, Rege N, Yadav P N, Sarafianos S G, Modak M J, Pandey V N. Biochemical analysis of catalytically crucial aspartate mutants of human immunodeficiency virus type 1 reverse transcriptase. Biochemistry. 1996 Sep. 10; 35(36):11536-46.
Kempf D J, Marsh K C, Denissen J F, McDonald E, Vasavanonda S, Flentge C A, Green B E, Fino L, Park C H, Kong X P, et al., ABT-538 is a potent inhibitor of human immunodeficiency virus protease and has high oral bioavailability in humans. Proc Natl Acad Sci USA. 1995 Mar. 28; 92(7):2484-8.
Khvorova A, Reynolds A, Jayasena S D. Functional siRNAs and miRNAs exhibit strand bias. Cell. 2003 Oct. 17; 115(2):209-16.
Kozomara A, Griffiths-Jones S. Integrating microRNA annotation and deep-sequencing data. NAR 2011 39(Database Issue):D152-D157
Lagos-Quintana M, Rauhut R, Lendeckel W, Tuschl T. Identification of novel genes coding for small expressed RNAs. Science. 2001 Oct. 26; 294(5543):853-8.
Lalezari J P, Eron J J, Carlson M, Cohen C, DeJesus E, Arduino R C, Gallant J E, Volberding P, Murphy R L, Valentine F, Nelson E L, Sista P R, Dusek A, Kilby J M. A phase II clinical study of the long-term safety and antiviral activity of enfuvirtide-based antiretroviral therapy. AIDS 2003 Mar. 28; 17(5):691-8.
Lau N C, Lim L P, Weinstein E G, Bartel D P. An abundant class of tiny RNAs with probable regulatory roles in Caenorhabditis elegans. Science. 2001 Oct. 26; 294(5543):858-62.
Lee Y, Ahn C, Han J, Choi H, Kim J, Yim J, Lee J, Provost P, RÃ¥dmark O, Kim S, Kim V N. The nuclear RNase III Drosha initiates microRNA processing. Nature. 2003 Sep. 25; 425(6956):415-9.
Li L, Lin X, Khvorova A, Fesik S W, Shen Y. Defining the optimal parameters for hairpin-based knockdown constructs. RNA. 2007 October; 13(10):1765-74. Epub 2007 Aug. 13.
Lim L P, Lau N C, Weinstein E G, Abdelhakim A, Yekta S, Rhoades M W, Burge C B, Bartel D P. The microRNAs of Caenorhabditis elegans. Genes Dev, 2003 17(8):991-1008
Lim L P, Lau N C, Garrett-Engele P, Grimson A, Schelter J M, Castle J, Bartel D P, Linsley P S, Johnson J M. Microarray analysis shows that some microRNAs downregulate large numbers of target mRNAs. Nature. 2005 Feb. 17; 433(7027):769-73. Epub 2005 Jan. 30.
Liu Y P, Westerink J T, Ter Brake O, Berkhout B. RNAi-inducing lentiviral vectors for anti-HIV-1 gene therapy. Methods Mol Biol. 2011;721:293-311.
Lu X, Yu Q, Binder G K, Chen Z, Slepushkina T, Rossi J, Dropulic B. Antisense-mediated inhibition of human immunodeficiency virus (HIV) replication by use of an HIV type 1-based vector results in severely attenuated mutants incapable of developing resistance. J Virol. 2004 July; 78(13):7079-88.
Marti-Renom M. A., Stuart A., Fiser A., Sanchez R., Melo F., Sali A. Comparative protein structure modeling of genes and genomes. Annu. Rev. Biophys. Biomol. Struct. 29, 291-325, 2000.
Matsumi S, Kosalaraksa P, Tsang H, Kavlick M F, Harada S, Mitsuya H. Pathways for the emergence of multi-dideoxynucleoside-resistant HIV-1 variants. AIDS. 2003 May 23; 17(8):1127-37.
Métifiot M, Maddali K, Naumova A, Zhang X, Marchand C, Pommier Y. Biochemical and pharmacological analyses of HIV-1 integrase flexible loop mutants resistant to raltegravir. Biochemistry. 2010 May 4; 49(17):3715-22.
Meyers B C, Axtell M J, Bartel B, Bartel D P, Baulcombe D, Bowman J L, Cao X, Carrington J C, Chen X, Green P J, Griffiths-Jones S, Jacobsen S E, Mallory A C, Martienssen R A, Poethig R S, Qi Y, Vaucheret H, Voinnet O, Watanabe Y, Weigel D, Zhu J K. Criteria for annotation of plant MicroRNAs. Plant Cell. 2008 December; 20(12):3186-90. Epub 2008 Dec. 12.
Min H, Yoon S. Got target? Computational methods for microRNA target prediction and their extension. Exp Mol Med. 2010 Apr. 30; 42(4):233-44.
Naito Y, Yamada T, Ui-Tei K, Morishita S, Saigo K. siDirect: highly effective, target-specific siRNA design software for mammalian RNA interference. Nucleic Acids Res. 2004 Jul. 1; 32(Web Server issue):W124-9.
Novina C D, Murray M F, Dykxhoorn D M, Beresford P J, Riess J, Lee S K, Collman R G, Lieberman J, Shankar P, Sharp P A. siRNA-directed inhibition of HIV-1 infection. Nat Med. 2002 July; 8(7):681-6. Epub 2002 Jun. 3.
Paddison P J, Caudy A A, Bernstein E, Hannon G J, Conklin D S. Short hairpin RNAs (shRNAs) induce sequence-specific silencing in mammalian cells. Genes Dev., 2002 16(8):948-58.
Pal N, Boyapalle S, Beckett R, Miller W A, Bonning B C. A baculovirus-expressed dicistrovirus that is infectious to aphids. J Virol. 2007 September; 81(17):9339-45. Epub 2007 Jun. 27.
Pauza C D, Galindo J E, Richman D D. Reinfection results in accumulation of unintegrated viral DNA in cytopathic and persistent human immunodeficiency virus type 1 infection of CEM cells. J Exp Med. 1990 Oct. 1; 172(4):1035-42.
Reuter J S, Mathews D H. RNAstructure: software for RNA secondary structure prediction and analysis. BMC Bioinformatics. 2010 Mar. 15; 11:129.
Rhodes D I, Peat T S, Vandegraaff N, Jeevarajah D, Le G, Jones E D, Smith J A, Coates J A, Winfield L J, Thienthong N, Newman J, Lucent D, Ryan J H, Savage G P, Francis C L, Deadman J J. Structural basis for a new mechanism of inhibition of HIV-1 integrase identified by fragment screening and structure-based design. Antivir Chem Chemother. 2011 Mar. 7; 21(4):155-68.
Saayman S, Arbuthnot P, Weinberg M S. Deriving four functional anti-HIV siRNAs from a single Pol III-generated transcript comprising two adjacent long hairpin RNA precursors. Nucleic Acids Res. 2010 October; 38(19):6652-63. Epub 2010 Jun. 4.
Saini H K, Enright A J, Griffiths-Jones S. Annotation of mammalian primary microRNAs. BMC Genomics. 2008 Nov. 27; 9:564.
Sali A., & Blundell T. L. Comparative protein modelling by satisfaction of spatial restraints. J. Mol. Biol. 234, 779-815, 1993.
Salminen M O, Koch C, Sanders-Buell E, Ehrenberg P K, Michael N L, Carr J K, Burke D S, McCutchan F E. Recovery of virtually full-length HIV-1 provirus of diverse subtypes from primary virus cultures using the polymerase chain reaction. Virology. 1995 Oct. 20; 213(1):80-6.
Schröder A R, Shinn P, Chen H, Berry C, Ecker J R, Bushman F. HIV-1 integration in the human genome favors active genes and local hotspots. Cell. 2002 Aug. 23; 110(4):521-9.
Schwarz D S, HutvÃ [symbol] gner G, Du T, Xu Z, Aronin N, Zamore P D. Asymmetry in the assembly of the RNAi enzyme complex. Cell. 2003 Oct. 17; 115(2):199-208.
Shehu-Xhilaga M, Crowe S M, Mak J. Maintenance of the Gag/Gag-Pol ratio is important for human immunodeficiency virus type 1 RNA dimerization and viral infectivity. J Virol. 2001 February; 75(4):1834-41.
Shen W, Gao L, Balakrishnan M, Bambara R A. A recombination hot spot in HIV-1 contains guanosine runs that can form a G-quartet structure and promote strand transfer in vitro. J Biol Chem. 2009 Dec. 4; 284(49):33883-93. Epub 2009 Oct. 12.
Simon-Loriere E, Martin D P, Weeks K M, Negroni M. RNA structures facilitate recombination-mediated gene swapping in HIV-1. J Virol. 2010 December; 84(24):12675-82. Epub 2010 Sep. 29.
Smith R A, Klarmann G J, Stray K M, von Schwedler U K, Schinazi R F, Preston B D, North T W. A new point mutation (P157S) in the reverse transcriptase of human immunodeficiency virus type 1 confers low-level resistance to (−)-beta-2′,3′-dideoxy-3′-thiacytidine. Antimicrob Agents Chemother. 1999 August; 43(8):2077-80.
Smith R A, Anderson D J, Preston B D. Purifying selection masks the mutational flexibility of HIV-1 reverse transcriptase. J Biol Chem. 2004 Jun. 18; 279(25):26726-34. Epub 2004 Mar. 9.
Smith R A, Anderson D J, Preston B D. Hypersusceptibility to substrate analogs conferred by mutations in human immunodeficiency virus type 1 reverse transcriptase. J Virol. 2006 July; 80(14):7169-78.
Stephenson M L, Zamecnik P C. Inhibition of Rous sarcoma viral RNA translation by a specific oligodeoxyribonucleotide. Proc Natl Acad Sci USA. 1978 January; 75(1):285-8.
Stoesser, G., Baker, W., van den Broek, A., Garcia-Pastor, M., Kanz, C., Kulikova, T., Leinonen, R., Lin, Q., Lombard, V., Lopez, R. et al., The EMBL nucleotide sequence database. Nucleic Acids Res. 2003 31, 17-22.
Tateno Y, Fukami-Kobayashi K, Miyazaki S, Sugawara H, Gojobori T. DNA Data Bank of Japan at work on genome sequence data. Nucleic Acids Res. 1998 Jan. 1; 26(1):16-20
Thompson J D. Applications of antisense and siRNAs during preclinical drug development. Drug Discov Today. 2002 Sep. 1; 7(17):912-7.
Tuschl, T. Expanding small RNA interference. Nat Biotechnol. 20(5):446-8. (2002)
Ui-Tei K, Naito Y, Takahashi F, Haraguchi T, Ohki-Hamazaki H, Juni A, Ueda R, Saigo K. Guidelines for the selection of highly effective siRNA sequences for mammalian and chick RNA interference. Nucleic Acids Res. 2004 Feb. 9; 32(3):936-48. Print 2004.
V Peremyslov V, V Dolja V. Cloning of large positive-strand RNA viruses. Curr Protoc Microbiol. 2007 November; Chapter 16:Unit 16F.1.
Wang X. miRDB: A microRNA target prediction and functional annotation database with a wiki interface. RNA. 2008 June; 14(6):1012-7. Epub 2008 Apr. 21.
Weng, L., Hirata, Y., Arai, M., Kohara, M., Wakita, T., Watashi, K., Shimotohno, K., He, Y., Zhong, J. and Toyoda, T. Sphingomyelin activates hepatitis C virus RNA polymerase in a genotype-specific manner. J. Virol. 84 (22), 11761-11770 (2010).
Wyatt J R, Vickers T A, Roberson J L, Buckheit R W Jr, Klimkait T, DeBaets E, Davis P W, Rayner B, Imbach J L, Ecker D J. Combinatorially selected guanosine-quartet structure is a potent inhibitor of human immunodeficiency virus envelope-mediated cell fusion. Proc Natl Acad Sci USA. 1994 Feb. 15; 91(4):1356-60.
Zamore P D. RNA interference: listening to the sound of silence. Nat Struct Biol. 2001 September; 8(9):746-50.
Zeng Y, Cullen B R. RNA interference in human cells is restricted to the cytoplasm. RNA. 2002 July; 8(7):855-60.
Zeng Y, Wagner E J, Cullen B R. Both natural and designed micro RNAs can inhibit the expression of cognate mRNAs when expressed in human cells. Mol Cell. 2002 June; 9(6): 1327-33.
Zeng Y, Cullen B R. Sequence requirements for micro RNA processing and function in human cells. RNA. 2003 January; 9(1):112-23.
Zeng Y, Yi R, Cullen B R. MicroRNAs and small interfering RNAs can inhibit mRNA expression by similar mechanisms. Proc Natl Acad Sci USA. 2003 Aug. 19; 100(17):9779-84. Epub 2003 Aug. 5.
Zeng Y, Cullen B R. Structural requirements for pre-microRNA binding and nuclear export by Exportin 5. Nucleic Acids Res. 2004 Sep. 8; 32(16):4776-85. Print 2004.
Zeng Y, Yi R, Cullen B R. Recognition and cleavage of primary microRNA precursors by the nuclear processing enzyme Drosha. EMBO J., 2005¹ Jan. 12; 24(1):138-48. Epub 2004 Nov. 25.
Zeng Y, Cai X, Cullen B R. Use of RNA polymerase II to transcribe artificial microRNAs. Methods Enzymol., (2005) 392:371-80.
Zeng Y, Cullen B R. Efficient processing of primary microRNA hairpins by Drosha requires flanking nonstructured RNA sequences. J Biol Chem. 2005 Jul. 29; 280(30):27595-603. Epub 2005 Jun. 1.

### Relevant Patents:

Gesteland, R F., Atkins, J F., Mateeva, O V, Shabalina, S A. (Inventors) Methods, Articles, And Compositions For Identifying Oligonucleotides. Publication date: Feb. 6, 2005. WO/2005/049851 Thermodynamic evaluations of oligonucleotides inter and intra molecular pairing potentials, hybridization intensity etc.
Drlica Karl, Wang Jian-Jing. (Inventors) Selection of target sites for the antisense attack of RNA. Publication date: 28 Nov. 2002. WO/2002/095059. Nucleic acid hybridization under steady conditions described by kinetic model.
Lever, Andrew., Chadwick, D R. (Inventors) Antiviral Antisense Therapy. Publication date: 19 Feb. 2003. WO/2001/090347 Antisense polynucleotides which bind the TAR region or splice-donor/packaging signal (SD/Ψ) region.
Jagneesh Pyati. (Inventor) Expression Constructs containing HIV inhibitory antisense and other nucleotide sequences, retroviral vectors and recombinant retroviruses containing same. Publication Date: 31 Aug. 1994. EP-0612844A2.
Administration of antisense constructs either traditional methods or use of recombinant retrovirus delivery systems. Modifying the envelop to suit the cells to which it should be delivered to. (May be the antisense was not given as pre-miRNAs?)
Corbeau, Pierre., Kraus, Gunter., and Wong-staal, Flossie. (Inventors) Design Of Novel Highly Efficient Hiv Based Packaging Systems For Gene Therapy. Publication date. 9 Oct. 1997. WO 97/36481
Friesen, A D. (Inventor) Antisense oligonucleotides and therapeutic use thereof in human immunodeficiency virus infection. Publication Date. 5 Jan. 1995. WO 95/00638.
Syngenix Limited (Applicant) Antiviral therapy. Application Date: 23 May 2000. Application Number. 0012497.4
Brahmachari, S K., Hariharan, M., Scaria, V., Pillai, B. (Inventors) Human microRNA Targets in HIV Genome and a Method of Identification Thereof. Publication Date. 19 Apr. 2007. WO 2007/042899

1. A therapeutic virus (TV), capable of inhibiting propagation of a target virus, the TV comprising:
a) an inactive essential gene for propagation of the TV in the host of the target virus or the absence of the gene essential for the propagation of TV in the host of the target virus, wherein the TV is adapted so that the TV can not propagate alone in the host of the target virus but the TV can propagate in the presence the target virus providing of an active form of the essential gene;
b) a sequence encoding a pre-miRNA, wherein an miRNA product from the pre-miRNA is adapted to have a first affinity for a highly conserved sequence of a gene in the target virus, whereby the miRNA inhibits translation of the highly conserved sequence; and,
c) a modified version of the highly conserved target sequence, which modified version is adapted to transcribe into an RNA transcript with a second affinity lower that the first affinity for the miRNA sequence;
whereby the TV is adapted to not propagate in the host cell without the presence of the target virus, and adapted so that the target virus can not propagate in the host cell in the presence of the TV.

2. The TV of claim 1, wherein the TV is deficient in at least two enzymes necessary for replication in a host cell for the target virus.

3. The TV of claim 1, wherein the TV is a modified version of the target virus, wherein the modifications comprise:
inactivation of a first essential gene product essential for propagation; and,
modification of the gene to increase mismatches to the miRNA.

4. The TV of claim 1, wherein the essential gene and the highly conserved sequence of a gene in the target virus are other than the same gene.

5. The TV of claim 1, wherein the essential gene and the highly conserved sequence of a gene in the target virus are the same gene, and the gene is a reverse transcriptase (RT), integrase, or an RNA dependent RNA polymerase (RDRP).

6. The TV of claim 1, wherein the target host cell encodes a functional reverse transcriptase, a functional RNA dependent RNA polymerase (RDRP), or a functional integrase enzyme, and the TV does not encode one or more of a functional reverse transcriptase, a functional RNA dependent RNA polymerase (RDRP), or a functional integrase enzyme.

7. The TV of claim 1, wherein the pre-miRNA is adapted to provide the miRNA product by the presence of Dicer or Drosha cutting sites in the pre-miRNA.

8. The TV of claim 1, wherein the miRNA, or pre-miRNA has at least 85% identity to at least one of the antisense sequences from the group consisting of:
UAUUGCUGGUGAUCCUUUCCA (SEQ ID NO: 1);
CUGUCCAUUUAUCAGGAUGGAG (SEQ ID NO: 2);
CCAAUCCCCCCUUUUCUUUUAAA (SEQ ID NO: 3);
AUACUGCCAUUUGUACUGCUGU (SEQ ID NO: 4); and,
a complementary sequence thereof.

9. A TV having at least 85% identity to the sequence of FIG. 30 and retaining at least 95% identity to underlined sequences, wherein the TV is adapted to inhibit replication of HIV when both the TV and HIV are present in the same cell.

10. A TV having at least 85% identity to the sequence of FIG. 36 or of FIG. 37, and retaining at least 95% identity to capitalized sequences, wherein the TV is adapted to inhibit replication of hepatitis type C (HCV) when both the TV and HCV are present in the same cell.

11. A method of inhibiting replication of a target virus, the method comprising:
a) providing a therapeutic virus (TV) comprising:
i) an inactive essential gene for propagation in the host of the target virus or the absence of the gene essential for the propagation of TV in the host of the target virus, whereby the TV can not propagate alone in the normal host of the target virus but the TV can propagate in the presence the target virus providing of an active form of the essential gene;
ii) a sequence encoding a pre-miRNA, wherein an miRNA product from the pre-miRNA is adapted to have a first affinity for a highly conserved sequence of the target virus, whereby the miRNA inhibits translation of the highly conserved sequence; and,
iii) a modified version of the highly conserved target sequence, which modified version is adapted to transcribe into an RNA transcript with a second affinity lower that the first affinity for the miRNA sequence; and,
b) contacting a target virus infected cell with the TV.

12. The method of claim 11, wherein the TV is adapted to be deficient in at least two enzymes necessary for replication in a host cell for the target virus.

13. The method of claim 11, wherein the TV is a modified version of the target virus, wherein the modifications are provided by:
inactivating a first essential gene product essential for propagation; and,
modifying the gene to increase mismatches to the miRNA.

14. The method of claim 11, wherein the essential gene for propagation and highly conserved sequence of the target virus are other than the same gene.

15. The method of claim 11, wherein the essential gene for propagation and highly conserved sequence of the target virus are the same gene, and the gene is a reverse transcriptase (RT), an integrase, or an RNA dependent RNA polymerase (RDRP).

16. The method of claim 11, further comprising converting the pre-miRNA into the miRNA by cutting with Cutter or Drosha.

17. The method of claim 11, further comprising inhibiting translation of the highly conserved sequence by hybridization of the miRNA to an mRNA of the target virus, which mRNA encodes the highly conserved sequence.

18. The method of claim 11, further comprising modifying the highly conserved target sequence to provide the modified version in the TV by changing codon triplet codes to alternate codons encoding the same amino acid.

19. The method of claim 11, further comprising adapting the miRNA to not bind to the modified TV highly conserved target sequence under intracellular host cell conditions.


SEQUENCE LISTING (97 sequences)

SEQ ID NO: 1 - RNA, 21 nt, Artificial
  miRNA designed against the first conserved site within RT
uauugcuggugauccuuucca

SEQ ID NO: 2 - RNA, 22 nt, Artificial
  miRNA designed against the second conserved site within RT
cuguccauuuaucaggauggag

SEQ ID NO: 3 - RNA, 23 nt, Artificial
  miRNA designed against first conserved site within integrase,
ccaauccccccuuuucuuuuaaa

SEQ ID NO: 4 - RNA, 22 nt, Artificial
  miRNA designed against second conserved site within integrase.
auacugccauuuguacugcugu

SEQ ID NO: 5 - DNA, 21 bp, Artificial
  miRNA designed against the first conserved site within RT
tattgctggtgatcctttcca

SEQ ID NO: 6 - DNA, 22 bp, Artificial
  miRNA designed against the second conserved site within RT
ctgtccatttatcaggatggag

SEQ ID NO: 7 - DNA, 23 bp, Artificial
  miRNA designed against the first conserved site within integrase
ccaatccccccttttcttttaaa

SEQ ID NO: 8 - DNA, 23 bp, Artificial
  miRNA designed against the first conserved site within integrase
ccaatccccccttttcttttaaa

SEQ ID NO: 9 - DNA, 22 bp, Artificial
  miRNA designed against the second conserved site within integrase
atactgccatttgtactgctgt

SEQ ID NO: 10 - DNA, 22 bp, Artificial
  miRNA designed against the first conserved site within NS5B
tattgatttcacctggagagta

SEQ ID NO: 11 - DNA, 24 bp, Artificial
  raw antisense developed for the first conserved site within RT
atattgctggtgatcctttccatc

SEQ ID NO: 12 - DNA, 22 bp, Homo sapiens
tgcggggacaggccagggcatc

SEQ ID NO: 13 - DNA, 22 bp, Homo sapiens
atattattagccacttctggat

SEQ ID NO: 14 - DNA, 21 bp, Artificial
  miRNA designed against the first conserved site within RT
tattgctggtgatcctttcca

SEQ ID NO: 15 - DNA, 18 bp, Homo sapiens
ctggagtctaggattcca

SEQ ID NO: 16 - DNA, 22 bp, Homo sapiens
tattgcacattactaagttgca

SEQ ID NO: 17 - DNA, 22 bp, Human immunodeficiency virus
ctccatcctgataaatggacag

SEQ ID NO: 18 - DNA, 22 bp, Artificial
  miRNA designed against the second conserved site within RT
ctgtccatttatcaggatggag

SEQ ID NO: 19 - DNA, 23 bp, Homo sapiens
tgcagctctggtggaaaatggag

SEQ ID NO: 20 - DNA, 23 bp, Homo sapiens
ctgtaatataaatttaatttatt

SEQ ID NO: 21 - DNA, 24 bp, Human immunodeficiency virus
ccaatccccccttttcttttaaaa

SEQ ID NO: 22 - DNA, 22 bp, Human immunodeficiency virus
ccaatccccccttttcttttaa

SEQ ID NO: 23 - DNA, 22 bp, Human immunodeficiency virus
caatccccccttttcttttaaa

SEQ ID NO: 24 - DNA, 23 bp, Human immunodeficiency virus
ccaatccccccttttcttttaaa

SEQ ID NO: 25 - DNA, 22 bp, Artificial
  miRNA designed against second conserved site within integrase.
atactgccatttgtactgctgt

SEQ ID NO: 26 - DNA, 22 bp, Artificial
  miRNA designed against the first conserved site within NS5B
tattgatttcacctggagagta

SEQ ID NO: 27 - DNA, 9217 bp, Human immunodeficiency virus
  ref <308>: DATABASE ACCESSION NUMBER: U69584
  ref <309>: DATABASE ENTRY DATE: 2003-04-28
  ref <313>: RELEVANT RESIDUES IN SEQ ID NO: (1)..(9217)
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagagaaaccagagaagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatagatgggaaaaaattcggctaaggccagggggaaagaaaaaatataaactaaaacatatagtatgggcaagcagggagctagaacgattcgcagttaatcctggcctgttagaaacatcagaaggctgtagacaaatactgggacagctacaaccagcccttcagacaggatcagaagaacttagatcattacataatacagtagcagtcctctattgtgttcatcaaaggatagaggtaaaagacaccaaggaagctttagagaagatagaggaagagcaaaacaaaagtaagaaaaaagcacagcaagcagcagctgacacaggaaacagcagcaaggtcagccaaaattaccctatagtgcagaacatccaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaactgcagaatgggatagattgcatccagtgcatgcagggcctattgcaccaggccagatgaacacagtggggggacatcaagcagccatgcaaatgttaaaagagaccatcaatgaggaaggagaaccaaggggaagtgacatagcaggaactactagtacccttcaggaacaaataggatggatgacaaataatccacctatcccagtaggagaaatttataaaagatggataatcctgggattaaataaaatagtaaggatgtatagtcctaccagcattctggacataagacaaggaccaaaggaaccctttagagactatgtagaccggttctataaaactctaagagccgagcaagcttcacaggaggtaaaaaattggatgacagaaaccttgttggtccaaaattcgaacccagattgtaagactattttaaaagcattgggaccagcagctacactagaagaaatgatgacagcatgtcagggagtgggaggacccagccataaagcaagagttttggctgaagcaatgagccaagcaacaagttcagctaccataatgatgcagagaggcaattttaggaaccaaagaaagattgttaagtgttttaattgtggcaaggaagggcacattgccagaaattgcagggcccctaggaaaaagggctgttggaaatgtggaaaggaaggacaccaaatgaaagactgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggagcagtatcctttagcttccctcagatcactctttggcaacgacccctcgtctcaataaagataggggggcaactaaaggaagctctattagatacaggagcagatgatacagtattagaagaaatgaatttgccaggaagatggaaaccaaaaatgatagggggaattggaggttttatcaaagtaagacagtatgatcagatactcgtagaaatctgtggacataaagctataggtacagtattagtaggacctacacccgtcaacataattggaagaaatctgttgactcagattggttgcactttaaattttcccattagtcctattgaaactgtaccagtaaaattaaagccaggaatggatggcccaaaagttaaacaatggccattgacagaagaaaaaataaaagcattagtagacatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaagaagaaaaaatcagtaacagtactggatgtgggtgatgcatatttttcagttcccttagataaagacttcagaaagtatcctgcatttaccatacctagtgtaaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatagatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaagttagtgggaaaattgaattgggcaagtcagatttacgcagggattaaagtaaagcaattatgtaaactccttaggggagccaaagcactaacagaagtaataccactaacagaagaagcagagctagaactggcagaaaatagagagattctaaaagaaccagtacatggagtgtattatgacccatcaaaagacttaatagcagaaatacagaagcaagggcaaggccaatggacatatcaaatttatcaagagccatataaaaatctgaaaacaggaaaatatgcaagaatgaggggtgcccacactaatgatgtaaaacaattaacagaggcagtgcaaaaaatagccacagaaagcatagtaatatggggaaagactcctaaatttaaactacccatacaaaaagaaacatgggaaacatggtggatagagtattggcaagcaacctggattcctgagtgggagtttgtcaatacccctcccttagtgaaattatggtaccagttagagaaagaacccatagtaggagcagaaactttctatgtagatggggcagctaacagggaaactaaattaggaaaagcaggatatgttactgacagaggaagacaaaaagttgtctccctaactgacacaacaaatcagaaaactgagttacaagcaattcatctagctttggaggattcgggcttagaagtaaacatagtaacagactcacaatatgcattaggaatcattcaagcacaaccagataagagtgaatcagagttagtcagtcaaataatagagcagttaataaaaaaggaaaaggtctacctggcatgggtaccagcacacaaaggaattggaggaaatgaacaagtagataaattagtcagtgctggaatcaggaaagtactatttttagatggaatagataaggcccaagaagaacatgagaaatatcacagtaattggagagcaatggctggtgattttaacctgccacctgtagtagcaaaagaaatagtagccagctgtgataaatgtcagctaaaaggagaagccatgcatggacaagtagactgtagtccaggaatatggcaactagattgtccacatttagaaggaaaagttatcctggtagcagttcatgtagccagtggatatatagaagcagaagttattccagcagagacagggcaggaaacagcatactttctcttaaaattagcaggaagatggccagtaaaaacaatacatgcagacaatggcagcaatttcaccagtactacagttaaggccgcctgttggtgggcagggatcaaacaggaatttggcattccctacaatccccaaagtcaaggagtagtagaatctatgaataaagaattaaagaaaattataggacaggtaagagatcaggctgaacatcttaagacagcagtccaaatggcagtattcatccacaattttaagagaaaaggggggattggggggtacagtgcaggggaaagaatagtagacataatagcaacagacatacaaactaaagaattacaaaaacatattacaaaaattcaacattttcgggtttattacagagacagcagagatccactttggaaaggaccagcaaagcttctctggaaaggtgaaggggcagtagtaatacaagataatagtgacataaaagtagtaccaagaagaaaagtaaagatcattagggattatggaaaacagatggcaggtgatgattgtgtggcaagtagacaggatgaggattagaacatggaaaagtttagtaaaacaccatatgtatgtttcagggaaagctaagaaatggttttatagacatcactatgaaaacactcatcctagaataagttcagaagtgcacatcccactaggggatgctaaattggtaatagcaacatattggggtctgcatacaggagaaagagactggcatttgggccagggagtctccatagaatggaggaaaaagagatatagcacacaagtagaccctggcctagcagaccaactaattcatctgtattattttgattgtttttcagaatctgctataagaaataccatattaggacatagagttagtcctagttgtgaatatcaagcaggacataacaaggtaggatctccacaatacttggcactagctgcattaatagcaccaaaaaagataaagccacctttgcctagtgttacgaaactgacagaggatagatggaacaagccccagaagaccaagggccacagagggagccatacaatgaatggacactagagcttttagaggagcttaagagtgaagctgttaggcattttcctaggttatggctccatagcttaggacaacatatctatgaaacttatggggatacttgggcaggagtggaagccataataagaattctgcaacaactgctgtttattcatttcagaattgggtgtcgacatagcagaataggcataattcgacagaggagagcaagaaatggagccagtagatcctagactagagccctggaagcatccaggaagtcagcctaggactgcttgcaccaattgctattgtaaaaagtgttgccttcattgccaagtttgtttcacaagaaaaggcttaggcatctcctatggcaggaagaagcggagacagcgacgaagagctcctcaagacagtcagactcatcaagcttctctatcaaagcagtaagtagtacatgtaatgcaatctttagaaatattagcaccagtagcattagtagtagcaacaataatagtaatagttgtgtggaccatagtattcatagaatataggaaactattaagacaaagaaaaatagacaggttaattgatagaataagagaaagagcagaagacagtggcaatgaaagcgaaggagaccaggaagaattatcagcacttgtggagatggggcatcacgctccttgggatgttgatgatctgtagtgctgcagaacaattgtgggtcacagtctattatggggtacctgtgtggaaagaagcaaccaccaccctattttgtgcatcagatgctaaagcatatgatacagaggtgcataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaagttaaccccactctgtgttactttaaattgcactgatgatttaaaaaatgctactgtaaaaaatgctactaataccaataatagtagctggggagggatggagagaggagaaataaaaaactgctctttcaatatcaccactagcataagagataaggtgcagaaagaatatgcacttttttataaacttgatgtagtaccaatagataatgctgataataataatattactactaactataccagctatagattgataagttgtaacacctcagtcattacacaggcctgtccaaaggtatcctttgagccaattcccatacattattgtgccccggctggttttgcgattctaaagtgtaacgataagaagttcaatggaacaggaccatgtacaaatgtcagcacagtacaatgtacacatggaattaggccagtagtatcaactcaactgctgttaaatggcagtctagcagaaaaagaggtagtaattagatctgaaaatttcacgaacaatgccaaaaccataatagtacagctggatgaatctgtagtaattaattgtacaagacccaacaacaatacaagaaaaagtatacatataggaccagggagagcattttatacaacaggagaaataataggagatataagacaagctcattgtacccttaatagaacagaatggaataacactttagcaaagataactgaaaaattaagagaacaatttgggaataacataacaatagtctttaatcactcctcgggaggggacccagaaattgtaatgcacagttttatttgtggaggggaatttttctactgtaatacatcacaactgtttaatagtacttggaatagtactgggaataatattagcgagtcagataacactgaaagaaatatcacactcccatgcagaataaaacaaattataaacctgtggcaggaagtaggaaaagcaatgtatgcccctcccatcagaggacaaattagatgttcatcaaatattacagggctgctattaacaagagatggtggtagtaacacggacgagaacaggaccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaacaataggagctatgttccttgggttcttgggagcagcaggaagcactatgggcgcagcgtcgatgacgctgacggtacaggccagactattattgtctggtatagtgcaacagcagaacaatttgctgagggctattgaggcgcaacagcatctgttgcaactcacagtctggggcatcaagcagctccaggcaagagtcctggctgtggaaagatacctaaaggatcaacagctcctagggatttggggttgctctggaaaactcatttgcaccactgctgtgccttggaatgctagttggagtaataaatctctggatgagatttgggataacatgacctggatggaatgggaaagagaaattgacaattacacaaacctaatatacaccttaattgaagaataccggaaccagcaagaaaagaatgaacaagcattattggaattagataaatgggcaagtttgtggaattggtttaacataacaaattggctgtggtatataaaaatattcataatgatagtaggaggcttggtaggtttaagaatagtctttactgtactttctatagtgaatagagttaggaagggatactcaccattatcgtttcagacccgcctcccaaccccgaggggacccgacaggcccgaaggaatcgaagaagaaggtggagagcgagacagagacagatccggtctcttagtggatggattcttagcacttatctgggtcgacctgcggagcctgtgcctcttcagctaccaccgattgagagacttactcttgattgtagcgaggattgtggaacttctgggacgcagggggtgggaagtcctcaaatattggtggaatctcctgcagtattggagtcaggaactaaagaatagtgcagttagcttgctcaatgccacagctatagcagtagctgaggggacagatagggttatagaagtattacaaagagcttgtagagctatcctccacatacctacaagaataagacagggcttggaaagggctttgctataagatgggtggcaagtggtcaaaacgtagtggggttggatggcctactgtaagggaaagaatgagacgagctgagccagcagcagatggggtgggagcagtatctcgagacctggaaaaacatggagcaatcacaagtagcaatacagcagctactaatgctgattgtgcctggctagaagcacaagaggaggaagaggtgggttttccagtcagacctcaggtacctttaagaccaatgacttacaagggagcgttagatcttagccactttttaagagaaaaggggggactggaagggctaatttactcccagaaaagacaagatatccttgatctgtgggtctaccacacacaaggcttcttccctgattggcagaactacacaccagggccagggatcagatacccactgtgttttggatggtgctttaagctagtaccagttgagccagagaaggtagaagaggccaatgaaggagagaacaacagcttgttacaccctatgagcctgcatgggatggaggacccggagaaagaagtgttagtgtggaggtttgacagccgcctagcagttcatcacatggcccgagagcagcatccggagtattacaagaactgctgacatcgagctttctacaagggactttccgctggggactttccacggaggtgtggccggggcgggactggggagtggcgaaccctcagatgctgcatataagcagctgctttttgcctgtactgggtctctctggttagaccagatctgagcctgggagctctctggctaactagggaacccactgcttaagcctcaataaagcttgccttgagtgcttca

SEQ ID NO: 28 - DNA, 22 bp, Human immunodeficiency virus
tggaaaggatcaccagcaatat

SEQ ID NO: 29 - protein, 7 aa, Human immunodeficiency virus
WKGSPAI

SEQ ID NO: 30 - DNA, 24 bp, Human immunodeficiency virus
atattgctggtgatcctttccatc

SEQ ID NO: 31 - DNA, 27 bp, Human immunodeficiency virus
ggatggaaaggatcaccagcaatattc

SEQ ID NO: 32 - protein, 9 aa, Human immunodeficiency virus
GWKGSPAIF

SEQ ID NO: 33 - DNA, 21 bp, Artificial
  modified RT sequence
tggacaagcaacagtgtgcta

SEQ ID NO: 34 - protein, 7 aa, Artificial
  modified RT sequence
WTSNSVL

SEQ ID NO: 35 - DNA, 83 bp, Homo sapiens
tctgggggttctggagtctaggattccaggatctgggttttgaggtcttgggttgtagggtctgcggtttgaagcccctcttg

SEQ ID NO: 36 - DNA, 70 bp, Homo sapiens
ggagatattgcacattactaagttgcatgttgtcacggcctcaatgcaatttagtgtgtgtgatattttc

SEQ ID NO: 37 - DNA, 210 bp, Homo sapiens
cagcatgttgtctcctgaatgtatacatcacacccttcttccctgatgctcccattctgcttgctctggtggagatattgcacattactaagttgcatgttgtcacggcctcaatgcaatttagtgtgtgtgatattttcacatgagtgcatgcacacgggtatggctcttgcttgctgggattacagcttttcagtgccagtgggtggg

SEQ ID NO: 38 - DNA, 211 bp, Artificial
  designed shRNA
cagcatgttgtctcctgaatgtatacatcacacccttcttccctgatgctcccattctgcttgctctggtggagatattgctggtgatcctttccaggatctgggttttgaggtcttgggaggatcaccagtgatattttcacatgagtgcatgcacacgggtatggctcttgcttgctgggattacagcttttcagtgccagtgggtggg

SEQ ID NO: 39 - DNA, 1674 bp, Human immunodeficiency virus
  CDS (1)..(1674)
cccattagtcctattgaaactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagtctaaagacagtactaaatggcttaaattagtagatttcagagaacttaataagagaactcaagacttctgggaagttcaattaggaataccacatcccgcagggtggaaaaagcagaaatcagagacagtactggaagtgggtgatgcatatttttcagttcccttagataaagacttcaggaagtatactgcatttaccatacctagtataaacaatgagacaccagggattagatatcagtacaatgtgcttgcattttggacaagcaacagtgtgctacaaagtagcatgacaaaaatcttagagccttttagaaaacaaaatccagacatagagatcacccaatacatgacaatattgctcgtaggatctgacttagaaatagggcagcatagaacaaaaatagaggaactgagacaacatctgttgaggtggggatttaccacaccagacaaaaaacatcagaaagaacctccattcctttggatgggttatgaaaaacatcctgataaatggacagtacagcctatagtgctgccagaaaaagacagctggactgtcaatgacatacagaagttagtgggaaaattgaattgggcaagtcagatttatgcagggattaaagtaaagcaattatgtaaactccttaggggaaccaaagcactaacagaagtagtaccactaacagaagaagcagagctagaactggcagaaaacagggaaattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaatagccacagaaagcatagtaatatggggaaagactcctaaatttaaactacccatacaaaaagaaacatgggaaacatggtggacagagtattggcaagccacctggattcctgagtgggagtttgtcaatacccctcccttagtgaaattatggtaccagttagagaaagaacccatagtaggagcagaaactttctatgtagatggggcagctaatagggagactaaattaggaaaagcaggatatgttactgacagaggaagacaaaaagttgtctccctaactgacacaacaaatcagaagactgagttacaagcaattcatctagctttgcaggattcaggattagaagtaaacatagtaacagactcacaatatgcattaggaatcattcaagcacaaccagataaaagtgaatcagagttagtcagtcaaataatagagcagttaataaaaaaggaaaaggtctacctggcatgggtaccagcacacaaaggaattggaggaaatgaacaagtagataaattagtcagtgctggaatcaggaaagtacta

SEQ ID NO: 40 - protein, 558 aa, Human immunodeficiency virus
PISPIETVPVKLKPGMDGPKVKQWPLTEEKIKALVEICTEMEKEGKISKIGPENPYNTPVFAIKSKDSTKWLKLVDFRELNKRTQDFWEVQLGIPHPAGWKKQKSETVLEVGDAYFSVPLDKDFRKYTAFTIPSINNETPGIRYQYNVLAFWTSNSVLQSSMTKILEPFRKQNPDIEITQYMTILLVGSDLEIGQHRTKIEELRQHLLRWGFTTPDKKHQKEPPFLWMGYEKHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWETWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVL

SEQ ID NO: 41 - DNA, 22 bp, Human immunodeficiency virus
ctccatcctgataaatggacag

SEQ ID NO: 42 - protein, 8 aa, Human immunodeficiency virus
LHPDKWTV

SEQ ID NO: 43 - DNA, 21 bp, Artificial
  antisense designed against RT
tgtccatttatcaggatggag

SEQ ID NO: 44 - DNA, 21 bp, Human immunodeficiency virus
catcctgataaatggacagta

SEQ ID NO: 45 - protein, 8 aa, Human immunodeficiency virus
  MISC_FEATURE (1)..(8)
LHPDKWTV

SEQ ID NO: 46 - DNA, 24 bp, Artificial
  modified RT sequence
aagaggagcacgtcctggtctttc

SEQ ID NO: 47 - protein, 7 aa, Artificial
  modified RT sequence
RSTSWSL

SEQ ID NO: 48 - DNA, 80 bp, Homo sapiens
aaactccttctgcagctctggtggaaaatggagaagacttttcctttcctccatctcccccagggcctggtggagtgagg

SEQ ID NO: 49 - DNA, 189 bp, Homo sapiens
atccatgagtttttgctaggtttcctttggcaacaattaagaagcaccagtgtgtagattctccttcttcctgtaatataaatttaatttattctctatcattaaaaaatgtattacagaggagcatattaaaatattcaacagagggtaattagctgtctatgttttatatgcagtaataatgcttga

SEQ ID NO: 50 - DNA, 199 bp, Artificial
  designed miRNA/shRNA
atccatgagtttttgctaggtttcctttggcaacaattaagaagcaccagtgtgtagattctccttcttcctgtccatttatcaggatggagaagacttttcctttcctccatcctgataaatggacagaggagcatattaaaatattcaacagagggtaattagctgtctatgttttatatgcagtaataatgcttga

SEQ ID NO: 51 - DNA, 1680 bp, Homo sapiens
  CDS (1)..(1680)
cccattagtcctattgaaactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagtctaaagacagtactaaatggcttaaattagtagatttcagagaacttaataagagaactcaagacttctgggaagttcaattaggaataccacatcccgcagggtggaaaaagcagaaatcagagacagtactggaagtgggtgatgcatatttttcagttcccttagataaagacttcaggaagtatactgcatttaccatacctagtataaacaatgagacaccagggattagatatcagtacaatgtgcttgcatttggatggaaaggatcaccagcaatattccaaagtagcatgacaaaaatcttagagccttttagaaaacaaaatccagacatagagatcacccaatacatgacaatattgctcgtaggatctgacttagaaatagggcagcatagaacaaaaatagaggaactgagacaacatctgttgaggtggggatttaccacaccagacaaaaaacatcagaaagaacctccattcctttggatgggttatgaaaagaggagcacgtcctggtctttccagcctatagtgctgccagaaaaagacagctggactgtcaatgacatacagaagttagtgggaaaattgaattgggcaagtcagatttatgcagggattaaagtaaagcaattatgtaaactccttaggggaaccaaagcactaacagaagtagtaccactaacagaagaagcagagctagaactggcagaaaacagggaaattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaatagccacagaaagcatagtaatatggggaaagactcctaaatttaaactacccatacaaaaagaaacatgggaaacatggtggacagagtattggcaagccacctggattcctgagtgggagtttgtcaatacccctcccttagtgaaattatggtaccagttagagaaagaacccatagtaggagcagaaactttctatgtagatggggcagctaatagggagactaaattaggaaaagcaggatatgttactgacagaggaagacaaaaagttgtctccctaactgacacaacaaatcagaagactgagttacaagcaattcatctagctttgcaggattcaggattagaagtaaacatagtaacagactcacaatatgcattaggaatcattcaagcacaaccagataaaagtgaatcagagttagtcagtcaaataatagagcagttaataaaaaaggaaaaggtctacctggcatgggtaccagcacacaaaggaattggaggaaatgaacaagtagataaattagtcagtgctggaatcaggaaagtacta

SEQ ID NO: 52 - protein, 560 aa, Homo sapiens
PISPIETVPVKLKPGMDGPKVKQWPLTEEKIKALVEICTEMEKEGKISKIGPENPYNTPVFAIKSKDSTKWLKLVDFRELNKRTQDFWEVQLGIPHPAGWKKQKSETVLEVGDAYFSVPLDKDFRKYTAFTIPSINNETPGIRYQYNVLAFGWKGSPAIFQSSMTKILEPFRKQNPDIEITQYMTILLVGSDLEIGQHRTKIEELRQHLLRWGFTTPDKKHQKEPPFLWMGYEKRSTSWSFQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWETWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVL

SEQ ID NO: 53 - DNA, 1674 bp, Human immunodeficiency virus; identical to SEQ ID NO: 39
  CDS (1)..(1674)

SEQ ID NO: 54 - protein, 558 aa, Human immunodeficiency virus; identical to SEQ ID NO: 40

SEQ ID NO: 55 - DNA, 24 bp, Human immunodeficiency virus
ttttaaaagaaaaggggggattgg

SEQ ID NO: 56 - protein, 8 aa, Human immunodeficiency virus
FKRKGGIG

SEQ ID NO: 57 - DNA, 24 bp, Artificial
  antisense to first conserved stretch within integrase
ccaatccccccttttcttttaaaa

SEQ ID NO: 58 - DNA, 24 bp, Artificial
  first conserved sretch of integrase, modified to interfere with binding of antisense
ggccgtcggggcaaagaacgagag

SEQ ID NO: 59 - protein, 8 aa, Artificial
  first conserved sretch of integrase, modified to interfere with binding of antisense
GRRGKERE

SEQ ID NO: 60 - DNA, 69 bp, Homo sapiens
tttatatgtaggcctttagatcacttaaagagtattcaacatcagatgatctaaaggcctatacataaa

SEQ ID NO: 61 - DNA, 205 bp, Homo sapiens
agacttcaggaggaagctgctttcactacttttttttttttaattaccatgagggttagaacatttttgtccaattaccacttctttttgccacctgagcacagtcagcagtcagcataaaaaagtgataatgggaagttaatgtctaaccaggtcacatttcgcttcaaaggaacaaaacacatacaaaatgcatgtttttttc

SEQ ID NO: 62 - DNA, 78 bp, Artificial
  shRNA designed against integrase
acatttttgtccaatccccccttttcttttaaagagtattcaacatcaaagaaaaggggggatgggaagttaatgtct

SEQ ID NO: 63 - DNA, 196 bp, Artificial
  pre-miRNA designed against integrase
agacttcaggaggaagctgctttcactacttttttttttttaattaccatgagggttagaacatttttgtccaatccccccttttcttttaaagagtattcaacatcaaagaaaaggggggatgggaagttaatgtctaaccaggtcacatttcgcttcaaaggaacaaaacacatacaaaatgcatgtttttttc

SEQ ID NO: 64 - DNA, 867 bp, Artificial
  modified HIV-1 integrase with mutations to inactivate and avoid binding of designed antisense
  CDS (1)..(867)
tttttagatggaatagataaggcccaagaagaacatgaaaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactaggctgtgtgcatttagaaggaaaaattatcctggtagcagttcatgtagccagtggatatatagaagcagaagttattccagcagaaacagggcaggaaacagcatactttatcttaaaattagcaggaagatggccagtaaaaacaatacatacaggtaatggcagcaatttcaccagtactgcagttaaggccgcctgttggtgggcagggatcaagcaggaatttggcattccctacaatccccaaagtcaaggagtagtacgttctatgaataaagaattacgtaaaattataggacaggtaagagatcaggctgaacatcttaagacagcagtacaaatggcagtattcattcacaacggccgtcggggcaaagaacgagaggggtacagtgcaggggaaagaataatagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 65 - protein, 288 aa, Artificial
  modified HIV-1 integrase
FLDGIDKAQEEHEKYHSNWRAMASDFNLPPVVAKEIVASCDKCQLKGEAMHGQVDCSPGIWQLGCVHLEGKIILVAVHVASGYIEAEVIPAETGQETAYFILKLAGRWPVKTIHTGNGSNFTSTAVKAACWWAGIKQEFGIPYNPQSQGVVRSMNKELRKIIGQVRDQAEHLKTAVQMAVFIHNGRRGKEREGYSAGERIIDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 66 - DNA, 24 bp, Human immunodeficiency virus
acagcagtacaaatggcagtattc

SEQ ID NO: 67 - protein, 8 aa, Human immunodeficiency virus
TAVQMAVF

SEQ ID NO: 68 - DNA, 22 bp, Artificial
  antisense designed against integrase
atactgccatttgtactgctgt

SEQ ID NO: 69 - DNA, 24 bp, Artificial
  modified integrase
cgtacgactttgctagtcgctcat

SEQ ID NO: 70 - protein, 8 aa, Artificial
  modified integrase
RTTLLVGH

SEQ ID NO: 71 - DNA, 71 bp, Homo sapiens
ctcacaagatcaatcacttggtaattgctgtgataacaactcagcaattaccaagtgattggttttgtgag

SEQ ID NO: 72 - DNA, 75 bp, Homo sapiens
gtttaaagaatactgtgaatttcactgtcacaaattcaaataaagtgagagtggaattcacagtatttaaggaat

SEQ ID NO: 73 - DNA, 71 bp, Artificial
  shRNA against integrase
gtttaaagaatactgccatttgtactgctgtgataacaactcagcagtacaaatggcagtatttaaggaat

SEQ ID NO: 74 - DNA, 195 bp, Homo sapiens
ataaaattattaggacaggcaaagcaattcagaggaaagagcatcgcttgggggtaaaatgtttaaagaatactgtgaatttcactgtcacaaattcaaataaagtgagagtggaattcacagtatttaaggaatcaatccttgctgcttctgtttatgaagccgagcttcagttttcttgctctgacaatggac

SEQ ID NO: 75 - DNA, 191 bp, Artificial
  pre-miRNA against integrase
ataaaattattaggacaggcaaagcaattcagaggaaagagcatcgcttgggggtaaaatgtttaaagaatactgccatttgtactgctgtgataacaactcagcagtacaaatggcagtatttaaggaatcaatccttgctgcttctgtttatgaagccgagcttcagttttcttgctctgacaatggac

SEQ ID NO: 76 - DNA, 867 bp, Artificial
  modified HIV-1 integrase with mutations to inactivate and avoid binding of designed antisense
  CDS (1)..(867)
tttttagatggaatagataaggcccaagaagaacatgaaaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactaggctgtgtgcatttagaaggaaaaattatcctggtagcagttcatgtagccagtggatatatagaagcagaagttattccagcagaaacagggcaggaaacagcatactttatcttaaaattagcaggaagatggccagtaaaaacaatacatacaggtaatggcagcaatttcaccagtactgcagttaaggccgcctgttggtgggcagggatcaagcaggaatttggcattccctacaatccccaaagtcaaggagtagtacgttctatgaataaagaattacgtaaaattataggacaggtaagagatcaggctgaacatcttaagcgtacgactttgctagtcgctcatattcacaattttaaaagaaaaggggggattggggggtacagtgcaggggaaagaataatagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 77 - protein, 288 aa, Artificial
  modified HIV-1 integrase
FLDGIDKAQEEHEKYHSNWRAMASDFNLPPVVAKEIVASCDKCQLKGEAMHGQVDCSPGIWQLGCVHLEGKIILVAVHVASGYIEAEVIPAETGQETAYFILKLAGRWPVKTIHTGNGSNFTSTAVKAACWWAGIKQEFGIPYNPQSQGVVRSMNKELRKIIGQVRDQAEHLKRTTLLVAHIHNFKRKGGIGGYSAGERIIDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 78 - DNA, 867 bp, Artificial
  modified HIV-1 integrase with mutations to inactivate and avoid binding of designed antisense
  CDS (1)..(867)
tttttagatggaatagataaggcccaagaagaacatgaaaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactaggctgtgtgcatttagaaggaaaaattatcctggtagcagttcatgtagccagtggatatatagaagcagaagttattccagcagaaacagggcaggaaacagcatactttatcttaaaattagcaggaagatggccagtaaaaacaatacatacaggtaatggcagcaatttcaccagtactgcagttaaggccgcctgttggtgggcagggatcaagcaggaatttggcattccctacaatccccaaagtcaaggagtagtacgttctatgaataaagaattacgtaaaattataggacaggtaagagatcaggctgaacatcttaagcgtacgactttgctagtcgctcatattcacaacggccgtcggggcaaagaacgagaggggtacagtgcaggggaaagaataatagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 79 - protein, 288 aa, Artificial
  modified HIV-1 integrase
FLDGIDKAQEEHEKYHSNWRAMASDFNLPPVVAKEIVASCDKCQLKGEAMHGQVDCSPGIWQLGCVHLEGKIILVAVHVASGYIEAEVIPAETGQETAYFILKLAGRWPVKTIHTGNGSNFTSTAVKAACWWAGIKQEFGIPYNPQSQGVVRSMNKELRKIIGQVRDQAEHLKRTTLLVAHIHNGRRGKEREGYSAGERIIDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 80 - DNA, 211 bp, Artificial; identical to SEQ ID NO: 38
  pre-miRNA designed to target the reverse transcriptase of the infectious virus but not of the therapeutic virus

SEQ ID NO: 81 - DNA, 199 bp, Artificial; identical to SEQ ID NO: 50
  pre-miRNA designed to target the reverse transcriptase of the infectious virus but not of the therapeutic virus

SEQ ID NO: 82 - DNA, 177 bp, Artificial
  pre-miRNA designed to target the integrase of the infectious virus but not of the therapeutic virus
agacttcaggaggaagctgctttcactacttttttttttttaattaccatgagggttagaacatttttgtccaatccccccttttcttttaaagagtattcaacatcaagttaatgtctaaccaggtcacatttcgcttcaaaggaacaaaacacatacaaaatgcatgtttttttc

SEQ ID NO: 83 - DNA, 191 bp, Artificial; identical to SEQ ID NO: 75
  pre-miRNA designed to target the integrase of the infectious virus but not of the therapeutic virus

SEQ ID NO: 84 - DNA, 10008 bp, Artificial
  attenuated therapeutic virus against HIV
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagagaaaccagagaagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatagatgggaaaaaattcggctaaggccagggggaaagaaaaaatataaactaaaacatatagtatgggcaagcagggagctagaacgattcgcagttaatcctggcctgttagaaacatcagaaggctgtagacaaatactgggacagctacaaccagcccttcagacaggatcagaagaacttagatcattacataatacagtagcagtcctctattgtgttcatcaaaggatagaggtaaaagacaccaaggaagctttagagaagatagaggaagagcaaaacaaaagtaagaaaaaagcacagcaagcagcagctgacacaggaaacagcagcaaggtcagccaaaattaccctatagtgcagaacatccaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaaggatgtatagtcctaccagcattctggacataagacaaggaccaaaggaaccctttagagactatgtagaccggttctataaaactctaagagccgagcaagcttcacaggaggtaaaaaattggatgacagaaaccttgttggtccaaaattcgaacccagattgtaagactattttaaaagcattgggaccagcagctacactagaagaaatgatgacagcatgtcagggagtgggaggacccagccataaagcaagagttttggctgaagcaatgagccaagcaacaagttcagctaccataatgatgcagagaggcaattttaggaaccaaagaaagattgttaagtgttttaattgtggcaaggaagggcacattgccagaaattgcagggcccctaggaaaaagggctgttggaaatgtggaaaggaaggacaccaaatgaaagactgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggagcagtatcctttagcttccctcagatcactctttggcaacgacccctcgtctcaataaagataggggggcaactaaaggaagctctattagatacaggagcagatgatacagtattagaagaaatgaatttgccaggaagatggaaaccaaaaatgatagggggaattggaggttttatcaaagtaagacagtatgatcagatactcgtagaaatctgtggacataaagctataggtacagtattagtaggacctacacccgtcaacataattggaagaaatctgttgactcagattggttgcactttaaattttcccattagtcctattgaaactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagtctaaagacagtactaaatggcttaaattagtagatttcagagaacttaataagagaactcaagacttctgggaagttcaattaggaataccacatcccgcagggtggaaaaagcagaaatcagagacagtactggaagtgggtgatgcatatttttcagttcccttagataaagacttcaggaagtatactgcatttaccatacctagtataaacaatgagacaccagggattagatatcagtacaatgtgcttgcattttggacaagcaacagtgtgctacaaagtagcatgacaaaaatcttagagccttttagaaaacaaaatccagacatagagatcacccaatacatgacaatattgctcgtaggatctgacttagaaatagggcagcatagaacaaaaatagaggaactgagacaacatctgttgaggtggggatttaccacaccagacaaaaaacatcagaaagaacctccattcctttggatgggttatgaaaagaggagcacgtcctggtctttccagcctatagtgctgccagaaaaagacagctggactgtcaatgacatacagaagttagtgggaaaattgaattgggcaagtcagatttatgcagggattaaagtaaagcaattatgtaaactccttaggggaaccaaagcactaacagaagtagtaccactaacagaagaagcagagctagaactggcagaaaacagggaaattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaatagccacagaaagcatagtaatatggggaaagactcctaaatttaaactacccatacaaaaagaaacatgggaaacatggtggacagagtattggcaagccacctggattcctgagtgggagtttgtcaatacccctcccttagtgaaattatggtaccagttagagaaagaacccatagtaggagcagaaactttctatgtagatggggcagctaatagggagactaaattaggaaaagcaggatatgttactgacagaggaagacaaaaagttgtctccctaactgacacaacaaatcagaagactgagttacaagcaattcatctagctttgcaggattcaggattagaagtaaacatagtaacagactcacaatatgcattaggaatcattcaagcacaaccagataaaagtgaatcagagttagtcagtcaaataatagagcagttaataaaaaaggaaaaggtctacctggcatgggtaccagcacacaaaggaattggaggaaatgaacaagtagataaattagtcagtgctggaatcaggaaagtactatttttagatggaatagataaggcccaagaagaacatgaaaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactaggctgtgtgcatttagaaggaaaaattatcctggtagcagttcatgtagccagtggatatatagaagcagaagttattccagcagaaacagggcaggaaacagcatactttatcttaaaattagcaggaagatggccagtaaaaacaatacatacaggtaatggcagcaatttcaccagtactgcagttaaggccgcctgttggtgggcagggatcaagcaggaatttggcattccctacaatccccaaagtcaaggagtagtacgttctatgaataaagaattacgtaaaattataggacaggtaagagatcaggctgaacatcttaagcgtacgactttgctagtcgctcatattcacaacggccgtcggggcaaagaacgagaggggtacagtgcaggggaaagaataatagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaagaaatggttttatagacatcactatgaaaacactcatcctagaataagttcagaagtgcacatcccactaggggatgctaaattggtaatagcaacatattggggtctgcatacaggagaaagagactggcatttgggccagggagtctccatagaatggaggaaaaagagatatagcacacaagtagaccctggcctagcagaccaactaattcatctgtattattttgattgtttttcagaatctgctataagaaataccatattaggacatagagttagtcctagttgtgaatatcaagcaggacataacaaggtaggatctccacaatacttggcactagctgcattaatagcaccaaaaaagataaagccacctttgcctagtgttacgaaactgacagaggatagatggaacaagccccagaagaccaagggccacagagggagccatacaatgaatggacactagagcttttagaggagcttaagagtgaagctgttaggcattttcctaggttatggctccatagcttaggacaacatatctatgaaacttatggggatacttgggcaggagtggaagccataataagaattctgcaacaactgctgtttattcatttcagaattgggtgtcgacatagcagaataggcataattcgacagaggagagcaagaaatggagccagtagatcctagactagagccctggaagcatccaggaagtcagcctaggactgcttgcaccaattgctattgtaaaaagtgttgccttcattgccaagtttgtttcacaagaaaaggcttaggcatctcctatggcaggaagaagcggagacagcgacgaagagctcctcaagacagtcagactcatcaagcttctctatcaaagcagtaagtagtacatgtaatgcaatctttagaaatattagcaccagtagcattagtagtagcaacaataatagtaatagttgtgtggaccatagtattcatagaatataggaaactattaagacaaagaaaaatagacaggttaattgatagaataagagaaagagcagaagacagtggcaatgaaagcgaaggagaccaggaagaattatcagcacttgtggagatggggcatcacgctccttgggatgttgatgatctgtagtgctgcagaacaattgtgggtcacagtctattatggggtacctgtgtggaaagaagcaaccaccaccctattttgtgcatcagatgctaaagcatatgatacagaggtgcataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaagttaaccccactctgtgttactttaaattgcactgatgatttaaaaaatgctactgtaaaaaatgctactaataccaataatagtagctggggagggatggagagaggagaaataaaaaactgctctttcaatatcaccactagcataagagataaggtgcagaaagaatatgcacttttttataaacttgatgtagtaccaatagataatgctgataataataatattactactaactataccagctatagattgataagttgtaacacctcagtcattacacaggcctgtccaaaggtatcctttgagccaattcccatacattattgtgccccggctggttttgcgattctaaagtgtaacgataagaagttcaatggaacaggaccatgtacaaatgtcagcacagtacaatgtacacatggaattaggccagtagtatcaactcaactgctgttaaatggcagtctagcagaaaaagaggtagtaattagatctgaaaatttcacgaacaatgccaaaaccataatagtacagctggatgaatctgtagtaattaattgtacaagacccaacaacaatacaagaaaaagtatacatataggaccagggagagcattttatacaacaggagaaataataggagatataagacaagctcattgtacccttaatagaacagaatggaataacactttagcaaagataactgaaaaattaagagaacaatttgggaataacataacaatagtctttaatcactcctcgggaggggacccagaaattgtaatgcacagttttatttgtggaggggaatttttctactgtaatacatcacaactgtttaatagtacttggaatagtactgggaataatattagcgagtcagataacactgaaagaaatatcacactcccatgcagaataaaacaaattataaacctgtggcaggaagtaggaaaagcaatgtatgcccctcccatcagaggacaaattagatgttcatcaaatattacagggctgctattaacaagagatggtggtagtaacacggacgagaacaggaccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaacaataggagctatgttccttgggttcttgggagcagcaggaagcactatgggcgcagcgtcgatgacgctgacggtacaggccagactattattgtctggtatagtgcaacagcagaacaatttgctgagggctattgaggcgcaacagcatctgttgcaactcacagtctggggcatcaagcagctccaggcaagagtcctggctgtggaaagatacctaaaggatcaacagctcctagggatttggggttgctctggaaaactcatttgcaccactgctgtgccttggaatgctagttggagtaataaatctctggatgagatttgggataacatgacctggatggaatgggaaagagaaattgacaattacacaaacctaatatacaccttaattgaagaataccggaaccagcaagaaaagaatgaacaagcattattggaattagataaatgggcaagtttgtggaattggtttaacataacaaattggctgtggtatataaaaatattcataatgatagtaggaggcttggtaggtttaagaatagtctttactgtactttctatagtgaatagagttaggaagggatactcaccattatcgtttcagacccgcctcccaaccccgaggggacccgacaggcccgaaggaatcgaagaagaaggtggagagcgagacagagacagatccggtctcttagtggatggattcttagcacttatctgggtcgacctgcggagcctgtgcctcttcagctaccaccgattgagagacttactcttgattgtagcgaggattgtggaacttctgggacgcagggggtgggaagtcctcaaatattggtggaatctcctgcagtattggagtcaggaactaaagaatagtgcagttagcttgctcaatgccacagctatagcagtagctgaggggacagatagggttatagaagtattacaaagagcttgtagagctatcctccacatacctacaagaataagacagggcttggaaagggctttgctataagatgggtggcaagtggtcaaaacgtagtggggttggatggcctactgtaagggaaagaatgagacgagctgagccagcagcagatggggtgggagcagtatctgcctcactggaaaaacatggagcaatcacaagtagcaatacagcagctactaatgctgattgtgcctggctagaagcacaagaggaggaagaggtgggttttccagtcagacctcaggtacctttaagaccaatgacttacaagggagcgttagatcttagccactttttaagagaaaaggggggactggaagggctaatttactcccagaaaagacaagatatccttgatctgtgggtctaccacacacaaggcttcttccctgattggcagaactacacaccagggccagggatcagatacccactgtgttttggatggtgctttaagctagtaccagttgagccagagaaggtagaagaggccaatgaaggagagaacaacagcttgttacaccctatgagcctgcatgggatggagagaccggagaaagaagtgttagtgtggaggtttgacagccgcctagcagttcatcacatggcccgagagcagcatccggagtattacaagaactgctgacagcatgttgtctcctgaatgtatacatcacacccttcttccctgatgctcccattctgcttgctctggtggagatattgctggtgatcctttccaggatctgggttttgaggtcttgggaggatcaccagtgatattttcacatgagtgcatgcacacgggtatggctcttgcttgctgggattacagcttttcagtgccagtgggtgggatccatgagtttttgctaggtttcctttggcaacaattaagaagcaccagtgtgtagattctccttcttcctgtccatttatcaggatggagaagacttttcctttcctccatcctgataaatggacagaggagcatattaaaatattcaacagagggtaattagctgtctatgttttatatgcagtaataatgcttgaagacttcaggaggaagctgctttcactacttttttttttttaattaccatgagggttagaacatttttgtccaatccccccttttcttttaaagagtattcaacatcaaagaaaaggggggatgggaagttaatgtctaaccaggtcacatttcgcttcaaaggaacaaaacacatacaaaatgcatgtttttttcataaaattattaggacaggcaaagcaattcagaggaaagagcatcgcttgggggtaaaatgtttaaagaatactgccatttgtactgctgtgataacaactcagcagtacaaatggcagtatttaaggaatcaatccttgctgcttctgtttatgaagccgagcttcagttttcttgctctgacaatggaccatcgagctttctacaagggactttccgctggggactttccacggaggtgtggccggggcgggactggggagtggcgaaccctcagatgctgcatataagcagctgctttttgcctgtactgggtctctctggttagaccagatctgagcctgggagctctctggctaactagggaacccactgcttaagcctcaataaagcttgccttgagtgcttca

SEQ ID NO: 85 - DNA, 23 bp, Hepatitis C virus
tactctccaggtgaaatcaatag

SEQ ID NO: 86 - protein, 7 aa, Hepatitis C virus
YSPGEIN

SEQ ID NO: 87 - DNA, 23 bp, Artificial
  antisense against ns5b
ctattgatttcacctggagagta

SEQ ID NO: 88 - protein, 5 aa, Artificial
  modified ns5b
VAETN

SEQ ID NO: 89 - DNA, 12 bp, Artificial
  modified ns5b
gtagccgagaca

SEQ ID NO: 90 - DNA, 68 bp, Homo sapiens
taaaaggtagattctccttctatgagtacattatttatgattaatcatagaggaaaatccacgttttc

SEQ ID NO: 91 - DNA, 70 bp, Homo sapiens; identical to SEQ ID NO: 36

SEQ ID NO: 92 - DNA, 210 bp, Pan paniscus; identical to SEQ ID NO: 37

SEQ ID NO: 93 - DNA, 207 bp, Artificial
  pre-miRNA against RDRP of HCV
cagcatgttgtctcctgaatgtatacatcacacccttcttccctgatgctcccattctgcttgctctggtggagatattgatttcacctggagagtacattatttatgattaatctccaggtgaaattgatattttcacatgagtgcatgcacacgggtatggctcttgcttgctgggattacagcttttcagtgccagtgggtggg

SEQ ID NO: 94 - DNA, 8023 bp, Artificial
  therapeutic HCV virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtacccttggcccctctatggcaatgagggctgcgggtgggcgggatggctcctgtccccccgtggctctcggcctagctggggccccacagacccccggcgcaggtcacgcaatttgggtaaggtcatcgataccctcacgtgtggcttcgccgacctcatggggtacattccgctcgtcggcgcccctcttgggggcgctgccagggccctggcgcatggcgtccgggttctggaagacggcgtgaactatgcaacagggaaccttcctggttgctctttctctatcttccttctagccctgctctcttgcctgactgtgcccgcatcagcctaccaagtacgcaactcctcgggcctttaccatgtcaccaatgattgccctaactcgagtattgtgtacgagacggccgacaccattctacactctccggggtgtgttccttgcgttcgcgaggataacgcctcgagatgttgggtgccggtggcccccacagtggccaccagggacggcaaactccccacaacgcagcttcgacgtcacatcgatctgcttgtcgggagcgccaccctctgctcggccctctatgtaggggacttgtgcgggtccgtctttcttgtcagccaactgttcactttttcccccaggcgccactggacaacgcaagactgcaactgttctatctaccccggccatataacgggtcaccgcatggcgtgggatatgatgatgaactggtcccctacggtcgcgctggtaatggctcagctgctcagggtcccacaagccatcttggacatgatcgctggtgctcactggggagtcctagcgggcatagcgtatttctccatggtggggaactgggcgaaggttctggtggtgctgttgctgtttgccggcgttgacgcgcaaacctacgtcaccggggggagtgccgctcggggcgcgtccggactcgccaacctttttacaccgggcgccaagcaggatatccagctgatcaacaccaatggcagttggcacatcaaccgtacggccttgaactgtaatgcgagccttgacactggttgggtcgcagggctcttttattaccataaattcaactcttcaggttgccctgagaggatggccagctgcaaaccccttgccgatttcgaccagggctggggccctattcgccacgccaacggaagcggccccgaacatcgaccctactgctggcactaccccccaaaaccttgtggtatcgtgtcagcacagactgtatgtggcccagtgtattgcttcacccccagccccgtagtggtgggaacgaccaaccggttgggcgtgcccacctacagttggggcaccaatgatacggacgttttcgtccttaataacaccaggccaccgttgggcaattggttcggttgcacttggatgaactcatctggatttaccaaagtgtgcggagcgcctccttgtgtcatcggaggagtgggcaacaacaccctgcactgccccactgactgtttccgcaagcatccggaagccacgtactctcggtgtggctccggtccctggatcacgcccaggtgcctggtccactatccttataggctttggcattatccttgcaccgtcaactacaccctgttcaaagtcaggatgtacgtgggaggggtcgagcacaggctggaagttgcttgcaactggacgcggggcgagcgttgtgatctggacgacagggacaggtccgagctcagcccgctgctgctgtccaccacacagtggcaggtcctcccgtgttctttcacgaccttgccagccttgactaccggcctcatccacctccaccagaacatcgtggacgtgcaatacctgtacggggtggggtcaagcattgtgtcctgggccatcaagtgggaatacgtcatccttctgttcctcctgcttgcagacgcgcgcatctgctcctgcttgtggatgatgctactcatatcccaagcggaggcggctttggagaaccttgtgttactcaatgcagcgtctctggccgggacgcacggtcttgcgtccttcctcgtgttcttctgctttgcatggtatctgaagggtaggtgggtgcccggagtggcctacgccttctacgggatgtggcctctcctcctgctcctgttggcgttgccccagcgggcatacgcgctggacacggagatggccgcgtcgtgtggcggcgttgttcttgtcgggttgatggcgctaactctgtcaccgtactacaagagctatatcagctggtgcttatggtggcttcagtacttcctaactaggatagaagcgctactgcacgtgtgggtcccccccctcaatgtccgaggagggcgcgacgctgtcatcctgctcacgtgtgttgtacacccgaccttggtattcgacatcaccaagctactactggccgtcttcggacccctttggattctccaaaccagtctgctcaaggtgccctacttcgtgcgcgtccaaggccttctccgggcctgcgcgctagcgcgtaaggtggccggaggccattacgtgcagatggtcatcatcaagttgggggcgcttactggcacctacatctacaaccatctcactcctctccgggactgggcgcacaacggcctacgagatctggccgtggctgtagagccagtcgtcttttcccagatggagaccaagctcatcacgtggggggcggacaccgccgcgtgcggtgacatcatcaacggcctgcctgtctctgcccgtaggggccaagagatattgctcggaccggccgatggaatggtctctaaggggtggaggttgctggcgcccatcacggcgtatgcccagcagacaaggggcctcctgggatgcataattactagcctgaccggccgggacaaaaaccaggtggagggtgaggtccagattgtgtcaactgctgcccagaccttcctggcaacctgcatcaacggagtgtgctggactgtctaccacggggccggaacaaggaccatcgcgtcacccaaaggtcccgtcatccagatgtatactaatgtagaccaagaccttgtaggctggcccgctccccaaggtgcccgctcattgacaccctgcacttgcggctcctcggacctttacttggtcacgaggcacgccgatgtcattcccgtgcgccggcggggtgatagcaggggcagcctgctctcgccccggcctatctcttacttgaaaggctcttcggggggcccattgctgtgccccgcgggacacgccgtaggcatattcagggccgcggtgtgcacccgtggagtggctaaggcggtggactttatccccgtagagagtctagagacaaccatgaggtccccggtgttcacagacaactcctccccaccagcagtgccccagagcttccaggtggcccacctgcatgctcccaccggcagcggtaagagtaccaaggtcccggccgcatacgcggctcagggctacaaggtgctggtgctcaacccctccgttgctgcaacactgggctttggtgcttatatgtccaaggcccatgggattgatcctaacatcaggactggggtgaggacaatcactactggcagccccatcacgtactccacctacggcaagttccttgctgatggcgggtgctcggggggtgcttatgacataataatttgtgacgagtgccactctacggatgcaacatccgtcttgggcatcggcactgtccttgaccaagcagagactgcgggggcgagactggtcgtgctcgccaccgctacccctccgggctctgtcactgtgccccatcctaacatcgaggaggttgctctgtccaccaccggagagatccccttttacggcaaggctatcccccttgaggcaatcaagggggggagacatctcatcttctgccattcaaaaaagaagtgcgacgagctcgctgcaaagctggtcgcgatgggcgtcaatgccgtggcttactaccgcggcctcgacgtgtccgtcatcccaaccagtggcgatgttgtcgtcgtggcaactgatgccctcatgaccggctataccggcgactttgactcggtgatagactgcaacacgtgtgtcacccagacagtcgacttcagccttgaccctaccttcaccattgagacaaccacgctcccccaggacgctgtctcccgcactcaacgtcggggcaggactggcagggggaagccaggcatctacagatttgtggcaccgggggagcgcccctccggcatgtttgactcgtccgtcctctgtgagtgctatgacgcgggctgtgcttggtatgagctcacacccgccgagaccacagttaggctacgagcatatatgaacaccccggggctccccgtgtgccaagaccatcttgaattttgggagggcgtcttcacgggtctcacccatatagacgcccatttcctatcccagacaaagcagagtggggaaaaccttccttacctggtagcgtaccaagccaccgtgtgcgctagggctcaggccccccccccatcgtgggaccagatgtggaagtgcttgatccgcctcaagcccacccttcatgggccaacacctctgctatacagactgggcgctgttcagaatgaagtcaccctgacgcacccaatcaccaagtacatcatgacatgcatgtcggctgacctggaggtcgtcacgagtacctgggtgctcgtcggcggcgtcctggctgctttggccgcgtattgcctatccacaggctgcgtggtcatagtaggcaggattgtcttgtccgggaagccggctatcatacctgacagggaagtcctctaccgggagttcgatgagatggaagagtgctctcagcacttgccgtacatcgagcaggggatgatgctcgccgagcagttcaagcagaaggccctcggcctcctgcagaccgcgtcccgccaggcagaggtcatcgcccctaccgtccaaaccaactggcagagactcgaggccttctgggcgaagcatatgtggaacttcatcagtgggatacaatatctggcgggcctgtcaacgttgcctggtaatcccgccattgcatcattgatggcttttacagctgccgtcaccagcccactaaccaccggccaaactctcctcttcaacattttgggggggtgggtggctgcccagctcgcagcccccggtgccgctaccgcctttgtgggcgctggcttagccggcgccgccatcggcagtgttggactggggaaggtcctcgtggacatccttgcagggtatggcgcgggcgtggcgggagctcttgtagcatttaagatcatgagcggtgaggttccctccacagaggacctggtcaatctactgcctgccatcctttcgcccggagcccttgtagtcggtgtggtctgcgcagcaatactacgccggcacgttggcccgggcgagggagcagtgcagtggatgaaccggttgatagccttcgcctcccgggggaaccacgtttcccccacgcactacgtgccggagagcgatgcagctgcccgcgtcactgccatactcagcagcctcactgtgacccagctcctgaggcgactacaccagtggctaagctcggagtgtaccactccatgctccggttcctggctaagggacatctgggactggatatgcgaggtgctgagcgattttaagacctggctgaaggccaagctcatgccacaactgcctgggattccctttgtgtcctgccaacgcgggtacaggggggtctggcgaggagatggcattatgcacactcgctgcccctgtggagctgagatcgccggacatgtcaagaacgggacgatgaggatcgtcggtcctaagacctgcagaaacacgtggagtgggaccttccccatcaacgcctacaccacgggcccctgtaccccccttcctgcgccgaactatacgttcgcgctgtggagggtgtctgcggaggaatacgtggaaataaggcaggtgggggacttccactacgtgacgggcatgactgctgacaaccttaagtgcccatgccaggtcccatcgcccgaatttttcacagaactggatggggtgcgcctgcataggtttgcgcccccttgcaagcccttgctacgagatgaggtgtcgttcagagtaggactacacgactacccggtggggtcgcagttaccttgcgagcctgaaccggatgtggccgtactgacgtccatgctcaccgatccctcccatataacggcagaggcggctgggaggaggttagcaaggggatcgcccccttctctggccagctcctcggccagccagctgtccgctccatctctcaaagcaacttgcaccaccaaccacgactcccctgacgccgagctcatagaggctaacctcctgtggaggcaggagatgggcggcaacatcaccagggttgagtcagagaacaaagtggtagtcctggactccttcgatccgcttgtggcagaagaggacgaacgggagatctccgtggccgcagagatcctgcggaagtctcggagattcgctccggccctgcccatttgggcacggccggactacaaccccccgttactggagacgtggaaaaagccggactacgagccacctgtggtccatggctgcccgcttccacctccaaagtcccctcctgtgcctccgccccggaagaagcggacggtggtcctcactgaatcaactgtatccactgccttggctgagcttgctaccaagagctttggcagctcttcaacttccggtataacgggcgacaacacgacagcgtcctctgagcccgccccctctgtctgccctccagactccgacgctgagtcctattcttccatgccccccctggagggggagcctggggatccggatctcagcgacgggtcatggtcgacggtcagtagtggggctggcacggaggatgtcgtgtgttgctcataacagcatgttgtctcctgaatgtatacatcacacccttcttccctgatgctcccattctgcttgctctggtggagatattgatttcacctggagagtacattatttatgattaatctccaggtgaaattgatattttcacatgagtgcatgcacacgggtatggctcttgcttgctgggattacagcttttcagtgccagtgggtgggacactccggcctcttaggccattcctgttttttttttttttttcttttgttttttttgtttttttttttttttttcctttctttttttttttttttcctttcttctttaatggtggctccatcttagccctagtcacggctagctgtgaaaggtccgtgagccgcatgactgcagagagtgctgatactggcctctctgcagatcatgt

SEQ ID NO: 95 - DNA, 9802 bp, Artificial
  therapeutic HCV virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtacccttggcccctctatggcaatgagggctgcgggtgggcgggatggctcctgtccccccgtggctctcggcctagctggggccccacagacccccggcgcaggtcacgcaatttgggtaaggtcatcgataccctcacgtgtggcttcgccgacctcatggggtacattccgctcgtcggcgcccctcttgggggcgctgccagggccctggcgcatggcgtccgggttctggaagacggcgtgaactatgcaacagggaaccttcctggttgctctttctctatcttccttctagccctgctctcttgcctgactgtgcccgcatcagcctaccaagtacgcaactcctcgggcctttaccatgtcaccaatgattgccctaactcgagtattgtgtacgagacggccgacaccattctacactctccggggtgtgttccttgcgttcgcgaggataacgcctcgagatgttgggtgccggtggcccccacagtggccaccagggacggcaaactccccacaacgcagcttcgacgtcacatcgatctgcttgtcgggagcgccaccctctgctcggccctctatgtaggggacttgtgcgggtccgtctttcttgtcagccaactgttcactttttcccccaggcgccactggacaacgcaagactgcaactgttctatctaccccggccatataacgggtcaccgcatggcgtgggatatgatgatgaactggtcccctacggtcgcgctggtaatggctcagctgctcagggtcccacaagccatcttggacatgatcgctggtgctcactggggagtcctagcgggcatagcgtatttctccatggtggggaactgggcgaaggttctggtggtgctgttgctgtttgccggcgttgacgcgcaaacctacgtcaccggggggagtgccgctcggggcgcgtccggactcgccaacctttttacaccgggcgccaagcaggatatccagctgatcaacaccaatggcagttggcacatcaaccgtacggccttgaactgtaatgcgagccttgacactggttgggtcgcagggctcttttattaccataaattcaactcttcaggttgccctgagaggatggccagctgcaaaccccttgccgatttcgaccagggctggggccctattcgccacgccaacggaagcggccccgaacatcgaccctactgctggcactaccccccaaaaccttgtggtatcgtgtcagcacagactgtatgtggcccagtgtattgcttcacccccagccccgtagtggtgggaacgaccaaccggttgggcgtgcccacctacagttggggcaccaatgatacggacgttttcgtccttaataacaccaggccaccgttgggcaattggttcggttgcacttggatgaactcatctggatttaccaaagtgtgcggagcgcctccttgtgtcatcggaggagtgggcaacaacaccctgcactgccccactgactgtttccgcaagcatccggaagccacgtactctcggtgtggctccggtccctggatcacgcccaggtgcctggtccactatccttataggctttggcattatccttgcaccgtcaactacaccctgttcaaagtcaggatgtacgtgggaggggtcgagcacaggctggaagttgcttgcaactggacgcggggcgagcgttgtgatctggacgacagggacaggtccgagctcagcccgctgctgctgtccaccacacagtggcaggtcctcccgtgttctttcacgaccttgccagccttgactaccggcctcatccacctccaccagaacatcgtggacgtgcaatacctgtacggggtggggtcaagcattgtgtcctgggccatcaagtgggaatacgtcatccttctgttcctcctgcttgcagacgcgcgcatctgctcctgcttgtggatgatgctactcatatcccaagcggaggcggctttggagaaccttgtgttactcaatgcagcgtctctggccgggacgcacggtcttgcgtccttcctcgtgttcttctgctttgcatggtatctgaagggtaggtgggtgcccggagtggcctacgccttctacgggatgtggcctctcctcctgctcctgttggcgttgccccagcgggcatacgcgctggacacggagatggccgcgtcgtgtggcggcgttgttcttgtcgggttgatggcgctaactctgtcaccgtactacaagagctatatcagctggtgcttatggtggcttcagtacttcctaactaggatagaagcgctactgcacgtgtgggtcccccccctcaatgtccgaggagggcgcgacgctgtcatcctgctcacgtgtgttgtacacccgaccttggtattcgacatcaccaagctactactggccgtcttcggacccctttggattctccaaaccagtctgctcaaggtgccctacttcgtgcgcgtccaaggccttctccgggcctgcgcgctagcgcgtaaggtggccggaggccattacgtgcagatggtcatcatcaagttgggggcgcttactggcacctacatctacaaccatctcactcctctccgggactgggcgcacaacggcctacgagatctggccgtggctgtagagccagtcgtcttttcccagatggagaccaagctcatcacgtggggggcggacaccgccgcgtgcggtgacatcatcaacggcctgcctgtctctgcccgtaggggccaagagatattgctcggaccggccgatggaatggtctctaaggggtggaggttgctggcgcccatcacggcgtatgcccagcagacaaggggcctcctgggatgcataattactagcctgaccggccgggacaaaaaccaggtggagggtgaggtccagattgtgtcaactgctgcccagaccttcctggcaacctgcatcaacggagtgtgctggactgtctaccacggggccggaacaaggaccatcgcgtcacccaaaggtcccgtcatccagatgtatactaatgtagaccaagaccttgtaggctggcccgctccccaaggtgcccgctcattgacaccctgcacttgcggctcctcggacctttacttggtcacgaggcacgccgatgtcattcccgtgcgccggcggggtgatagcaggggcagcctgctctcgccccggcctatctcttacttgaaaggctcttcggggggcccattgctgtgccccgcgggacacgccgtaggcatattcagggccgcggtgtgcacccgtggagtggctaaggcggtggactttatccccgtagagagtctagagacaaccatgaggtccccggtgttcacagacaactcctccccaccagcagtgccccagagcttccaggtggcccacctgcatgctcccaccggcagcggtaagagtaccaaggtcccggccgcatacgcggctcagggctacaaggtgctggtgctcaacccctccgttgctgcaacactgggctttggtgcttatatgtccaaggcccatgggattgatcctaacatcaggactggggtgaggacaatcactactggcagccccatcacgtactccacctacggcaagttccttgctgatggcgggtgctcggggggtgcttatgacataataatttgtgacgagtgccactctacggatgcaacatccgtcttgggcatcggcactgtccttgaccaagcagagactgcgggggcgagactggtcgtgctcgccaccgctacccctccgggctctgtcactgtgccccatcctaacatcgaggaggttgctctgtccaccaccggagagatccccttttacggcaaggctatcccccttgaggcaatcaagggggggagacatctcatcttctgccattcaaaaaagaagtgcgacgagctcgctgcaaagctggtcgcgatgggcgtcaatgccgtggcttactaccgcggcctcgacgtgtccgtcatcccaaccagtggcgatgttgtcgtcgtggcaactgatgccctcatgaccggctataccggcgactttgactcggtgatagactgcaacacgtgtgtcacccagacagtcgacttcagccttgaccctaccttcaccattgagacaaccacgctcccccaggacgctgtctcccgcactcaacgtcggggcaggactggcagggggaagccaggcatctacagatttgtggcaccgggggagcgcccctccggcatgtttgactcgtccgtcctctgtgagtgctatgacgcgggctgtgcttggtatgagctcacacccgccgagaccacagttaggctacgagcatatatgaacaccccggggctccccgtgtgccaagaccatcttgaattttgggagggcgtcttcacgggtctcacccatatagacgcccatttcctatcccagacaaagcagagtggggaaaaccttccttacctggtagcgtaccaagccaccgtgtgcgctagggctcaggccccccccccatcgtgggaccagatgtggaagtgcttgatccgcctcaagcccacccttcatgggccaacacctctgctatacagactgggcgctgttcagaatgaagtcaccctgacgcacccaatcaccaagtacatcatgacatgcatgtcggctgacctggaggtcgtcacgagtacctgggtgctcgtcggcggcgtcctggctgctttggccgcgtattgcctatccacaggctgcgtggtcatagtaggcaggattgtcttgtccgggaagccggctatcatacctgacagggaagtcctctaccgggagttcgatgagatggaagagtgctctcagcacttgccgtacatcgagcaggggatgatgctcgccgagcagttcaagcagaaggccctcggcctcctgcagaccgcgtcccgccaggcagaggtcatcgcccctaccgtccaaaccaactggcagagactcgaggccttctgggcgaagcatatgtggaacttcatcagtgggatacaatatctggcgggcctgtcaacgttgcctggtaatcccgccattgcatcattgatggcttttacagctgccgtcaccagcccactaaccaccggccaaactctcctcttcaacattttgggggggtgggtggctgcccagctcgcagcccccggtgccgctaccgcctttgtgggcgctggcttagccggcgccgccatcggcagtgttggactggggaaggtcctcgtggacatccttgcagggtatggcgcgggcgtggcgggagctcttgtagcatttaagatcatgagcggtgaggttccctccacagaggacctggtcaatctactgcctgccatcctttcgcccggagcccttgtagtcggtgtggtctgcgcagcaatactacgccggcacgttggcccgggcgagggagcagtgcagtggatgaaccggttgatagccttcgcctcccgggggaaccacgtttcccccacgcactacgtgccggagagcgatgcagctgcccgcgtcactgccatactcagcagcctcactgtgacccagctcctgaggcgactacaccagtggctaagctcggagtgtaccactccatgctccggttcctggctaagggacatctgggactggatatgcgaggtgctgagcgattttaagacctggctgaaggccaagctcatgccacaactgcctgggattccctttgtgtcctgccaacgcgggtacaggggggtctggcgaggagatggcattatgcacactcgctgcccctgtggagctgagatcgccggacatgtcaagaacgggacgatgaggatcgtcggtcctaagacctgcagaaacacgtggagtgggaccttccccatcaacgcctacaccacgggcccctgtaccccccttcctgcgccgaactatacgttcgcgctgtggagggtgtctgcggaggaatacgtggaaataaggcaggtgggggacttccactacgtgacgggcatgactgctgacaaccttaagtgcccatgccaggtcccatcgcccgaatttttcacagaactggatggggtgcgcctgcataggtttgcgcccccttgcaagcccttgctacgagatgaggtgtcgttcagagtaggactacacgactacccggtggggtcgcagttaccttgcgagcctgaaccggatgtggccgtactgacgtccatgctcaccgatccctcccatataacggcagaggcggctgggaggaggttagcaaggggatcgcccccttctctggccagctcctcggccagccagctgtccgctccatctctcaaagcaacttgcaccaccaaccacgactcccctgacgccgagctcatagaggctaacctcctgtggaggcaggagatgggcggcaacatcaccagggttgagtcagagaacaaagtggtagtcctggactccttcgatccgcttgtggcagaagaggacgaacgggagatctccgtggccgcagagatcctgcggaagtctcggagattcgctccggccctgcccatttgggcacggccggactacaaccccccgttactggagacgtggaaaaagccggactacgagccacctgtggtccatggctgcccgcttccacctccaaagtcccctcctgtgcctccgccccggaagaagcggacggtggtcctcactgaatcaactgtatccactgccttggctgagcttgctaccaagagctttggcagctcttcaacttccggtataacgggcgacaacacgacagcgtcctctgagcccgccccctctgtctgccctccagactccgacgctgagtcctattcttccatgccccccctggagggggagcctggggatccggatctcagcgacgggtcatggtcgacggtcagtagtggggctggcacggaggatgtcgtgtgttgctcaatgtcctattcctggacaggcgcactcatcaccccgtgtgccgcggaagaacaaaaattgcctatcaacgcactgagcaactcattactgcgtcaccacaacctcgtgtattccaccacctcacgcagtgcttgccaaaggcagaagaaagtcacatttgacagactgcaagttctggacaaccactaccaggacgtgctcaaggaggttaaggcggcggcgtcaaaagtgaaggctaacttgctatccgtagaggaagcttgcagcctgacgcccccacattcagccagatcaaaatttggctatggggcaaaagacgtccgttgccatgccagaaaggccgtaaaccacatcaactccgtgtggaaagaccttctggaagacagtgttacaccaatagacacaaccatcatggctaagaacgaagttttctgcgttcagcctgagaaggggggtcgtaagccagctcgtctcatcgtgtaccctgacctgggtgtgcgcgtgtgcgagaaaatggccctgtacgacgtggtaaaaaaactccccctggccgtgatgggaagctcctacggattccagtactcaccaggacagcgggttgaattcctcgtgcaagcgtggaagtccaaggggaccccaatggggttctcgtatgatacccgctgctttgactctacagtcactgagagcgatatccgtacggaggaggcaatctaccagtgttgtgacctggacccccaagcccgcgtggccatcaagtccctcaccgagaggctttatgtcgggggtcctcttaccaattcaaggggggaaaactgcggctatcgcaggtgccgcgcaagcggcgtactgacaactagctgtggtaacaccctcacttgctacatcaaggcccgagcagcctgtcgagccgcagggctccgggactgcaccatgctcgtgtgtggcgacgacttagtcgttatctgtgaaagccagggggtccaagaggatacagcgagcctgagagccttcacggaggctatgaccaggtactccgctccccccggggaccccccccaaccagaatacgacttggagctcataacatcgtgctcctctaacgtgtcagtcgcccacgacgacactggaaagagggtctattaccttacccgtgaccctacaactcccctcgcgagagccgcgtgggagacagcaagacacactccagtcaattcctggctaggcaacataatcatgtttgcccccacattgtgggtgagaatgatactgctgccccacttcttcagtgtcctcatggccagggaccaacttgaacaggcccttgattgcgaaatctacggagcctgctactccatagaaccactggacctacctccaatcattcaaagactccatggccttagcgcattttcactccacagtgtagccgagacaaatagggtggccgcatgcctcagaaaacttggggtcccgcccttgcgagcttggagacaccgggcccggagcatccgcgctaagcttctgtccagaggaggcagggctgccacgtgtggcaagtacctcttcaattgggcagtaagaacaaagctcaaactcactccaatagcggccgctagccagctggacttgtccggttggttcacggctggctacagcgggggagacatttatcacagcgtgtctcgtgcccggccccgctggttctggttttgcctactcctgcttgctgcaggggtaggcatctacctcctccccaaccgatgacagcatgttgtctcctgaatgtatacatcacacccttcttccctgatgctcccattctgcttgctctggtggagatattgatttcacctggagagtacattatttatgattaatctccaggtgaaattgatattttcacatgagtgcatgcacacgggtatggctcttgcttgctgggattacagcttttcagtgccagtgggtgggtgaaggttggggtaaacactccggcctcttaggccattcctgttttttttttttttttcttttgttttttttgtttttttttttttttttcctttctttttttttttttttcctttcttctttaatggtggctccatcttagccctagtcacggctagctgtgaaaggtccgtgagccgcatgactgcagagagtgctgatactggcctctctgcagatcatgt

SEQ ID NO: 96 - RNA, 74 nt, Homo sapiens
acuccuucugcagcucugguggaaaauggagaagacuuuuccuuuccuccaucucccccagggccugguggagu

SEQ ID NO: 97 - RNA, 49 nt, Homo sapiens
cuguaauauaaauuuaauuuauucucuaucauuaaaaaauguauuacag